(19) United States
(12) Patent Application Publication
(10) Pub. No.: US 2023/0321087 A1
(43) Pub. Date: Oct. 12, 2023

(54) FGFR INHIBITOR COMBINATION THERAPIES

(71) Applicant: JANSSEN PHARMACEUTICA NV, Beerse (BE)

(72) Inventors: Ademi Elena SANTIAGO-WALKER, Spring House, PA (US); Christopher H. MOY, Raritan, NJ (US)

(21) Appl. No.: 18/044,232

(22) PCT Filed: Sep. 13, 2021
(86) PCT No.: PCT/EP2021/075145

§ 371 Date: Mar. 7, 2023

Related U.S. Application Data
(60) Provisional application No. 63/078,193, filed on Sep. 14, 2020.

(51) Int. Cl. A61K 31/498; A61K 39/395; A61P 35/00

(52) U.S. Cl. CPC A61K 31/498; A61K 39/3955; A61P 35/00

(57) ABSTRACT

Described herein are methods of treating cancer comprising administering a fibroblast growth factor receptor (FGFR) inhibitor in combination with an epidermal growth factor receptor (EGFR) inhibitor, a Cyclin D1 (CCND1) inhibitor or a BRAF inhibitor to a patient in need of cancer treatment, wherein the patient harbors at least one FGFR2 genetic alteration or FGFR3 genetic alteration and at least one EGFR, CCND1 or BRAF genetic alteration, respectively. Also described herein are methods of predicting duration of progression-free survival (PFS) or overall survival (OS) in a patient, in particular a human patient, having cancer, in particular in a patient on treatment with an FGFR inhibitor, the method comprising evaluating a biological sample from the patient for the presence of at least one FGFR2 genetic alteration or FGFR3 genetic alteration and at least one EGFR, CCND1, or BRAF genetic alteration, wherein the presence of at least one FGFR2 genetic alteration or FGFR3 genetic alteration and at least one EGFR, CCND1, or BRAF genetic alteration indicates a shorter duration of PFS or a shorter duration of OS, relative to a patient, in particular a human patient, having cancer who does not harbor at least one EGFR, CCND1, or BRAF genetic alteration, respectively, or relative to a patient, in particular a human patient, having cancer who does not harbor at least one FGFR2 genetic alteration or FGFR3 genetic alteration and at least one EGFR, CCND1, or BRAF genetic alteration, respectively. Additionally, described herein are methods of improving PFS or OS in a patient with cancer relative to a patient with cancer who was not receiving treatment with an FGFR inhibitor in combination with an EGFR inhibitor, a CCND1 inhibitor or a BRAF inhibitor, said method comprising providing to said patient an FGFR inhibitor in combination with an EGFR inhibitor, a CCND1 inhibitor or a BRAF inhibitor, wherein said patient harbors at least one FGFR2 genetic alteration or FGFR3 genetic alteration and at least one EGFR, CCND1, or BRAF genetic alteration, respectively.

### TECHNICAL FIELD

Disclosed herein are methods of treating cancer comprising administering a fibroblast growth factor receptor (FGFR) inhibitor in combination with an epidermal growth factor receptor (EGFR) inhibitor, a Cyclin D1 (CCND1) inhibitor or a BRAF inhibitor to a patient in need of cancer treatment, wherein the patient harbors at least one FGFR2 genetic alteration or FGFR3 genetic alteration and at least one EGFR, CCND1 or BRAF genetic alteration, respectively. Also disclosed herein are methods of predicting duration of progression-free survival (PFS) or overall survival (OS) in a patient, in particular a human patient, having cancer, in particular in a patient on treatment with an FGFR inhibitor, the method comprising evaluating a biological sample from the patient for the presence of at least one FGFR2 genetic alteration or FGFR3 genetic alteration and at least one EGFR, CCND1, or BRAF genetic alteration, wherein the presence of at least one FGFR2 genetic alteration or FGFR3 genetic alteration and at least one EGFR, CCND1, or BRAF genetic alteration indicates a shorter duration of PFS or a shorter duration of OS, relative to a patient, in particular a human patient, having cancer who does not harbor at least one EGFR, CCND1, or BRAF genetic alteration, respectively, or relative to a patient, in particular a human patient, having cancer who does not harbor at least one FGFR2 genetic alteration or FGFR3 genetic alteration and at least one EGFR, CCND1, or BRAF genetic alteration, respectively. Additionally, disclosed herein are methods of improving PFS or OS in a patient with cancer relative to a patient with cancer who was not receiving treatment with an FGFR inhibitor in combination with an EGFR inhibitor, a CCND1 inhibitor or a BRAF inhibitor, said method comprising providing to said patient an FGFR inhibitor in combination with an EGFR inhibitor, a CCND1 inhibitor or a BRAF inhibitor, wherein said patient harbors at least one FGFR2 genetic alteration or FGFR3 genetic alteration and at least one EGFR, CCND1, or BRAF genetic alteration, respectively.

### BACKGROUND

Treatment with the pan-FGFR inhibitor erdafitinib provides benefits for patients with locally advanced or metastatic urothelial carcinoma (mUC). Loriot Y, et al. N Engl J Med. 2019; 381:338-348. Circulating tumor DNA (ctDNA) analysis is a noninvasive method to identify somatic gene alterations present in tumors. Morales-Barrera R, et al. Transl Androl Urol. 2018; 7:S101-S103; Lodewijk I, et al. Int J Mol Sci. 2018; 19:2514. Results from a phase 2, multicenter, open-label study (NCT02365597) of erdafitinib in patients with locally advanced or metastatic UC and FGFR2/3 alterations led to approval of erdafitinib by the US Food and Drug Administration as the first targeted therapy approved for mUC. Loriot Y, et al. N Engl J Med. 2019; 381:338-348; Marandino L, et al. Expert Rev Anticancer Ther. 2019; 19:835-846. To identify markers of intrinsic resistance to erdafitinib, ctDNA from plasma samples from patients in BLC2001 was analyzed using next-generation sequencing (NGS). There is a need for combination therapies that are efficacious in patients with FGFR2/3 alterations plus a marker of intrinsic resistance to erdafitinib.

### SUMMARY

Described herein are methods of treating cancer comprising administering an FGFR inhibitor in combination with an EGFR inhibitor to a patient in need of cancer treatment, wherein the patient harbors at least one FGFR2 genetic alteration or FGFR3 genetic alteration and at least one EGFR genetic alteration.

Also described herein are methods of treating cancer in a patient comprising: (a) evaluating a biological sample from the patient for the presence of at least one FGFR2 genetic alteration or FGFR3 genetic alteration and at least one EGFR genetic alteration; and (b) treating the patient with an FGFR inhibitor in combination with an EGFR inhibitor if the at least one FGFR2 genetic alteration or FGFR3 genetic alteration and the at least one EGFR genetic alteration are present in the sample.

Further described herein are methods of predicting duration of PFS in a patient, in particular a human patient, having cancer, in particular in a patient on treatment with an FGFR inhibitor, the method comprising evaluating a biological sample from the patient for the presence of at least one FGFR2 genetic alteration or FGFR3 genetic alteration and at least one EGFR genetic alteration, wherein the presence of at least one FGFR2 genetic alteration or FGFR3 genetic alteration and at least one EGFR genetic alteration indicates a shorter duration of PFS, relative to a patient, in particular a human patient, having cancer who does not harbor at least one EGFR genetic alteration, or relative to a patient, in particular a human patient, having cancer who does not harbor at least one FGFR2 genetic alteration or FGFR3 genetic alteration and at least one EGFR genetic alteration.

Still further described herein are methods of predicting duration of OS in a patient, in particular a human patient, having cancer, in particular in a patient on treatment with an FGFR inhibitor, the method comprising evaluating a biological sample from the patient for the presence of at least one FGFR2 genetic alteration or FGFR3 genetic alteration and at least one EGFR genetic alteration, wherein the presence of at least one FGFR2 genetic alteration or FGFR3 genetic alteration and at least one EGFR genetic alteration indicates a shorter duration of OS relative to a patient, in particular a human patient, having cancer who does not harbor at least one EGFR genetic alteration, or relative to a patient, in particular a human patient, having cancer who does not harbor at least one FGFR2 genetic alteration or FGFR3 genetic alteration and at least one EGFR genetic alteration.

Also described herein are methods of improving OS in a patient with cancer relative to a patient with cancer who was not receiving treatment with an FGFR inhibitor in combination with an EGFR inhibitor, said method comprising providing to said patient an FGFR inhibitor in combination with an EGFR inhibitor, wherein said patient harbors at least one FGFR2 genetic alteration or FGFR3 genetic alteration and at least one EGFR genetic alteration.

Further described herein are methods of improving PFS in a patient with cancer relative to a patient with cancer who was not receiving treatment with an FGFR inhibitor in combination with an EGFR inhibitor, said method comprising providing to said patient an FGFR inhibitor in combination with an EGFR inhibitor, wherein said patient harbors at least one FGFR2 genetic alteration or FGFR3 genetic alteration and at least one EGFR genetic alteration.

Still further described herein are an FGFR inhibitor and an EGFR inhibitor for use in the treatment of cancer in a patient who harbors at least one FGFR2 genetic alteration or FGFR3 genetic alteration and at least one EGFR genetic alteration. Also described herein is an FGFR inhibitor for use in the treatment of cancer in a patient who harbors at least one FGFR2 genetic alteration or FGFR3 genetic alteration and at least one EGFR genetic alteration, wherein the FGFR inhibitor is to be used in combination with an EGFR inhibitor. Also described herein is an EGFR inhibitor for use in the treatment of cancer in a patient who harbors at least one FGFR2 genetic alteration or FGFR3 genetic alteration and at least one EGFR genetic alteration, wherein the EGFR inhibitor is to be used in combination with an FGFR inhibitor

Described herein are uses of an FGFR inhibitor for the manufacture of a medicament for the treatment of cancer in a patient who harbors at least one FGFR2 genetic alteration or FGFR3 genetic alteration and at least one EGFR genetic alteration, wherein the FGFR inhibitor is to be used in combination with an EGFR inhibitor.

Also described herein are uses of an EGFR inhibitors for the manufacture of a medicament for the treatment of cancer, in particular urothelial carcinoma, in a patient who harbors at least one FGFR2 genetic alteration or FGFR3 genetic alteration and at least one EGFR genetic alteration, wherein the EGFR inhibitor is to be used in combination with an FGFR inhibitor.

Further described herein are methods of treating cancer comprising administering an FGFR inhibitor in combination with a CCND1 inhibitor to a patient in need of cancer treatment, wherein the patient harbors at least one FGFR2 genetic alteration or FGFR3 genetic alteration and at least one CCND1 genetic alteration.

Still further described herein are methods of treating cancer in a patient comprising: (a) evaluating a biological sample from the patient for the presence of at least one FGFR2 genetic alteration or FGFR3 genetic alteration and at least one CCND1 genetic alteration; and (b) treating the patient with an FGFR inhibitor in combination with a CCND1 inhibitor if the at least one FGFR2 genetic alteration or FGFR3 genetic alteration and the at least one CCND1 genetic alteration are present in the sample.

Described herein are methods of predicting duration of PFS in a patient, in particular a human patient, having cancer, in particular in a patient on treatment with a FGFR inhibitor, the method comprising evaluating a biological sample from the patient for the presence of at least one FGFR2 genetic alteration or FGFR3 genetic alteration and at least one CCND1 genetic alteration, wherein the presence of at least one FGFR2 genetic alteration or FGFR3 genetic alteration and at least one CCND1 genetic alteration indicates a shorter duration of PFS relative to a patient, in particular a human patient, having cancer who does not harbor at least one CCND1 genetic alteration, or relative to a patient, in particular a human patient, having cancer who does not harbor at least one FGFR2 genetic alteration or FGFR3 genetic alteration and at least one CCND1 genetic alteration.

Also described herein are methods of improving PFS in a patient with cancer relative to a patient with cancer who was not receiving treatment with an FGFR inhibitor in combination with a CCND1 inhibitor, said method comprising providing to said patient an FGFR inhibitor in combination with a CCND1 inhibitor, wherein said patient harbors at least one FGFR2 genetic alteration or FGFR3 genetic alteration and at least one CCND1 genetic alteration.

Further described herein are an FGFR inhibitor and a CCND1 inhibitor for use in the treatment of cancer in a patient who harbors at least one FGFR2 genetic alteration or FGFR3 genetic alteration and at least one CCND1 genetic alteration. Also described herein is an FGFR inhibitor for use in the treatment of cancer in a patient who harbors at least one FGFR2 genetic alteration or FGFR3 genetic alteration and at least one CCND1 genetic alteration, wherein the FGFR inhibitor is to be used in combination with a CCND1 inhibitor. Also described herein is a CCND1 inhibitor for use in the treatment of cancer in a patient who harbors at least one FGFR2 genetic alteration or FGFR3 genetic alteration and at least one CCND1 genetic alteration, wherein the CCND1 inhibitor is to be used in combination with an FGFR inhibitor.

Still further described herein are uses of an FGFR inhibitor for the manufacture of a medicament for the treatment of cancer in a patient who harbors at least one FGFR2 genetic alteration or FGFR3 genetic alteration and at least one CCND1 genetic alteration, wherein the FGFR inhibitor is to be used in combination with a CCND1 inhibitor.

Described herein are uses of a CCND1 inhibitor for the manufacture of a medicament for the treatment of cancer, in particular urothelial carcinoma, in a patient who harbors at least one FGFR2 genetic alteration or FGFR3 genetic alteration and at least one CCND1 genetic alteration, wherein the CCND1 inhibitor is to be used in combination with an FGFR inhibitor.

Further described herein are methods of treating cancer comprising administering an FGFR inhibitor in combination with a BRAF inhibitor to a patient in need of cancer treatment, wherein the patient harbors at least one FGFR2 genetic alteration or FGFR3 genetic alteration and at least one BRAF genetic alteration.

Still further described herein are methods of treating cancer in a patient comprising: (a) evaluating a biological sample from the patient for the presence of at least one FGFR2 genetic alteration or FGFR3 genetic alteration and at least one BRAF genetic alteration; and (b) treating the patient with an FGFR inhibitor in combination with a BRAF inhibitor if the at least one FGFR2 genetic alteration or FGFR3 genetic alteration and the at least one BRAF genetic alteration are present in the sample.

Described herein are methods of predicting duration of PFS in a patient, in particular a human patient, having cancer, in particular in a patient on treatment with an FGFR inhibitor, the method comprising evaluating a biological sample from the patient for the presence of at least one FGFR2 genetic alteration or FGFR3 genetic alteration and at least one BRAF genetic alteration, wherein the presence of at least one FGFR2 genetic alteration or FGFR3 genetic alteration and at least one BRAF genetic alteration indicates a shorter duration of PFS relative to a patient, in particular a human patient, having cancer who does not harbor at least one BRAF genetic alteration, or relative to a patient, in particular a human patient, having cancer who does not harbor at least one FGFR2 genetic alteration or FGFR3 genetic alteration and at least one BRAF genetic alteration.

Also described herein are methods of improving PFS in a patient with cancer relative to a patient with cancer who was not receiving treatment with an FGFR inhibitor in combination with a BRAF inhibitor, said method comprising providing to said patient an FGFR inhibitor in combination with a BRAF inhibitor, wherein said patient harbors at least one FGFR2 genetic alteration or FGFR3 genetic alteration and at least one BRAF genetic alteration.

Further described herein are an FGFR inhibitor and a BRAF inhibitor for use in the treatment of cancer in a patient who harbors at least one FGFR2 genetic alteration or FGFR3 genetic alteration and at least one BRAF genetic alteration. Also described herein is an FGFR inhibitor for use in the treatment of cancer in a patient who harbors at least one FGFR2 genetic alteration or FGFR3 genetic alteration and at least one BRAF genetic alteration, wherein the FGFR inhibitor is to be used in combination with a BRAF inhibitor. Also described herein is a BRAF inhibitor for use in the treatment of cancer in a patient who harbors at least one FGFR2 genetic alteration or FGFR3 genetic alteration and at least one BRAF genetic alteration, wherein the BRAF inhibitor is to be used in combination with an FGFR inhibitor

Still further described herein are uses of an FGFR inhibitor for the manufacture of a medicament for the treatment of cancer in a patient who harbors at least one FGFR2 genetic alteration or FGFR3 genetic alteration and at least one BRAF genetic alteration, wherein the FGFR inhibitor is to be used in combination with a BRAF inhibitor.

Described herein are uses of a BRAF inhibitor for the manufacture of a medicament for the treatment of cancer, in particular urothelial carcinoma, in a patient who harbors at least one FGFR2 genetic alteration or FGFR3 genetic alteration and at least one BRAF genetic alteration, wherein the BRAF inhibitor is to be used in combination with an FGFR inhibitor.

### BRIEF DESCRIPTION OF THE DRAWINGS

The summary, as well as the following detailed description, is further understood when read in conjunction with the appended drawings. For the purpose of illustrating the disclosed methods and uses, the drawings show exemplary embodiments of the methods and uses; however, the methods and uses are not limited to the specific embodiments disclosed. In the drawings:

FIG. 1 is a schematic of the phase 2, multicenter, open-label study (NCT02365597) described in Example 1. A=dose uptitration if ≥5.5 mg/dL target serum phosphate not reached by Day 14 and if no treatment-related adverse events.

FIG. 2A is a Kaplan-Meier curve that depicts progress free survival by EGFR alteration status with survival probability or strata (EGFR alteration present or not present) on the y-axis and PFS in months on the x-axis.

FIG. 2B is a Kaplan-Meier curve that depicts progress free survival by CCND1 alteration status with survival probability or strata (CCND1 alteration present or not present) on the y-axis and PFS in months on the x-axis.

FIG. 2C is a Kaplan-Meier curve that depicts progress free survival by BRAF alteration status with survival probability or strata (BRAF alteration present or not present) on the y-axis and PFS in months on the x-axis.

FIG. 3 is a Kaplan-Meier curve that depicts OS by EGFR alteration status with survival probability or strata (EGFR alteration present or not present) on the y-axis and OS in months on the x-axis

FIG. 4 shows the gene alteration profile in ctDNA at baseline.

FIG. 5 depicts co-expression data for genes associated with shorter PFS. Intersection size (y-axis) is provided for BRAF, EGFR, CCND1 and wild-type (WT) subjects.

### DETAILED DESCRIPTION OF ILLUSTRATIVE EMBODIMENTS

It is to be appreciated that certain features of the invention which are, for clarity, described herein in the context of separate embodiments may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, although an embodiment may be described as part of a series of steps or part of a more general structure, each said step may also be considered an independent embodiment in itself, combinable with others.

### Certain Terminology

The transitional terms “comprising”, “consisting essentially of”, and “consisting” are intended to connote their generally in accepted meanings in the patent vernacular; that is, (i) “comprising”, which is synonymous with “including”, “containing”, or “characterized by”, is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps and those that do not materially affect the basic and novel characteristic(s) of the claimed invention. More specifically, the basic and novel characteristics relates to the ability of the method to provide at least one of the benefits described herein, including but not limited to the ability to improve the survivability of the human population relative to the survivability of the comparative human population described elsewhere herein. Embodiments described in terms of the phrase “comprising” (or its equivalents), also provide, as embodiments, those which are independently described in terms of “consisting of” and “consisting essentially of”.

When a value is expressed as an approximation by use of the descriptor “about”, it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about”. In other cases, the gradations used in a series of values may be used to determine the intended range available to the term “about” for each value. Where present, all ranges are inclusive and combinable. That is, references to values stated in ranges include every value within that range.

If not otherwise specified, the term “about” signifies a variance of ±10% of the associated value, but additional embodiments include those where the variance may be ±5%, ±15%, ±20%, ±25%, or ±50%.

When a list is presented, unless stated otherwise, it is to be understood that each individual element of that list, and every combination of that list, is a separate embodiment. For example, a list of embodiments presented as “A, B, or C” is to be interpreted as including the embodiments, “A”, “B”, “C”, “A or B”, “A or C”, “B or C”, or “A, B, or C”.

As used herein, the singular forms “a”, “an”, and “the” include the plural.

As used herein, the term “at least one” means “one or more.”

As used herein, “patient” is intended to mean any animal, in particular, mammals. Thus, the methods are applicable to human and nonhuman animals, although most preferably with humans. The terms “patient” and “subject” and “human” may be used interchangeably.

The terms “treat” and “treatment” refer to the treatment of a patient afflicted with a pathological condition and refers to an effect that alleviates the condition by killing the cancerous cells, but also to an effect that results in the inhibition of the progress of the condition, and includes a reduction in the rate of progress, a halt in the rate of progress, amelioration of the condition, and cure of the condition. Treatment as a prophylactic measure (i.e., prophylaxis) is also included.

The term “dose” refers to the amount or quantity of the therapeutic to be taken each time.

The term “cancer” as used herein refers to an abnormal growth of cells which tend to proliferate in an uncontrolled way and, in some cases, to metastasize (spread).

The terms “co-administration”, “administered in combination” or the like, as used herein, encompass administration of the selected therapeutic agents to a single patient, and are intended to include treatment regimens in which the agents are administered by the same or different route of administration or at the same or different time. The selected therapeutic agents can be administered simultaneously, concurrently or sequentially. As used herein “sequentially” refers to administering the EGFR, BRAF, CCND1, ARID1A, ErbB2 or TERT inhibitor to a patient which is or was on treatment with an FGFR inhibitor. In an embodiment, the selected therapeutic agents are administered simultaneously or concurrent. In an embodiment, the selected therapeutic agents are administered early in the treatment, in particular in the treatment of locally advanced or metastatic urothelial carcinoma.

The term “continuous daily dosing schedule” refers to the administration of a particular therapeutic agent without any drug holidays from the particular therapeutic agent. In some embodiments, a continuous daily dosing schedule of a particular therapeutic agent comprises administration of a particular therapeutic agent every day at roughly the same time each day.

The term “progression-free survival” (PFS) is defined as the time from first dose to date of documented evidence of disease progression or death, whichever comes first.

The term “overall survival” (OS) is defined as the time from the date of randomization to the date of the participant's death resulting from any cause.

The term “placebo” as used herein means administration of a pharmaceutical composition that does not include an FGFR inhibitor.

The term “randomization” as it refers to a clinical trial refers to the time when the patient is confirmed eligible for the clinical trial and gets assigned to a treatment arm.

The terms “kit” and “article of manufacture” are used as synonyms.

“Biological samples” refers to any sample from a patient in which cancerous cells can be obtained and detection of a specified genetic alteration is possible. Suitable biological samples include, but are not limited to, blood, lymph fluid, bone marrow, a solid tumor sample, or any combination thereof. In some embodiments, the biological sample can be formalin-fixed paraffin-embedded tissue (FFPET), in particular a formalin-fixed paraffin-embedded tumor tissue.

The term “adverse event” is any untoward medical event that occurs in a participant administered an investigational product, and it does not necessarily indicate only events with clear causal relationship with the relevant investigational product.

As used herein, the term “cell-free DNA” (cfDNA) refers to short segments of DNA that are shed into the bloodstream during cell turnover.

As used herein, the term “circulating tumor DNA” (ctDNA) refers to short segments of DNA that are shed into the bloodstream during cell turnover that may be derived from primary tumors, metastatic lesions, or circulating tumor cells (CTCs).

### Genetic Alterations

FGFR Genetic Alterations

The fibroblast growth factor (FGF) family of protein tyrosine kinase (PTK) receptors regulates a diverse array of physiologic functions including mitogenesis, wound healing, cell differentiation and angiogenesis, and development. Both normal and malignant cell growth as well as proliferation are affected by changes in local concentration of FGFs, extracellular signaling molecules which act as autocrine as well as paracrine factors. Autocrine FGF signaling may be particularly important in the progression of steroid hormone-dependent cancers to a hormone independent state.

The following abbreviations are used throughout the disclosure: FGFR (fibroblast growth factor receptor); FGFR3-TACC3 v1 (fusion between genes encoding FGFR3 and transforming acidic coiled-coil containing protein 3 variant 1, also referred to herein as FGFR3-TACC3 V1); FGFR3-TACC3 v3 (fusion between genes encoding FGFR3 and transforming acidic coiled-coil containing protein 3 variant 3, also referred to herein as FGFR3-TACC3_V3); FGFR3-BAIAP2L1 (fusion between genes encoding FGFR3 and brain-specific angiogenesis inhibitor 1-associated protein 2-like protein 1); FGFR2-BICC1 (fusion between genes encoding FGFR2 and bicaudal C homolog 1); FGFR2-CASP7 (fusion between genes encoding FGFR2 and caspase 7).

FGFs and their receptors are expressed at increased levels in several tissues and cell lines and overexpression is believed to contribute to the malignant phenotype. Furthermore, a number of oncogenes are homologues of genes encoding growth factor receptors, and there is a potential for aberrant activation of FGF-dependent signaling in human pancreatic cancer (Knights et al., Pharmacology and Therapeutics 2010 125:1 (105-117); Korc M. et al Current Cancer Drug Targets 2009 9:5 (639-651)).

The two prototypic members are acidic fibroblast growth factor (aFGF or FGF1) and basic fibroblast growth factor (bFGF or FGF2), and to date, at least twenty distinct FGF family members have been identified. The cellular response to FGFs is transmitted via four types of high affinity transmembrane protein tyrosine-kinase FGFR numbered 1 to 4 (FGFR1 to FGFR4).

As used herein, “FGFR genetic alteration” refers to an alteration in the wild type FGFR gene, including, but not limited to, FGFR fusion genes, FGFR mutations, FGFR amplifications, or any combination thereof. In certain embodiments, the FGFR amplifications are copy number amplifications. The terms “variant” and “alteration” are used interchangeably herein.

In certain embodiments, the FGFR2 or FGFR3 genetic alteration is an FGFR gene fusion. “FGFR fusion” or “FGFR gene fusion” refers to a gene encoding a portion of FGFR (e.g., FGFR2 or FGFR3) and one of the herein disclosed fusion partners, or a portion thereof, created by a translocation between the two genes. The terms “fusion” and “translocation” are used interchangeable herein. The presence of one or more of the following FGFR fusion genes in a biological sample from a patient can be determined using the disclosed methods or uses: FGFR3-TACC3, FGFR3-BAIAP2L1, FGFR2-BICC1, FGFR2-CASP7, or any combination thereof. In certain embodiments, FGFR3-TACC3 is FGFR3-TACC3 variant 1 (FGFR3-TACC3 V1) or FGFR3-TACC3 variant 3 (FGFR3-TACC3 V3). Table 1 provides the FGFR fusion genes and the FGFR and fusion partner exons that are fused. The sequences of the individual FGFR fusion genes are disclosed in Table 4.

TABLE 1

| Fusion Gene | FGFR Exon | Partner Exon |
|---|---|---|
| FGFR2 | | |
| FGFR2-BICC1 | 19 | 3 |
| FGFR2-CASP7 | 19 | 4 |
| FGFR3 | | |
| FGFR3-BAIAP2L1 | 18 | 2 |
| FGFR3-TACC3 V1 | 18 | 11 |
| FGFR3-TACC3 V3 | 18 | 10 |

FGFR genetic alterations include FGFR single nucleotide polymorphism (SNP). “FGFR single nucleotide polymorphism” (SNP) refers to an FGFR2 or FGFR3 gene in which a single nucleotide differs among individuals. In certain embodiments, the FGFR2 or FGFR3 genetic alteration is an FGFR3 gene mutation. In particular, FGFR single nucleotide polymorphism” (SNP) refers to an FGFR3 gene in which a single nucleotide differs among individuals. The presence of one or more of the following FGFR SNPs in a biological sample from a patient can be determined by methods known to those of ordinary skill in the art or methods disclosed in WO 2016/048833, FGFR3 R248C, FGFR3 S249C, FGFR3 G370C, FGFR3 Y373C, or any combination thereof. The sequences of the FGFR SNPs are provided in Table 2.

TABLE 2

| FGFR3 mutant | Sequence |
|---|---|
| FGFR3 R248C | TCGGACCGCGGCAACTACACCTGCGTCGTGGAGAACAAG |
| | TTTGGCAGCATCCGGCAGACGTACACGCTGGACGTGCTGG |
| | AG(T)GCTCCCCGCACCGGCCCATCCTGCAGGCGGGGCTGC |
| | CGGCCAACCAGACGGCGGTGCTGGGCAGCGACGTGGAGT |
| | TCCACTGCAAGGTGTACAGTGACGCACAGCCCCACATCCA |
| | GTGGCTCAAGCACGTGGAGGTGAATGGCAGCAAGGTGGG |
| | CCCGGACGGCACACCCTACGTTACCGTGCTCA |
| | (SEQ ID NO: 1) |
| FGFR3 S249C | GACCGCGGCAACTACACCTGCGTCGTGGAGAACAAGTTTG |
| | GCAGCATCCGGCAGACGTACACGCTGGACGTGCTGGGTG |
| | AGGGCCCTGGGGCGGCGCGGGGGTGGGGGCGGCAGTGGC |
| | GGTGGTGGTGAGGGAGGGGGTGGCCCCTGAGCGTCATCT |
| | GCCCCCACAGAGCGCT(G)CCCGCACCGGCCCATCCTGCAG |
| | GCGGGGCTGCCGGCCAACCAGACGGCGGTGCTGGGCAGC |
| | GACGTGGAGTTCCACTGCAAGGTGTACAGTGACGCACAG |
| | CCCCACATCCAGTGGCTCAAGCACGTGGAGGTGAATGGC |
| | AGCAAGGTGGGCCCGGACGGCACACCCTACGTTACCGTG |
| | CTCAAGGTGGGCCACCGTGTGCACGT |
| | (SEQ ID NO: 2) |
| FGFR3 G370C | GCGGGCAATTCTATTGGGTTTTCTCATCACTCTGCGTGGCT |
| | GGTGGTGCTGCCAGCCGAGGAGGAGCTGGTGGAGGCTGA |
| | CGAGGCG(T)GCAGTGTGTATGCAGGCATCCTCAGCTACGG |
| | GGTGGGCTTCTTCCTGTTCATCCTGGTGGTGGCGGCTGTG |
| | ACGCTCTGCCGCCTGCGCAGCCCCCCCAAGAAAGGCCTGG |
| | GCTCCCCCACCGTGCACAAGATCTCCCGCTTCCCG |
| | (SEQ ID NO: 3) |
| FGFR3 Y373C* | CTAGAGGTTCTCTCCTTGCACAACGTCACCTTTGAGGACG |
| | CCGGGGAGTACACCTGCCTGGCGGGCAATTCTATTGGGTT |
| | TTCTCATCACTCTGCGTGGCTGGTGGTGCTGCCAGCCGAG |
| | GAGGAGCTGGTGGAGGCTGACGAGGCGGGCAGTGTGT(G) |
| | TGCAGGCATCCTCAGCTACGGGGTGGGCTTCTTCCTGTTC |
| | ATCCTGGTGGTGGCGGCTGTGACGCTCTGCCGCCTGCGCA |
| | GCCCCCCCAAGAAAGGCCTGGGCTCCCCCACCGTGCACAA |
| | GATCTCCCGCTTCCCGCTCAAGC |
| | (SEQ ID NO: 4) |

Sequences correspond to nucleotides 920-1510 of FGFR3 (Genebank ID# NM_000142.4).
Nucleotides in bold underline represent the SNP.
*Sometimes mistakenly referred to as Y375C in the literature.

As used herein, “FGFR genetic alteration gene panel” includes one or more of the above listed FGFR genetic alterations. In some embodiments, the FGFR genetic alteration gene panel is dependent upon the patient's cancer type.

The FGFR genetic alteration gene panel that is used in the evaluating step of the disclosed methods is based, in part, on the patient's cancer type. For patients with urothelial carcinoma, in particular locally advanced or metastatic UC, a suitable FGFR genetic alteration gene panel can comprise FGFR3-TACC3 V1, FGFR3-TACC3 V3, FGFR3-BAIAP2L1, FGFR2-BICC1, FGFR2-CASP7, FGFR3 R248C, FGFR3 S249C, FGFR3 G370C, or FGFR3 Y373C, or any combination thereof.

In certain embodiments, the patient harbors at least one FGFR2 genetic alteration or FGFR3 genetic alteration and at least one FGFR1 genetic alteration. In certain embodiments, the patient harbors at least one FGFR2 genetic alteration or FGFR3 genetic alteration, at least one FGFR1 genetic alteration, and at least one EGFR genetic alteration. In certain embodiments, the patient harbors at least one FGFR2 genetic alteration or FGFR3 genetic alteration, at least one FGFR1 genetic alteration, and at least one BRAF genetic alteration. In certain embodiments, the patient harbors at least one FGFR2 genetic alteration or FGFR3 genetic alteration, at least one FGFR1 genetic alteration, and at least one CCND1 genetic alteration. In certain embodiments, the patient harbors at least one FGFR2 genetic alteration or FGFR3 genetic alteration, at least one FGFR1 genetic alteration, and at least one ARID1A genetic alteration. In certain embodiments, the patient harbors at least one FGFR2 genetic alteration or FGFR3 genetic alteration, at least one FGFR1 genetic alteration, and at least one TERT genetic alteration. In certain embodiments, the patient harbors at least one FGFR2 genetic alteration or FGFR3 genetic alteration, at least one FGFR1 genetic alteration, and at least one ErbB2 genetic alteration. In certain embodiments, the patient harbors at least one FGFR2 genetic alteration or FGFR3 genetic alteration, at least one FGFR1 genetic alteration, at least one EGFR genetic alteration, at least one CCND1 genetic alteration, at least one ARID1A genetic alteration, at least one TERT genetic alteration, at least one ErbB2 genetic alteration, or any combination thereof.

In certain embodiments, the patient harbors at least one FGFR2 genetic alteration or FGFR3 genetic alteration, at least one FGFR1 genetic alteration, at least one BRAF genetic alteration, at least one EGFR genetic alteration, at least one CCND1 genetic alteration, at least one ARID1A genetic alteration, at least one TERT genetic alteration, at least one ErbB2 genetic alteration, or any combination thereof.

EGFR Alterations

Described herein are methods of treating cancer comprising administering an FGFR inhibitor in combination with an EGFR inhibitor to a patient in need of cancer treatment, wherein the patient harbors at least one FGFR2 genetic alteration or FGFR3 genetic alteration and at least one EGFR genetic alteration. In certain embodiments the cancer is mUC.

“Epidermal growth factor receptor” or “EGFR” as used here refers to the human EGFR (also known as HER1 or ErbB1 (Ullrich et al., Nature 309:418-425, 1984) having the amino acid sequence shown in SEQ ID NO: 38 and in GenBank accession number NP_005219, as well as naturally-occurring variants thereof. Such variants include well-known EGFRvIII and other alternatively spliced variants (e.g., as identified by SwissProt Accession numbers P00533-1 (wild type; identical to SEQ ID NO: 38 and NP_005219), P00533-2 (F404L/L4055), P00533-3 (628-705: CTGPGLEGCP (SEQ ID NO: 45) . . . GEAPNQALLR (SEQ ID NO: 46)→PGNESLKAML (SEQ ID NO: 47) . . . SVIITASSCH (SEQ ID NO: 48) and 706-1210 deleted), P00533-4 (C628S and 629-1210 deleted), variants GlnQ98, R266, K521, I674, G962, and P988 (Livingston et al., NIEHS-SNPs, environmental genome project, NIEHS ES15478), T790M, L858R/T790M and del(E746, A750).

“EGFR ligand” as used herein encompasses all (e.g., physiological) ligands for EGFR, including EGF, TGFα, heparin binding EGF (HB-EGF), amphiregulin (AR), and epiregulin (EPI).

“Epidermal growth factor” (EGF) as used herein refers to the well-known 53 amino acid human EGF having the amino acid sequence shown in SEQ ID NO: 39.

As used herein, “EGFR genetic alteration” refers to an alteration in the wild type EGFR gene, including, but not limited to, EGFR mutations, EGFR amplifications, EGFR gene insertions, or any combination thereof. In certain embodiments, the EGFR amplifications are copy number amplifications.

In certain embodiments, the EGFR genetic alteration is a gene mutation. EGFR gene mutations include EGFR single nucleotide polymorphism (SNP). “EGFR single nucleotide polymorphism” (SNP) refers to an EGFR gene in which a single nucleotide differs among individuals. In certain embodiments, the EGFR gene mutation is a K80N substitution. In certain embodiments, the EGFR genetic alteration is a gene insertion. In some embodiments, the gene insertion is an N771_H773dup insertion.

In certain embodiments, the patient harbors at least one FGFR2 genetic alteration or FGFR3 genetic alteration and at least one EGFR genetic alteration. In certain embodiments, the patient harbors at least one FGFR2 genetic alteration or FGFR3 genetic alteration, at least one EGFR genetic alteration, and at least one FGFR1 genetic alteration. In certain embodiments, the patient harbors at least one FGFR2 genetic alteration or FGFR3 genetic alteration, at least one EGFR genetic alteration, and at least one BRAF genetic alteration. In certain embodiments, the patient harbors at least one FGFR2 genetic alteration or FGFR3 genetic alteration, at least one EGFR genetic alteration, and at least one CCND1 genetic alteration. In certain embodiments, the patient harbors at least one FGFR2 genetic alteration or FGFR3 genetic alteration, at least one EGFR genetic alteration, and at least one ARID1A genetic alteration. In certain embodiments, the patient harbors at least one FGFR2 genetic alteration or FGFR3 genetic alteration, at least one EGFR genetic alteration, and at least one TERT genetic alteration. In certain embodiments, the patient harbors at least one FGFR2 genetic alteration or FGFR3 genetic alteration, at least one EGFR genetic alteration, and at least one ErbB2 genetic alteration.

BRAF Genetic Alterations

Further described herein are methods of treating cancer comprising administering an FGFR inhibitor in combination with a BRAF inhibitor to a patient in need of cancer treatment, wherein the patient harbors at least one FGFR2 genetic alteration or FGFR3 genetic alteration and at least one BRAF genetic alteration. In certain embodiments the cancer is mUC.

As used herein, the terms “BRAF”, “B-Raf”, “Braf” and “BRaf” may be used interchangeably. BRAF is a signal transduction protein kinase involved in the regulation of the mitogen-activated protein kinase (MAPK or ERK) signaling pathway. In some embodiments, gene BRAF may be identified as GENBANK accession number NM_004333.5, NR_148928.1 or NM_001354609.1.

As used herein, “BRAF genetic alteration” refers to an alteration in the wild type BRAF gene, including, but not limited to, BRAF mutations, BRAF amplifications, BRAF gene insertions, or any combination thereof. In certain embodiments, the BRAF amplifications are copy number amplifications

In certain embodiments, the BRAF genetic alteration is a gene mutation. BRAF gene mutations include BRAF single nucleotide polymorphism (SNP). “BRAF single nucleotide polymorphism” (SNP) refers to a BRAF gene in which a single nucleotide differs among individuals. In certain embodiments, the BRAF gene mutation is a D594G substitution, a K601E substitution, or any combination thereof. In certain embodiments, the BRAF gene mutation is a D594G substitution. In certain embodiments, the BRAF gene mutation is a K601E substitution.

In certain embodiments, the patient harbors at least one FGFR2 genetic alteration or FGFR3 genetic alteration and at least one BRAF genetic alteration. In certain embodiments, the patient harbors at least one FGFR2 genetic alteration or FGFR3 genetic alteration, at least one BRAF genetic alteration, and at least one FGFR1 genetic alteration. In certain embodiments, the patient harbors at least one FGFR2 genetic alteration or FGFR3 genetic alteration, at least one BRAF genetic alteration, and at least one EGFR genetic alteration. In certain embodiments, the patient harbors at least one FGFR2 genetic alteration or FGFR3 genetic alteration, at least one BRAF genetic alteration, and at least one CCND1 genetic alteration. In certain embodiments, the patient harbors at least one FGFR2 genetic alteration or FGFR3 genetic alteration, at least one BRAF genetic alteration, and at least one ARID1A genetic alteration. In certain embodiments, the patient harbors at least one FGFR2 genetic alteration or FGFR3 genetic alteration, at least one BRAF genetic alteration, and at least one TERT genetic alteration. In certain embodiments, the patient harbors at least one FGFR2 genetic alteration or FGFR3 genetic alteration, at least one BRAF genetic alteration, and at least one ErbB2 genetic alteration.

CCND1 Genetic Alterations

Further described herein are methods of treating cancer comprising administering an FGFR inhibitor in combination with a CCND1 inhibitor to a patient in need of cancer treatment, wherein the patient harbors at least one FGFR2 genetic alteration or FGFR3 genetic alteration and at least one CCND1 genetic alteration. In certain embodiments the cancer is mUC.

In certain embodiments, the patient harbors at least one CCND1 genetic alteration. In humans, the CCND1 gene encodes the G1/S-specific cyclin-D1 protein. The G1/S-specific cyclin-D1 belongs to the highly conserved cyclin family, whose members are characterized by periodicity in protein abundance throughout the cell cycle. Cyclins function as regulators of CDKs (cyclin-dependent kinase).

As used herein, “CCND1 genetic alteration” refers to an alteration in the wild type CCND1 gene, including, but not limited to, CCND1 mutations, CCND1 amplifications, CCND1 gene insertions, or any combination thereof. In certain embodiments, the CCND1 amplifications are copy number amplifications

In certain embodiments, the CCND1 genetic alteration is a gene mutation. CCND1 gene mutations include CCND1 single nucleotide polymorphism (SNP). “CCND1 single nucleotide polymorphism” (SNP) refers to a CCND1 gene in which a single nucleotide differs among individuals. In certain embodiments, the CCND1 gene mutation is a CCND1 gene amplification.

In certain embodiments, the patient harbors at least one FGFR2 genetic alteration or FGFR3 genetic alteration and at least one CCND1 genetic alteration. In certain embodiments, the patient harbors at least one FGFR2 genetic alteration or FGFR3 genetic alteration, at least one CCND1 genetic alteration, and at least one FGFR1 genetic alteration. In certain embodiments, the patient harbors at least one FGFR2 genetic alteration or FGFR3 genetic alteration, at least one CCND1 genetic alteration, and at least one EGFR genetic alteration. In certain embodiments, the patient harbors at least one FGFR2 genetic alteration or FGFR3 genetic alteration, at least one CCND1 genetic alteration, and at least one BRAF genetic alteration. In certain embodiments, the patient harbors at least one FGFR2 genetic alteration or FGFR3 genetic alteration, at least one CCND1 genetic alteration, and at least one ARID1A genetic alteration. In certain embodiments, the patient harbors at least one FGFR2 genetic alteration or FGFR3 genetic alteration, at least one CCND1 genetic alteration, and at least one TERT genetic alteration. In certain embodiments, the patient harbors at least one FGFR2 genetic alteration or FGFR3 genetic alteration, at least one CCND1 genetic alteration, and at least one ErbB2 genetic alteration.

ARID1A Genetic Alterations

Further described herein are methods of treating cancer comprising administering an FGFR inhibitor in combination with an AT-rich interactive domain-containing protein 1A (ARID1A) inhibitor to a patient in need of cancer treatment, wherein the patient harbors at least one FGFR2 genetic alteration or FGFR3 genetic alteration and at least one ARID1A genetic alteration. In certain embodiments the cancer is mUC.

ARID1A is a component of the BRG1-associated factor (BAF) chromatin remodeling complex.

“ARID1A genetic alteration” refers to an alteration in the wild type ARID1A gene, including, but not limited to, ARID1A mutations, ARID1A amplifications, ARID1A gene insertions, or any combination thereof. In certain embodiments, the ARID1A amplifications are copy number amplifications

In certain embodiments, the ARID1A genetic alteration is a gene mutation. ARID1A gene mutations include ARID1A single nucleotide polymorphism (SNP). “ARID1A single nucleotide polymorphism” (SNP) refers to an ARID1A gene in which a single nucleotide differs among individuals. In certain embodiments, the ARID1A gene mutation is a Q288* loss of function mutation, a Q524* loss of function mutation, an H1881fs loss of function mutation, a F1750fs loss of function mutation, a Q585E substitution, a Q1401K substitution, a P392P substitution, or any combination thereof. In certain embodiments, the ARID1A gene mutation is a Q288* loss of function mutation. In certain embodiments, the ARID1A gene mutation is a Q524* loss of function mutation. In certain embodiments, the ARID1A gene mutation is a H1881fs loss of function mutation. In certain embodiments, the ARID1A gene mutation is a F1750fs loss of function mutation. In certain embodiments, the ARID1A gene mutation is a Q585E substitution. In certain embodiments, the ARID1A gene mutation is a Q1401K substitution. In certain embodiments, the ARID1A gene mutation is a P392P substitution.

In certain embodiments, the patient harbors at least one FGFR2 genetic alteration or FGFR3 genetic alteration and at least one ARID1A genetic alteration. In certain embodiments, the patient harbors at least one FGFR2 genetic alteration or FGFR3 genetic alteration, at least one ARID1A genetic alteration, and at least one FGFR1 genetic alteration. In certain embodiments, the patient harbors at least one FGFR2 genetic alteration or FGFR3 genetic alteration, at least one ARID1A genetic alteration, and at least one EGFR genetic alteration. In certain embodiments, the patient harbors at least one FGFR2 genetic alteration or FGFR3 genetic alteration, at least one ARID1A genetic alteration, and at least one BRAF genetic alteration. In certain embodiments, the patient harbors at least one FGFR2 genetic alteration or FGFR3 genetic alteration, at least one ARID1A genetic alteration, and at least one CCND1 genetic alteration. In certain embodiments, the patient harbors at least one FGFR2 genetic alteration or FGFR3 genetic alteration, at least one ARID1A genetic alteration, and at least one TERT genetic alteration. In certain embodiments, the patient harbors at least one FGFR2 genetic alteration or FGFR3 genetic alteration, at least one ARID1A genetic alteration, and at least one ErbB2 genetic alteration.

ErbB2 Genetic Alterations

Further described herein are methods of treating cancer comprising administering an FGFR inhibitor in combination with an ErbB2 inhibitor to a patient in need of cancer treatment, wherein the patient harbors at least one FGFR2 genetic alteration or FGFR3 genetic alteration and at least one ErbB2 genetic alteration. In certain embodiments the cancer is mUC.

ErbB receptors belong to the family of receptor tyrosine kinases, and they are composed of an extracellular ligand binding domain, a single transmembrane domain, and an intracellular domain with tyrosine kinase activity. The ErbB family comprises ErbB1 (also known as EGFR), ErbB2 (also known as HER2 or neu), ErbB3 (also known as HER3), and ErbB4 (also known as HER4).

As used herein, “ErbB2 genetic alteration” refers to an alteration in the wild type ErbB2 gene, including, but not limited to, ErbB2 mutations, ErbB2 amplifications, ErbB2 gene insertions, or any combination thereof. In certain embodiments, the ErbB2 amplifications are copy number amplifications

In certain embodiments, the ErbB2 genetic alteration is a gene mutation. ErbB2 gene mutations include ErbB2 single nucleotide polymorphism (SNP). “ErbB2 single nucleotide polymorphism” (SNP) refers to an ErbB2 gene in which a single nucleotide differs among individuals. In certain embodiments, the ErbB2 genetic alteration is an S310F substitution, an S250C substitution, an S423T substitution, or any combination thereof. In certain embodiments, the ErbB2 genetic alteration is an S310F substitution. In certain embodiments, the ErbB2 genetic alteration is an S250C substitution. In certain embodiments, the ErbB2 genetic alteration is an S423T substitution.

In certain embodiments, the patient harbors at least one FGFR2 genetic alteration or FGFR3 genetic alteration and at least one ErbB2 genetic alteration. In certain embodiments, the patient harbors at least one FGFR2 genetic alteration or FGFR3 genetic alteration, at least one ErbB2 genetic alteration, and at least one FGFR1 genetic alteration. In certain embodiments, the patient harbors at least one FGFR2 genetic alteration or FGFR3 genetic alteration, at least one ErbB2 genetic alteration, and at least one EGFR genetic alteration. In certain embodiments, the patient harbors at least one FGFR2 genetic alteration or FGFR3 genetic alteration, at least one ErbB2 genetic alteration, and at least one BRAF genetic alteration. In certain embodiments, the patient harbors at least one FGFR2 genetic alteration or FGFR3 genetic alteration, at least one ErbB2 genetic alteration, and at least one CCND1 genetic alteration. In certain embodiments, the patient harbors at least one FGFR2 genetic alteration or FGFR3 genetic alteration, at least one ErbB2 genetic alteration, and at least one TERT genetic alteration. In certain embodiments, the patient harbors at least one FGFR2 genetic alteration or FGFR3 genetic alteration, at least one ErbB2 genetic alteration, and at least one ARID1A genetic alteration.

TERT Genetic Alterations

Further described herein are methods of treating cancer comprising administering an FGFR inhibitor in combination with a TERT inhibitor to a patient in need of cancer treatment, wherein the patient harbors at least one FGFR2 genetic alteration or FGFR3 genetic alteration and at least one TERT genetic alteration. In certain embodiments the cancer is mUC.

As used herein, the term “TERT” refers to telomerase reverse transcriptase, also known as TP2, TRT, EST2, TCS1 and hEST2. TERT is a catalytic subunit of the enzyme telomerase, which, together with the telomerase RNA component (TERC), comprises the most important unit of the telomerase complex. The human TERT gene is located in chromosome 5 and has accession number NM_001193376.1 in the GenBank database.

As used herein, “TERT genetic alteration” refers to an alteration in the wild type TERT gene, including, but not limited to, TERT mutations, TERT amplifications, TERT gene insertions, or any combination thereof. In certain embodiments, the TERT amplifications are copy number amplifications

In certain embodiments, the TERT genetic alteration is a gene mutation. TERT gene mutations include TERT single nucleotide polymorphism (SNP). “TERT single nucleotide polymorphism” (SNP) refers to an TERT gene in which a single nucleotide differs among individuals. In certain embodiments, the TERT genetic alteration is a Y667N substitution, an intronic promoter mutation, or any combination thereof. In certain embodiments, the TERT genetic alteration is a Y667N substitution. In certain embodiments, the TERT genetic alteration is an intronic promoter mutation.

In certain embodiments, the patient harbors at least one FGFR2 genetic alteration or FGFR3 genetic alteration and at least one TERT genetic alteration. In certain embodiments, the patient harbors at least one FGFR2 genetic alteration or FGFR3 genetic alteration, at least one TERT genetic alteration, and at least one FGFR1 genetic alteration. In certain embodiments, the patient harbors at least one FGFR2 genetic alteration or FGFR3 genetic alteration, at least one TERT genetic alteration, and at least one EGFR genetic alteration. In certain embodiments, the patient harbors at least one FGFR2 genetic alteration or FGFR3 genetic alteration, at least one TERT genetic alteration, and at least one BRAF genetic alteration. In certain embodiments, the patient harbors at least one FGFR2 genetic alteration or FGFR3 genetic alteration, at least one TERT genetic alteration, and at least one CCND1 genetic alteration. In certain embodiments, the patient harbors at least one FGFR2 genetic alteration or FGFR3 genetic alteration, at least one TERT genetic alteration, and at least one ErbB2 genetic alteration. In certain embodiments, the patient harbors at least one FGFR2 genetic alteration or FGFR3 genetic alteration, at least one TERT genetic alteration, and at least one ARID1A genetic alteration.

### Inhibitors for Use in the Disclosed Methods or Uses

FGFR Inhibitors

Suitable FGFR inhibitors for use in the disclosed methods and uses are provided herein. The FGFR inhibitors may be used alone or in combination with one or more additional FGFR inhibitors, EGFR inhibitors, BRAF inhibitors, CCND1 inhibitors, ARID1A inhibitors, ErbB2 inhibitors, or TERT inhibitors, in the treatment methods and uses described herein.

In some embodiments, if one or more FGFR genetic alterations are present in the sample, the urothelial carcinoma can be treated with an FGFR inhibitor disclosed in U.S. Publication No. 2013/0072457 A1 (incorporated herein by reference), including any tautomeric or stereochemically isomeric form thereof, and a N-oxide thereof, a pharmaceutically acceptable salt thereof, or a solvate thereof.

In some aspects, for example, the cancer, in particular the urothelial carcinoma, may be treated with N-(3,5-dimethoxyphenyl)-N′-(1-methylethyl)-N-[3-(1-methyl-1H-pyrazol-4-yl)quinoxalin-6-yl]ethane-1,2-diamine (referred to herein “JNJ-42756493” or “JNJ493” or erdafitinib), including any tautomeric form thereof, N-oxides thereof, pharmaceutically acceptable salts thereof, or solvates thereof. In some embodiments, the FGFR inhibitor can be the compound of formula (I), also referred to as erdafitinib:

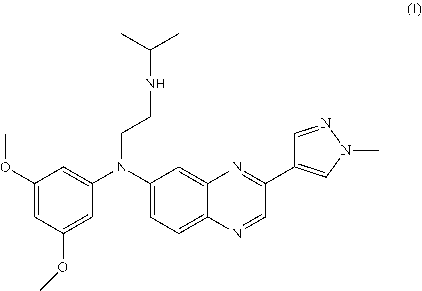

or a pharmaceutically acceptable salt thereof. In some aspects, the pharmaceutically acceptable salt is a HCl salt. In preferred aspects, erdafitinib base is used.

Erdafitinib (also referred to as ERDA), a once-daily oral pan-FGFR kinase inhibitor, has been approved by the U.S. Food and Drug Administration (FDA) for the treatment of adult patients who have locally advanced UC or mUC which has susceptible FGFR3 or FGFR2 genetic alterations and who have progressed during or following at least one line of prior platinum-containing chemotherapy, including within 12 months of neoadjuvant or adjuvant platinum-containing chemotherapy. Loriot Y et al. NEJM. 2019; 381:338-48. Erdafitinib has shown clinical benefits and tolerability in patients with mUC and alteration in FGFR expressions. Tabernero J, et al. J Clin Oncol. 2015; 33:3401-3408; Soria J-C, et al. Ann Oncol. 2016; 27(Suppl 6):vi266-vi295. Abstract 781PD; Siefker-Radtke A O, et al. ASCO 2018. Abstract 4503; Siefker-Radtke A, et al. ASCO-GU 2018. Abstract 450.

In some embodiments, the cancer, in particular the urothelial carcinoma, can be treated with an FGFR inhibitor wherein the FGFR inhibitor is N-[5-[2-(3,5-Dimethoxyphenyl)ethyl]-2H-pyrazol-3-yl]-4-(3,5-diemthylpiperazin-1-yl)benzamide (AZD4547), as described in Gavine, P. R., et al., AZD4547: An Orally Bioavailable, Potent, and Selective Inhibitor of the FGFR Tyrosine Kinase Family, Cancer Res. Apr. 15, 2012 72; 2045:

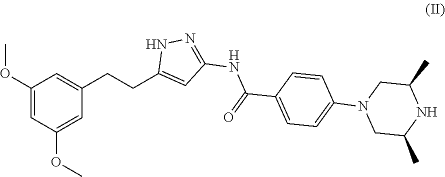 [SMILES: COc1cc(CCc2cc(NC(=O)c3ccc(N4C[C@@H](C)N[C@@H](C)C4)cc3)n[nH]2)cc(OC)c1]

including, when chemically possible, any tautomeric or stereochemically isomeric form thereof, and a N-oxide thereof, a pharmaceutically acceptable salt thereof, or a solvate thereof.

In some embodiments, the cancer, in particular the urothelial carcinoma, can be treated with an FGFR inhibitor wherein the FGFR inhibitor is 3-(2,6-dichloro-3,5-dimethoxy-phenyl)-1-{6-[4-(4 ethyl-piperazin-1-yl)-phenylamino]-pyrimid-4-yl}-methyl-urea (NVP-BGJ398) as described in Int'l Publ. No. WO2006/000420:

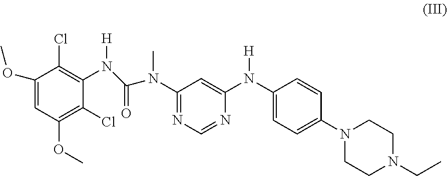 [SMILES: CCN1CCN(c2ccc(Nc3cc(N(C)C(=O)Nc4c(Cl)c(OC)cc(OC)c4Cl)ncn3)cc2)CC1]

including, when chemically possible, any tautomeric or stereochemically isomeric form thereof, and a N-oxide thereof, a pharmaceutically acceptable salt thereof, or a solvate thereof.

In some embodiments, the cancer, in particular the urothelial carcinoma, can be treated with an FGFR inhibitor wherein the FGFR inhibitor is 4-amino-5-fluoro-3-[6-(4-methylpiperazin-1-yl)-1H-benzimidazol-2-yl]-1H-quinolin-2-one (dovitinib) as described in Int't Publ. No. WO2006/127926:

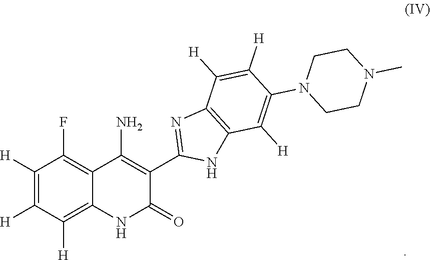 [SMILES: CN1CCN(c2ccc3nc(-c4c(N)c5c(F)cccc5[nH]c4=O)[nH]c3c2)CC1]

including, when chemically possible, any tautomeric or stereochemically isomeric form thereof, and a N-oxide thereof, a pharmaceutically acceptable salt thereof, or a solvate thereof.

In some embodiments, the cancer, in particular the urothelial carcinoma, can be treated with an FGFR inhibitor wherein the FGFR inhibitor is 6-(7-((1-Aminocyclopropyl)-methoxy)-6-methoxyquinolin-4-yloxy)-N-methyl-1-naphthamide (AL3810) (lucitanib; E-3810), as described in Bello, E. et al., E-3810 Is a Potent Dual Inhibitor of VEGFR and FGFR that Exerts Antitumor Activity in Multiple Preclinical Models, Cancer Res Feb. 15, 2011 71(A)1396-1405 and Int'l Publ. No. WO2008/112408:

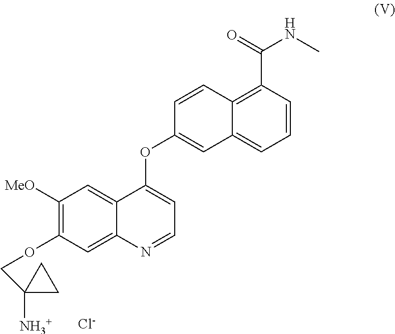 [SMILES: CNC(=O)c1cccc2cc(Oc3ccnc4cc(OCC5([NH3+])CC5)c(OC)cc34)ccc12 ; [Cl-]]

including, when chemically possible, any tautomeric or stereochemically isomeric form thereof, and a N-oxide thereof, a pharmaceutically acceptable salt thereof, or a solvate thereof.

Additional suitable FGFR inhibitors include BAY1163877 (Bayer), BAY1179470 (Bayer), TAS-120 (Taiho), ARQ087 (ArQule), ASP5878 (Astellas), FF284 (Chugai), FP-1039 (GSK/FivePrime), Blueprint, LY-2874455 (Lilly), RG-7444 (Roche), pemigatinib, or any combination thereof, including, when chemically possible, any tautomeric or stereochemical isomeric forms thereof, N-oxides thereof, pharmaceutically acceptable salts thereof, or solvates thereof.

In an embodiment the FGFR inhibitor generally, and erdafitinib more specifically, is administered as a pharmaceutically acceptable salt. In a preferred embodiment the FGFR inhibitor generally, and erdafitinib more specifically, is administered in base form. In an embodiment the FGFR inhibitor generally, and erdafitinib more specifically, is administered as a pharmaceutically acceptable salt in an amount corresponding to 8 mg base equivalent or corresponding to 9 mg base equivalent. In an embodiment the FGFR inhibitor generally, and erdafitinib more specifically, is administered in base form in an amount of 8 mg or 9 mg.

The salts can be prepared by for instance reacting the FGFR inhibitor generally, and erdafitinib more specifically, with an appropriate acid in an appropriate solvent.

Acid addition salts may be formed with acids, both inorganic and organic. Examples of acid addition salts include salts formed with an acid selected from the group consisting of acetic, hydrochloric, hydriodic, phosphoric, nitric, sulphuric, citric, lactic, succinic, maleic, malic, isethionic, fumaric, benzenesulphonic, toluenesulphonic, methanesulphonic (mesylate), ethanesulphonic, naphthalenesulphonic, valeric, acetic, propanoic, butanoic, malonic, glucuronic and lactobionic acids. Another group of acid addition salts includes salts formed from acetic, adipic, ascorbic, aspartic, citric, DL-Lactic, fumaric, gluconic, glucuronic, hippuric, hydrochloric, glutamic, DL-malic, methanesulphonic, sebacic, stearic, succinic and tartaric acids.

In an embodiment, the FGFR inhibitor generally, and erdafitinib more specifically, is administered in the form of a solvate. As used herein, the term “solvate” means a physical association of the FGFR inhibitor generally, and erdafitinib more specifically, with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. The term “solvate” is intended to encompass both solution-phase and isolatable solvates. Non-limiting examples of solvents that may form solvates include water, isopropanol, ethanol, methanol, DMSO, ethyl acetate, acetic acid or ethanolamine and the like.

Solvates are well known in pharmaceutical chemistry. They can be important to the processes for the preparation of a substance (e.g. in relation to their purification, the storage of the substance (e.g. its stability) and the ease of handling of the substance and are often formed as part of the isolation or purification stages of a chemical synthesis. A person skilled in the art can determine by means of standard and long used techniques whether a hydrate or other solvate has formed by the isolation conditions or purification conditions used to prepare a given compound. Examples of such techniques include thermogravimetric analysis (TGA), differential scanning calorimetry (DSC), X-ray crystallography (e.g. single crystal X-ray crystallography or X-ray powder diffraction) and Solid-State NMR (SS-NMR, also known as Magic Angle Spinning NMR or MAS-NMR). Such techniques are as much a part of the standard analytical toolkit of the skilled chemist as NMR, IR, HPLC and MS. Alternatively the skilled person can deliberately form a solvate using crystallization conditions that include an amount of the solvent required for the particular solvate. Thereafter the standard methods described above, can be used to establish whether solvates had formed. Also encompassed are any complexes (e.g. inclusion complexes or clathrates with compounds such as cyclodextrins, or complexes with metals).

Furthermore, the compound may have one or more polymorph (crystalline) or amorphous forms.

The compounds include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. The isotopes may be radioactive or nonradioactive. In one embodiment, the compounds contain no radioactive isotopes. Such compounds are preferred for therapeutic use. In another embodiment, however, the compound may contain one or more radioisotopes. Compounds containing such radioisotopes may be useful in a diagnostic context.

EGFR Inhibitors

Suitable EGFR inhibitors for use in the disclosed methods and uses are provided herein. The EGFR inhibitors may be used alone or in combination with one or more additional EGFR inhibitors, FGFR inhibitors, BRAF inhibitors, CCND1 inhibitors, ARID1A inhibitors, ErbB2 inhibitors, or TERT inhibitors, in the treatment methods and uses described herein.

The term EGFR inhibitor is directed to any one or more agent (drug), compound or molecule that can affect activity and/or expression of a wild type EGFR or an EGFR with one or more of the genetic alterations disclosed herein. In certain embodiments, if one or more EGFR genetic alterations are present in the sample, the cancer, in particular the urothelial carcinoma, can be treated with suitable EGFR inhibitors including the anti-EGFR antibodies cetuximab (Erbitux), pantinumumab (Vectibix), matuzumab, nimotuzumab, small molecule EGFR inhibitors Tarceva (erlotinib), IRESSA (gefitinib), EKB-569 (pelitinib, irreversible EGFR TKI), pan-ErbB and other receptor tyrosine kinase inhibitors lapatinib (EGFR and HER2 inhibitor), pelitinib (EGFR and HER2 inhibitor), vandetanib (ZD6474, ZACTIMA™, EGFR, VEGFR2 and RET TKI), PF00299804 (dacomitinib, irreversible pan-ErbB TKI), CI-1033 (irreversible pan-erbB TKI), afatinib (BIBW2992, irreversible pan-ErbB TKI), AV-412 (dual EGFR and ErbB2 inhibitor), EXEL-7647 (EGFR, ErbB2, GEVGR and EphB4 inhibitor), CO-1686 (irreversible mutant-selective EGFR TKI), AZD9291 (irreversible mutant-selective EGFR TKI), HKI-272 (neratinib, irreversible EGFR/ErbB2 inhibitor), EGFR-targeting short hairpin RNAs (shRNAs), EGFR-targeting small interfering RNAs (siRNAs), and combinations thereof. Each possibility is a separate embodiment.

In some embodiments, if one or more EGFR genetic alterations are present in the sample, the cancer, in particular the urothelial carcinoma, can be treated with a bispecific anti-EGFR/c-Met molecule as described in WO2014081954, the disclosure of which is incorporated herein by reference in its entirety. In certain embodiments, the bispecific anti-EGFR/c-Met molecule is a bispecific anti-EGFR/c-Met antibody. In certain embodiments, the bispecific anti-EGFR/c-Met molecule is amivantamab (also known as JNJ-61186372).

In some embodiments, if one or more EGFR alterations are present in the sample, the cancer, in particular the urothelial carcinoma, can be treated with an EGFR tyrosine kinase inhibitor (TKI). In certain embodiments, the EGFR TKI is osimertinib. In certain embodiments, the EGFR TKI is lazertinib. The structure and synthesis of lazertinib is described in U.S. Pat. No. 9,593,098, the disclosure of which is incorporated herein by reference in its entirety. Lazertinib may also be referred to as N-(5-(4-(4-((dimethylamino)methyl)-3-phenyl-1H-pyrazol-1-yl)pyrimidin-2-ylamino)-4-methoxy-2-morpholinophenyl)acrylamide.

According to particular embodiments, lazertinib is a highly selective and irreversible EGFR TKI with strong inhibitory activity against the single mutation of T790M and dual mutations; e.g., it targets the activating EGFR mutations del19 and L858R, as well as the T790M mutation. In one aspect of the invention, the mutation may be delE746-A750, L858R, or T790M, and it may be dual mutations selected from delE746-A750/T790M or L858R/T790M.

Exemplary EGFR mutations, such as EGFR activating mutations that may be associated with cancer include point mutations, deletion mutations, insertion mutations, inversions or gene amplifications that lead to an increase in at least one biological activity of EGFR, such as elevated tyrosine kinase activity, formation of receptor homodimers and heterodimers, enhanced ligand binding etc. Mutations can be located in any portion of an EGFR gene or regulatory region associated with an EGFR gene and include mutations in exon 18, 19, 20 or 21. Other examples of EGFR activating mutations are known in the art (see e.g., U.S. Pat. Publ. No. US2005/0272083, which is incorporated by reference herein).

In some embodiments, the EGFR mutation is E709K, L718Q, L718V, G719A, G719X, G724X, G724S, 1744T, E746K, L747S, E749Q, A750P, A755V, V765M, C775Y, T790M, L792H, L792V, G796S, G796R, G796C, C797S, T8541, L858P, L858R, L861X, delE746-A750, delE746_T751InsKV, delE746_A750InsHS, delE746_T751InsFPT, delE746_T751InsL, delE746_S752InsIP, delE746_P753InsMS, delE746_T751InsA, delE746_T751InsAPT, delE746_T751InsVA, delE746_S752InsV, delE746_P753InsVS, delE746_K754InsGG, delE746_E749, delE746_E749InsP, delL747_E749, delL747_A750InsP, delL747_T751InsP, delL747_T751InsN, delL747_S752InsPT, delL747_P753InsNS, delL747_S752InsPI, delL747_S752, delL747_P753InsS, delL747_K754, delL747_T751InsS, delL747_T751, delL747_P753InsS, delA750_I759InsPT, delT751_I7591nsT, delS752_I759, delT751_I759InsN, delT751_D761InsNLY, delS752_I759, delR748-P753, delL747-P753insS, delL747-T751, M766_A767InsA, S768_V769InsSVA, P772_H773InsNS, D761_E762InsX, A763_Y764InsX, Y764_Y765 InsX, M766_A767InsX, A767_V768 InsX, S768_V769 InsX, V769_D770 InsX, D770_N771 InsX, N771_P772 InsX, P772_H773 InsX, H773_V774 InsX, V774_C775 InsX, one or more deletions in EGFR exon 20, or one or more insertions in EGFR exon 20, one or more deletions in EGFR exon 19, or one or more insertions in EGFR exon 19, wherein X refers to any of the naturally occurring amino acids and can be one to seven amino acids long.

In some embodiments, the EGFR mutation is the one or more deletions in exon 19 or L858R or any combination thereof. Exemplary exon 19 deletions are delE746-A750, delE746_T751InsKV, delE746_A750InsHS, delE746_T751InsFPT, delE746_T751InsL, delE746_S752InsIP, delE746_P753InsMS, delE746_T751InsA, delE746_T751InsAPT, delE746_T751InsVA, delE746_S752InsV, delE746_P753InsVS, delE746_K754InsGG, delE746_E749, delE746_E749InsP, delL747_E749, delL747_A750InsP, delL747_T751InsP, delL747_T751InsN, delL747_S752InsPT, delL747_P753InsNS, delL747_S752InsPI, delL747_S752, delL747_P753InsS, delL747_K754, delL747_T751InsS, delL747_T751, delL747_P753InsS, delA750_I759InsPT, delT751_I759InsT, delS752_I759, delT751_I759InsN, delT751_D761InsNLY, delS752_I759, delR748-P753 and delL747-P753insS, delL747-T751.

Exemplary c-Met mutations include point mutations, deletion mutations, insertion mutations, inversions or gene amplifications that lead to an increase in at least one biological activity of a c-Met protein, such as elevated tyrosine kinase activity, formation of receptor homodimers and heterodimers, enhanced ligand binding etc. Mutations can be located in any portion of the c-Met gene or regulatory regions associated with the gene, such as mutations in the kinase domain of c-Met. Exemplary c-Met mutations are mutations at residue positions N375, V13, V923, R175, V136, L229, S323, R988, S1058/T1010 and E168, or exon 14 skipping mutations.

In some embodiments, the c-Met mutation is c-Met exon 14 skipping mutation.

Some embodiments described herein provide for an isolated bispecific EGFR/c-Met antibody comprising a HC1, a LC1, a HC2 and a LC2, wherein the HC1 comprises the sequence of SEQ ID NO: 41, the LC1 comprises the sequence of SEQ ID NO: 42, the HC2 comprises the sequence of SEQ ID NO: 43, and the LC2 comprises the sequence of SEQ ID NO: 44. In certain embodiments, the HC1, the LC1, the HC2 and/or the LC2 further comprises 1, 2, 3, 4, 5, 6, 7, 8, 9, 10, 11, 12, 13, 14 or 15 conservative amino acid substitutions.

Bispecific EGFR/c-Met antibodies whose HC1, LC1, HC2 and LC2 amino acid sequences differ insubstantially from those antibodies disclosed herein are encompassed within the scope of the invention. Typically, this involves one or more conservative amino acid substitutions with an amino acid having similar charge, hydrophobic, or stereochemical characteristics in the antigen-binding sites or in the frameworks without adversely altering the properties of the antibody. Conservative substitutions may also be made to improve antibody properties, for example stability or affinity. 1, 2, 3, 4, 5, 6, 7, 8, 9, 10, 11, 12, 13, 14, or 15 amino acid substitutions may be made for example to the VH1, the VL1, the VH2 and/or the VL2. For example, a “conservative amino acid substitution” may involve a substitution of a native amino acid residue with a normative residue such that there is little or no effect on the polarity or charge of the amino acid residue at that position. Furthermore, any native residue in the polypeptide may also be substituted with alanine, as has been previously described for alanine scanning mutagenesis (MacLennan et al., Acta Physiol Scand Suppl 643:55-67, 1998; Sasaki et al., Adv Biophys 35:1-24, 1998). Desired amino acid substitutions may be determined by those skilled in the art at the time such substitutions are desired. For example, amino acid substitutions can be used to identify important residues of the molecule sequence, or to increase or decrease the affinity of the molecules described herein. Exemplary conservative amino acid substitutions are described supra.

The term “bispecific anti-EGFR/c-Met antibody” or “bispecific EGFR/c-Met antibody” as used herein refers to a bispecific antibody having a first domain that specifically binds EGFR and a second domain that specifically binds c-Met. The domains specifically binding EGFR and c-Met are typically VH/VL pairs, and the bispecific anti-EGFR/c-Met antibody is monovalent in terms of binding to EGFR and c-Met.

The term “substituting” or “substituted” or “mutating” or “mutated” as used herein refers to altering, deleting or inserting one or more amino acids or nucleotides in a polypeptide or polynucleotide sequence to generate a variant of that sequence.

“Variant” as used herein refers to a polypeptide or a polynucleotide that differs from a reference polypeptide or a reference polynucleotide by one or more modifications for example, substitutions, insertions or deletions.

The term “specifically binds” or “specific binding” as used herein refers to the ability of a bispecific EGFR/c-Met antibody to bind to a predetermined antigen with a dissociation constant (K_{D}) of about 1×10⁻⁶ M or less, for example about 1×10⁻⁷ M or less, about 1×10⁻⁸ M or less, about 1×10⁻⁹ M or less, about 1×10⁻¹⁰ M or less, about 1×10⁻¹¹ M or less, about 1×10⁻¹² M or less, or about 1×10⁻¹³ M or less. Typically the bispecific EGFR/c-Met antibody binds to a predetermined antigen (i.e. EGFR or c-Met) with a K_{D} that is at least ten fold less than its K_{D} for a nonspecific antigen (for example BSA or casein) as measured by surface plasmon resonance using for example a Proteon Instrument (BioRad). Thus, the bispecific EGFR/c-Met antibody specifically binds to each EGFR and c-Met with a binding affinity (K_{D}) of at least about 1×10⁻⁶ M or less, for example about 1×10⁻⁷ M or less, about 1×10⁻⁸ M or less, about 1×10⁻⁹ M or less, about 1×10⁻¹⁰ M or less, about 1×10⁻¹¹ M or less, about 1×10⁻¹² M or less, or about 1×10⁻¹³ M or less. The bispecific EGFR/c-Met antibody that specifically binds to a predetermined antigen may, however, have cross-reactivity to other related antigens, for example to the same predetermined antigen from other species (homologs).

The term “hepatocyte growth factor receptor” or “c-Met” as used herein refers to the human c-Met having the amino acid sequence shown in SEQ ID NO: 40 or in GenBank Accession No: NP 001120972 and natural variants thereof.

“Blocks binding” or “inhibits binding”, as used herein interchangeably refers to the ability of the bispecific EGFR/c-Met antibody to block or inhibit binding of the EGFR ligand such as EGF to EGFR and/or HGF to c-Met, and encompass both partial and complete blocking/inhibition. The blocking/inhibition of the bispecific EGFR/c-Met antibody reduces partially or completely the normal level of EGFR signaling and/or c-Met signaling when compared to the EGFR ligand binding to EGFR and/or HGF binding to c-Met without blocking or inhibition. The bispecific EGFR/c-Met antibody “blocks binding” of the EGFR ligand such as EGF to EGFR and/or HGF to c-Met when the inhibition is at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100%. Inhibition of binding can be measured using well known methods, for example by measuring inhibition of binding of biotinylated EGF on EGFR expressing A431 cells exposed to the bispecific EGFR/c-Met antibody using FACS, and using methods described herein, or measuring inhibition of binding of biotinylated HGF on c-Met extracellular domain using well known methods and methods described herein.

The term “EGFR signaling” refers to signal transduction induced by EGFR ligand binding to EGFR resulting in autophosphorylation of at least one tyrosine residue in the EGFR. An exemplary EGFR ligand is EGF.

The term “antibodies” as used herein is meant in a broad sense and includes immunoglobulin molecules including polyclonal antibodies, monoclonal antibodies including murine, human, human-adapted, humanized and chimeric monoclonal antibodies, antibody fragments, bispecific or multispecific antibodies, dimeric, tetrameric or multimeric antibodies, and single chain antibodies.

Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species can be assigned to one of two clearly distinct types, namely kappa (κ) and lambda (λ), based on the amino acid sequences of their constant domains.

The term “antibody fragments” refers to a portion of an immunoglobulin molecule that retains the heavy chain and/or the light chain antigen binding site, such as heavy chain complementarity determining regions (HCDR) 1, 2 and 3, light chain complementarity determining regions (LCDR) 1, 2 and 3, a heavy chain variable region (VH), or a light chain variable region (VL). Antibody fragments include a Fab fragment, a monovalent fragment consisting of the VL, VH, CL and CHi domains; a F(ab)2 fragment, a bivalent fragment comprising two Fab fragments linked by a disulfide bridge at the hinge region; a Fd fragment consisting of the VH and CHi domains; a Fv fragment consisting of the VL and VH domains of a single arm of an antibody; a domain antibody (dAb) fragment (Ward et al (1989) Nature 341:544-546), which consists of a VH domain. VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody; described for example in PCT Intl. Publ. Nos. WO1998/44001, WO1988/01649, WO1994/13804, and WO1992/01047. These antibody fragments are obtained using well known techniques known to those of skill in the art, and the fragments are screened for utility in the same manner as are full length antibodies.

The phrase “isolated antibody” refers to an antibody or antibody fragment that is substantially free of other antibodies having different antigenic specificities (e.g., an isolated bispecific antibody specifically binding EGFR and c-Met is substantially free of antibodies that specifically bind antigens other than human EGFR and c-Met). An isolated antibody that specifically binds EGFR and c-Met, however, can have cross-reactivity to other antigens, such as orthologs of human EGFR and/or c-Met, such as Macaca fascicularis (cynomolgus) EGFR and/or c-Met. Moreover, an isolated antibody may be substantially free of other cellular material and/or chemicals.

An antibody variable region consists of a “framework” region interrupted by three “antigen binding sites”. The antigen binding sites are defined using various terms: (i) Complementarity Determining Regions (CDRs), three in the VH (HCDR1, HCDR2, HCDR3), and three in the VL (LCDR1, LCDR2, LCDR3), are based on sequence variability (Wu and Kabat J Exp Med 132:211-50, 1970; Kabat et al Sequences of Proteins of Immunological Interest, 5th Ed. Public Health Service, National Institutes of Health, Bethesda, Md., 1991); (ii) “Hypervariable regions”, “HVR”, or “HV”, three in the VH (H1, H2, H3) and three in the VL (L1, L2, L3), refer to the regions of an antibody variable domains which are hypervariable in structure as defined by Chothia and Lesk (Chothia and Lesk Mol Biol 196:901-17, 1987). Other terms include “IMGT-CDRs” (Lefranc et al., Dev Comparat Immunol 27:55-77, 2003) and “Specificity Determining Residue Usage” (SDRU) (Almagro Mol Recognit 17:132-43, 2004). The International ImMunoGeneTics (IMGT) database (http://www_imgt org) provides a standardized numbering and definition of antigen-binding sites. The correspondence between CDRs, HVs and IMGT delineations is described in Lefranc et al., Dev Comparat Immunol 27:55-77, 2003.

“Chothia residues” as used herein are the antibody VL and VH residues numbered according to Al-Lazikani (Al-Lazikani et al., J Mol Biol 273:927-48, 1997).

“Framework” or “framework sequences” are the remaining sequences of a variable region other than those defined to be antigen binding sites. Because the antigen binding sites can be defined by various terms as described above, the exact amino acid sequence of a framework depends on how the antigen-binding site was defined.

“Humanized antibody” refers to an antibody in which the antigen binding sites are derived from non-human species and the variable region frameworks are derived from human immunoglobulin sequences. Humanized antibodies may include substitutions in the framework regions so that the framework may not be an exact copy of expressed human immunoglobulin or germline gene sequences.

“Human antibody” refers to an antibody having heavy and light chain variable regions in which both the framework and the antigen binding sites are derived from sequences of human origin. If the antibody contains a constant region, the constant region also is derived from sequences of human origin.

Human antibody comprises heavy or light chain variable regions that are “derived from” sequences of human origin if the variable regions of the antibody are obtained from a system that uses human germline immunoglobulin or rearranged immunoglobulin genes. Such systems include human immunoglobulin gene libraries displayed on phage, and transgenic non-human animals such as mice carrying human immunoglobulin loci as described herein. “Human antibody” may contain amino acid differences when compared to the human germline or rearranged immunoglobulin sequences due to for example naturally occurring somatic mutations or intentional introduction of substitutions in the framework or antigen binding sites. Typically, “human antibody” is at least about 80%, 81%, 82%, 83%, 84%, 85%, 86%, 87%, 88%, 89%, 90%, 91%, 92%, 93%, 94%, 95%, 96%, 97%, 98%, 99% or 100% identical in amino acid sequence to an amino acid sequence encoded by a human germline or rearranged immunoglobulin gene. In some cases, “human antibody” may contain consensus framework sequences derived from human framework sequence analyses, for example as described in Knappik et al., J Mol Biol 296:57-86, 2000), or synthetic HCDR3 incorporated into human immunoglobulin gene libraries displayed on phage, for example as described in Shi et al., J Mol Biol 397:385-96, 2010 and Intl. Pat. Publ. No. WO2009/085462). Antibodies in which antigen binding sites are derived from a non-human species are not included in the definition of “human antibody”.

Isolated humanized antibodies may be synthetic. Human antibodies, while derived from human immunoglobulin sequences, may be generated using systems such as phage display incorporating synthetic CDRs and/or synthetic frameworks, or can be subjected to in vitro mutagenesis to improve antibody properties, resulting in antibodies that do not naturally exist within the human antibody germline repertoire in vivo.

The term “recombinant antibody” as used herein, includes all antibodies that are prepared, expressed, created or isolated by recombinant means, such as antibodies isolated from an animal (e.g., a mouse) that is transgenic or transchromosomal for human immunoglobulin genes or a hybridoma prepared therefrom (described further below), antibodies isolated from a host cell transformed to express the antibody, antibodies isolated from a recombinant, combinatorial antibody library, and antibodies prepared, expressed, created or isolated by any other means that involve splicing of human immunoglobulin gene sequences to other DNA sequences, or antibodies that are generated in vitro using Fab arm exchange.

The term “monoclonal antibody” as used herein refers to a preparation of antibody molecules of single molecular composition. A monoclonal antibody composition displays a single binding specificity and affinity for a particular epitope, or in a case of a bispecific monoclonal antibody, a dual binding specificity to two distinct epitopes.

The term “substantially identical” as used herein means that the two antibody variable region amino acid sequences being compared are identical or have “insubstantial differences”. Insubstantial differences are substitutions of 1, 2, 3, 4, 5, 6, 7, 8, 9, 10, 11, 12, 13, 14, or 15 amino acids in an antibody variable region sequence that do not adversely affect antibody properties Amino acid sequences substantially identical to the variable region sequences disclosed herein are within the scope of the invention. In some embodiments, the sequence identity can be about 90%, 91%, 92%, 93%, 94%, 95%, 96%, 97%, 98%, 99% or higher. Percent identity can be determined for example by pairwise alignment using the default settings of the AlignX module of Vector NTI v.9.0.0 (Invitrogen, Carlsbad, Calif.). The protein sequences, in particular those described herein, can be used as a query sequence to perform a search against public or patent databases to, for example, identify related sequences. Exemplary programs used to perform such searches are the XBLAST or BLASTP programs (http_//www_ncbi_nlm/nih_gov), or the GenomeQuest™ (GenomeQuest, Westborough, Mass.) suite using the default settings.

The term “epitope” as used herein means a portion of an antigen to which an antibody specifically binds. Epitopes usually consist of chemically active (such as polar, non-polar or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

BRAF Inhibitors

Suitable BRAF inhibitors for use in the disclosed methods and uses are provided herein. The BRAF inhibitors may be used alone or in combination with one or more additional BRAF inhibitors, FGFR inhibitors, EGFR inhibitors, CCND1 inhibitors, ARID1A inhibitors, ErbB2 inhibitors, or TERT inhibitors, in the treatment methods and uses described herein.

The term BRAF inhibitor is directed to any one or more agent (drug), compound or molecule that can affect activity and/or expression of a wild type BRAF or a BRAF with one or more of the genetic alterations disclosed herein. The inhibitor may be selective or non-selective. In one embodiment, the compounds or agents antagonize BRAF and inhibit a downstream biological effect (e.g., inhibit the phosphorylation of MEK and ERK) that is associated with constitutive BRAF activity. In some embodiments, the inhibitor may exhibit a paradoxical MAPK effect, in which the inhibitor induces increased MAPK activity. In some embodiment, the BRaf inhibitor may include the compound, a derivative thereof, an acceptable salt thereof and/or a solvate thereof. In an embodiment, the BRAF inhibitor is an inhibitor of the BRAF signaling pathway such as mitogen-activated protein kinase kinase (MEK) inhibitors.

In certain embodiments, if one or more BRAF genetic alterations are present in the sample, the cancer, in particular the urothelial carcinoma, can be treated with suitable inhibitors of the BRAF signaling pathway such as mitogen-activated protein kinase kinase (MEK) inhibitors including U0126, 2′-Amino-3′-methoxyflavone, SB2033580 (4-(4′-Fluorophenyl)-2-(4′-methylsulfinylphenyl)-5-(4′-pyridyl)-imidazole), CI-1040 (PD184352), PD325901, GDC 0973 (XL 518), AZD6244 (selumetinib, ARRY-142886), GSK1120212 (trametinib), RDEA119 (refametinib), PD318088, AS703026, AZD8330, TAK-733, CH4987655 (R04987655), MEK-162 (binimetinib), PD98059, and combinations thereof. In certain embodiments, if one or more BRAF genetic alterations are present in the sample, the cancer, in particular the urothelial carcinoma, can be treated with suitable inhibitors of the BRAF including BRAF-targeting short hairpin RNAs (shRNAs), BRAF-targeting small interfering RNAs (siRNAs), and combinations thereof. Each possibility is a separate embodiment.

In addition, compounds that inhibit oncogenic BRAF (BRAF or mutated BRAF) expression or activity may be readily identified using screening methods well known to those of skill in the art (e.g. see US 2008/0072337). In one embodiment, compounds identified by the screening methods bind specifically to a BRAF nucleic acid or to BRAF polypeptide. In vivo or cell culture assays may be used to determine whether a test compound functions as an antagonist to inhibit BRAF in cells.

CCND1 Inhibitors

Suitable CCND1 inhibitors for use in the disclosed methods and uses are provided herein. The CCND1 inhibitors may be used alone or in combination with one or more additional CCND1 inhibitors, FGFR inhibitors, EGFR inhibitors, BRAF inhibitors, ARID1A inhibitors, ErbB2 inhibitors, or TERT inhibitors, in the treatment methods and uses described herein.

The term CCND1 inhibitor is directed to any one or more agent (drug), compound or molecule that can affect activity and/or expression of a wild type CCND1 or a CCND1 with one or more of the genetic alterations disclosed herein. In some embodiments, if one or more CCND1 genetic alterations are present in the sample, the cancer, in particular the urothelial carcinoma, can be treated with suitable CCND1 inhibitors including Indirubin, Arcyriaflavin A, NSC 625987, Fascaplysin, Indirubin-5-sulfonic acid sodium salt, indolo[6,7-a]pyrrolo[3,4-c]carbazole, CCND1-targeting short hairpin RNAs (shRNAs), CCND1-targeting small interfering RNAs (siRNAs), and combinations thereof. Each possibility is a separate embodiment.

ErbB2 Inhibitors

Suitable ErbB2 inhibitors for use in the disclosed methods and uses are provided herein. The ErbB2 inhibitors may be used alone or in combination with one or more additional ErbB2 inhibitors, FGFR inhibitors, EGFR inhibitors, BRAF inhibitors, ARID1A inhibitors, CCND1 inhibitors, or TERT inhibitors, in the treatment methods and uses described herein.

The term ErbB2 inhibitor is directed to any one or more agent (drug), compound or molecule that can affect activity and/or expression of a wild type ErbB2 or an ErbB2 with one or more of the genetic alterations disclosed herein. In certain embodiments, the ErbB2 inhibitor is a compound or an agent that is effective in inhibiting ErbB2 activity by either inhibiting ErbB2 phosphorylation (i.e., inhibiting ErbB2 activation, blocking ErbB2 kinase activity and downstream signaling) or causing a reduction in the ErbB2 protein level.

In certain embodiments, if one or more ErbB2 genetic alterations are present in the sample, the cancer, in particular the urothelial carcinoma, can be treated with suitable ErbB2 inhibitors or ErbB2 receptor inhibitors including pertuzumab, trastuzumab (Herceptin), dacomitinib, ErbB2 antibodies as described in WO-2012162561, neratinib, allitinib tosylate, poziotinib, CUDC-101 (Curis), BT-2111 (biOsasis), margetuximab, Exelixis, NT-004 or NT-113 (Jiangsu Kanion Pharmaceutical Co Ltd), S-222611 (Shionogi & Co Ltd), AG879, Mubritinib, AC-480 (Bristol-Myers Squibb Co), sapitinib, MM-111 (Merrimack Pharmaceuticals Inc), PR-610 (University of Auckland), cipatinib trastuzumab-duocarmycin, Prolanta, varlitinib, kahalalide F, TrasGEX, masoprocol, ARRY-380 (Array BioPharma), erbicinumab, HuMax-Her2, CP-724714 (Pfizer), COVA-208 (Covagen), lapatinib and pazopanib, AEE-788 (Novartis), canertinib, pelitinib, BMS-690514 (Bristol-Meyers Squibb), afatinib, dacomitinib, AV-412, EXEL-7647, HKI-272 (neratinib), cetuximab (Erbitux), panitumumab (Vectibix), ErbB2-targeting short hairpin RNAs (shRNAs), ErbB2-targeting small interfering RNAs (siRNAs), and combinations thereof. Each possibility is a separate embodiment.

TERT Inhibitors

Suitable TERT inhibitors for use in the disclosed methods and uses are provided herein. The TERT inhibitors may be used alone or in combination with one or more additional TERT inhibitors, FGFR inhibitors, EGFR inhibitors, BRAF inhibitors, ARID1A inhibitors, ErbB2 inhibitors, or CCND1 inhibitors, in the treatment methods and uses described herein.

The term TERT inhibitor is directed to any one or more agent (drug), compound or molecule that can affect activity and/or expression of a wild type TERT or a TERT with one or more of the genetic alterations disclosed herein. In certain embodiments, if one or more TERT genetic alterations are present in the sample, the cancer, in particular the urothelial carcinoma, can be treated with suitable TERT inhibitors including TERT-targeting short hairpin RNAs (shRNAs), TERT-targeting small interfering RNAs (siRNAs), and combinations thereof. Each possibility is a separate embodiment.

ARID1A Inhibitors

Suitable ARID1A inhibitors for use in the disclosed methods and uses are provided herein. The ARID1A inhibitors may be used alone or in combination with one or more additional ARID1A inhibitors, FGFR inhibitors, EGFR inhibitors, BRAF inhibitors, TERT inhibitors, ErbB2 inhibitors, or CCND1 inhibitors, in the treatment methods and uses described herein.

The term ARID1A inhibitor is directed to any one or more agent (drug), compound or molecule that can affect activity and/or expression of a wild type ARID1A or an ARID1A with one or more of the genetic alterations disclosed herein. In certain embodiments, if one or more ARID1A genetic alterations are present in the sample, the cancer, in particular the urothelial carcinoma, can be treated with suitable ARID1A inhibitors including ARID1A-targeting short hairpin RNAs (shRNAs), ARID1A-targeting small interfering RNAs (siRNAs), and combinations thereof. In certain embodiments, if one or more ARID1A genetic alterations are present in the sample, the cancer, in particular the urothelial carcinoma, can be treated with suitable inhibitors of the bromodomain and extra terminal domain (BET) family of proteins, in particular BRD2 inhibitors, or with immune checkpoint inhibitors, in particular PD-L1 inhibitors. In certain embodiments, the BET inhibitor is JQ1 or iBET-762. Each possibility is a separate embodiment.

Methods of Treatment Compounds for Use

In certain embodiments of the disclosed methods and uses, the cancer is urothelial carcinoma. In some embodiments, the urothelial carcinoma is locally advanced or metastatic. In certain embodiments the cancer is mUC. In certain embodiments, the patient is a high-risk patient, in particular a high-risk patient with metastatic or surgically unresectable urothelial cancer, in particular metastatic or surgically unresectable urothelial cancer harboring select FGFR genetic alterations (FGFR translocations or mutations), in particular FGFR genetic alterations as defined herein in addition to at least one EGFR, CCND1, BRAF, ARID1A, ErbB2 or TERT genetic alteration as defined herein. A high-risk patient is a patient meeting one or more of the following criteria: age≥75 years; ECOG PS 2; hemoglobin<10 g/dL; visceral metastases, in particular of the liver, lung and/or bone; and 2 or 3 Bellmunt risk factors. In an embodiment the hemoglobin level is measured in whole blood.

In certain embodiments of the disclosed methods and uses, the patient is resistant to treatment with erdafitinib or has acquired resistance to treatment with erdafitinib.

FGFR Combination Therapies

Described herein are methods of treating cancer generally, and mUC more specifically, comprising, consisting of, or consisting essential of, administering an FGFR inhibitor in combination with a second FGFR inhibitor to a patient in need of cancer treatment generally, and mUC treatment more specifically, wherein the patient harbors at least one FGFR2 genetic alteration or FGFR3 genetic alteration and at least one FGFR1 genetic alteration.

Also described herein are methods of treating cancer generally, and mUC more specifically, in a patient comprising, consisting of, or consisting essential of: (a) evaluating a biological sample from the patient for the presence of at least one FGFR2 genetic alteration or FGFR3 genetic alteration and at least one FGFR1 genetic alteration; and (b) treating the patient with an FGFR inhibitor in combination with a second FGFR inhibitor if the at least one FGFR2 genetic alteration or FGFR3 genetic alteration and the at least one FGFR1 genetic alteration are present in the sample.

Still further described herein are two or more FGFR inhibitors for use in the treatment of cancer generally, and mUC more specifically, in a patient who harbors at least one FGFR2 genetic alteration or FGFR3 genetic alteration and at least one FGFR1 genetic alteration.

Also described herein are uses of FGFR inhibitors for the manufacture of a medicament for the treatment of cancer generally, and mUC more specifically, in a patient who harbors at least one FGFR2 genetic alteration or FGFR3 genetic alteration and at least one FGFR1 genetic alteration, wherein the FGFR inhibitor is to be used in combination with a second FGFR inhibitor.

In certain embodiments, administration of the FGFR inhibitor in combination with a second FGFR inhibitor provides improved anti-tumor activity, as measured by OS or PFS, relative to a patient or patient population with cancer generally, and mUC more specifically, who was not receiving treatment with an FGFR inhibitor in combination with a second FGFR inhibitor. In certain embodiments, administration of the FGFR inhibitor in combination with a second FGFR inhibitor provides improved anti-tumor activity, as measured by OS, relative to a patient or patient population with cancer generally, and mUC more specifically, who was not receiving treatment with an FGFR inhibitor in combination with a second inhibitor. In certain embodiments, administration of the FGFR inhibitor in combination with a second inhibitor provides improved anti-tumor activity, as measured by PFS, relative to a patient or patient population with cancer generally, and mUC more specifically, who was not receiving treatment with an FGFR inhibitor in combination with a second FGFR inhibitor.

In certain embodiments, the improvement in anti-tumor activity is relative to treatment with only one FGFR inhibitor. In certain embodiments, the improvement in anti-tumor activity is relative to treatment with placebo. In certain embodiments, the improvement in anti-tumor activity is relative to no treatment. In certain embodiments, the improvement in anti-tumor activity is relative to standard of care. In certain embodiments, the improvement in anti-tumor activity is relative to a patient population without mUC.

Further described herein are methods of predicting duration of PFS in a patient, in particular a human patient, having cancer generally, and mUC more specifically, in particular in a patient on treatment with an FGFR inhibitor, the method comprising evaluating a biological sample from the patient for the presence of at least one FGFR2 genetic alteration or FGFR3 genetic alteration and at least one FGFR1 genetic alteration, wherein the presence of at least one FGFR2 genetic alteration or FGFR3 genetic alteration and at least one FGFR1 genetic alteration indicates a shorter duration of PFS, relative to a patient, in particular a human patient, having cancer generally, and mUC more specifically, who does not harbor at least one FGFR1 genetic alteration, or relative to a patient, in particular a human patient, having cancer generally, and mUC more specifically, who does not harbor at least one FGFR2 genetic alteration or FGFR3 genetic alteration and at least one FGFR1 genetic alteration.

Still further described herein are methods of predicting duration of OS in a patient, in particular a human patient, having cancer generally, and mUC more specifically, in particular in a patient on treatment with ant FGFR inhibitor, the method comprising evaluating a biological sample from the patient for the presence of at least one FGFR2 genetic alteration or FGFR3 genetic alteration and at least one FGFR1 genetic alteration, wherein the presence of at least one FGFR2 genetic alteration or FGFR3 genetic alteration and at least one FGFR1 genetic alteration indicates a shorter duration of OS relative to a patient, in particular a human patient, having cancer generally, and mUC more specifically, who does not harbor at least one FGFR1 genetic alteration, or relative to a patient, in particular a human patient, having cancer generally, and mUC more specifically, who does not harbor at least one FGFR2 genetic alteration or FGFR3 genetic alteration and at least one FGFR1 genetic alteration.

Also described herein are methods of improving OS in a patient with cancer generally, and mUC more specifically, relative to a patient with cancer generally, and mUC more specifically, who was not receiving treatment with an FGFR inhibitor in combination with a second FGFR inhibitor, said method comprising providing to said patient an FGFR inhibitor in combination with a second FGFR inhibitor, wherein said patient harbors at least one FGFR2 genetic alteration or FGFR3 genetic alteration and at least one FGFR1 genetic alteration.

Further described herein are methods of improving PFS in a patient with cancer generally, and mUC more specifically, relative to a patient with cancer generally, and mUC more specifically, who was not receiving treatment with an FGFR inhibitor in combination with a second FGFR inhibitor, said method comprising providing to said patient an FGFR inhibitor in combination with a second FGFR inhibitor, wherein said patient harbors at least one FGFR2 genetic alteration or FGFR3 genetic alteration and at least one FGFR1 genetic alteration.

Said methods and uses also encompass administration of at least one, two, three or four FGFR inhibitors to a patient that has been diagnosed with cancer generally, or urothelial carcinoma more specifically. In addition to administration of FGFR inhibitors, as described herein, said methods and uses encompass administration of at least one, two three, or four EGFR, CCND1, BRAF, ARID1A, ErbB2, or TERT inhibitors, or any combination thereof, to a patient who harbors at least one EGFR, CCND1, BRAF, ARID1A, ErbB2, or TERT genetic alterations, or any combination thereof, respectively. In certain embodiments, in addition to administration of FGFR inhibitors, as described herein, said methods and uses also encompass administration of at least one, two, three, or four CCND1 inhibitors if the patient also harbors at least one CCND1 genetic alteration. In certain embodiments, in addition to administration of FGFR inhibitors as described herein, said methods and uses also encompass administration of at least one, two, three, or four EGFR inhibitors if the patient also harbors at least one EGFR genetic alteration. In certain embodiments, in addition to administration of FGFR inhibitors as described herein, said methods and uses also encompass administration of at least one, two, three, or four BRAF inhibitors if the patient also harbors at least one BRAF genetic alteration. In certain embodiments, in addition to administration of FGFR inhibitors as described herein, said methods and uses also encompass administration of at least one, two, three, or four ARID1A inhibitors if the patient also harbors at least one ARID1A genetic alteration. In certain embodiments, in addition to administration of FGFR inhibitors as described herein, said methods and uses also encompass administration of at least one, two, three, or four ErbB2 inhibitors if the patient also harbors at least one ErbB2 genetic alteration. In certain embodiments, in addition to administration of FGFR inhibitors as described herein, said methods and uses also encompass administration of at least one, two, three, or four TERT inhibitors if the patient also harbors at least one TERT genetic alteration.

FGFR and EGFR Combination Therapies

Described herein are methods of treating cancer generally, and mUC more specifically, comprising, consisting of, or consisting essential of, administering an FGFR inhibitor in combination with an EGFR inhibitor to a patient in need of cancer treatment generally, and mUC treatment more specifically, wherein the patient harbors at least one FGFR2 genetic alteration or FGFR3 genetic alteration and at least one EGFR genetic alteration.

Also described herein are methods of treating cancer generally, and mUC more specifically, in a patient comprising, consisting of, or consisting essential of: (a) evaluating a biological sample from the patient for the presence of at least one FGFR2 genetic alteration or FGFR3 genetic alteration and at least one EGFR genetic alteration; and (b) treating the patient with an FGFR inhibitor in combination with an EGFR inhibitor if the at least one FGFR2 genetic alteration or FGFR3 genetic alteration and the at least one EGFR genetic alteration are present in the sample.

Still further described herein are an FGFR inhibitor and an EGFR inhibitor for use in the treatment of cancer generally, and mUC more specifically, in a patient who harbors at least one FGFR2 genetic alteration or FGFR3 genetic alteration and at least one EGFR genetic alteration. Also described herein is an FGFR inhibitor for use in the treatment of cancer generally, and mUC more specifically, in a patient who harbors at least one FGFR2 genetic alteration or FGFR3 genetic alteration and at least one EGFR genetic alteration, wherein the FGFR inhibitor is to be used in combination with an EGFR inhibitor. Also described herein is an EGFR inhibitor for use in the treatment of cancer generally, and mUC more specifically, in a patient who harbors at least one FGFR2 genetic alteration or FGFR3 genetic alteration and at least one EGFR genetic alteration, wherein the EGFR inhibitor is to be used in combination with an FGFR inhibitor.

Also described herein are uses of an FGFR inhibitor for the manufacture of a medicament for the treatment of cancer generally, and mUC more specifically, in a patient who harbors at least one FGFR2 genetic alteration or FGFR3 genetic alteration and at least one EGFR genetic alteration, wherein the FGFR inhibitor is to be used in combination with an EGFR inhibitor. Also described herein are uses of an EGFR inhibitor for the manufacture of a medicament for the treatment of cancer generally, and mUC more specifically, in a patient who harbors at least one FGFR2 genetic alteration or FGFR3 genetic alteration and at least one EGFR genetic alteration, wherein the EGFR inhibitor is to be used in combination with an FGFR inhibitor.

In certain embodiments, administration of the FGFR inhibitor in combination with an EGFR inhibitor provides improved anti-tumor activity, as measured by OS or PFS, relative to a patient or patient population with cancer generally, and mUC more specifically, who was not receiving treatment with an FGFR inhibitor in combination with an EGFR inhibitor. In certain embodiments, administration of the FGFR inhibitor in combination with an EGFR inhibitor provides improved anti-tumor activity, as measured by OS, relative to a patient or patient population with cancer generally, and mUC more specifically, who was not receiving treatment with an FGFR inhibitor in combination with an EGFR inhibitor. In certain embodiments, administration of the FGFR inhibitor in combination with an EGFR inhibitor provides improved anti-tumor activity, as measured by PFS, relative to a patient or patient population with cancer generally, and mUC more specifically, who was not receiving treatment with an FGFR inhibitor in combination with an EGFR inhibitor.

In certain embodiments, the improvement in anti-tumor activity is relative to treatment with an FGFR inhibitor. In certain embodiments, the improvement in anti-tumor activity is relative to treatment with placebo. In certain embodiments, the improvement in anti-tumor activity is relative to no treatment. In certain embodiments, the improvement in anti-tumor activity is relative to standard of care. In certain embodiments, the improvement in anti-tumor activity is relative to a patient population without mUC.

To assess overall response or future progression, the overall tumor burden at baseline may be estimated and used as a comparator for subsequent measurements. Measurable disease is defined by the presence of at least one measurable lesion.

Further described herein are methods of predicting duration of PFS in a patient, in particular a human patient, having cancer generally, and mUC more specifically, in particular a patient on treatment with an FGFR inhibitor, the method comprising evaluating a biological sample from the patient for the presence of at least one FGFR2 genetic alteration or FGFR3 genetic alteration and at least one EGFR genetic alteration, wherein the presence of at least one FGFR2 genetic alteration or FGFR3 genetic alteration and at least one EGFR genetic alteration indicates a shorter duration of PFS, relative to a patient, in particular a human patient, having cancer generally, and mUC more specifically, who does not harbor at least one EGFR genetic alteration, or relative to a patient, in particular a human patient, having cancer generally, and mUC more specifically, who does not harbor at least one FGFR2 genetic alteration or FGFR3 genetic alteration and at least one EGFR genetic alteration.

Still further described herein are methods of predicting duration of OS in a patient, in particular a human patient, having cancer generally, and mUC more specifically, in particular in a patient on treatment with an FGFR inhibitor, the method comprising evaluating a biological sample from the patient for the presence of at least one FGFR2 genetic alteration or FGFR3 genetic alteration and at least one EGFR genetic alteration, wherein the presence of at least one FGFR2 genetic alteration or FGFR3 genetic alteration and at least one EGFR genetic alteration indicates a shorter duration of OS relative to a patient, in particular a human patient, having cancer generally, and mUC more specifically, who does not harbor at least one EGFR genetic alteration, or relative to a patient, in particular a human patient, having cancer generally, and mUC more specifically, who does not harbor at least one FGFR2 genetic alteration or FGFR3 genetic alteration and at least one EGFR genetic alteration.

Also described herein are methods of improving OS in a patient with cancer generally, and mUC more specifically, relative to a patient with cancer generally, and mUC more specifically, who was not receiving treatment with an FGFR inhibitor in combination with an EGFR inhibitor, said method comprising providing to said patient an FGFR inhibitor in combination with an EGFR inhibitor, wherein said patient harbors at least one FGFR2 genetic alteration or FGFR3 genetic alteration and at least one EGFR genetic alteration.

Also described herein is an FGFR inhibitor for use in improving OS in a patient with cancer generally, and mUC more specifically, relative to a patient with cancer generally, and mUC more specifically, who was not receiving treatment with an FGFR inhibitor in combination with an EGFR inhibitor, wherein the FGFR inhibitor is to be used in combination with an EGFR inhibitor and wherein said patient harbors at least one FGFR2 genetic alteration or FGFR3 genetic alteration and at least one EGFR genetic alteration. Also described herein is an EGFR inhibitor for use in improving OS in a patient with cancer generally, and mUC more specifically, relative to a patient with cancer generally, and mUC more specifically, who was not receiving treatment with an EGFR inhibitor in combination with an FGFR inhibitor, wherein the EGFR inhibitor is to be used in combination with an FGFR inhibitor and wherein said patient harbors at least one FGFR2 genetic alteration or FGFR3 genetic alteration and at least one EGFR genetic alteration.

Further described herein are methods of improving PFS in a patient with cancer generally, and mUC more specifically, relative to a patient with cancer generally, and mUC more specifically, who was not receiving treatment with an FGFR inhibitor in combination with an EGFR inhibitor, said method comprising providing to said patient an FGFR inhibitor in combination with an EGFR inhibitor, wherein said patient harbors at least one FGFR2 genetic alteration or FGFR3 genetic alteration and at least one EGFR genetic alteration.

Also described herein is an FGFR inhibitor for use in improving PFS in a patient with cancer generally, and mUC more specifically, relative to a patient with cancer generally, and mUC more specifically, who was not receiving treatment with an FGFR inhibitor in combination with an EGFR inhibitor, wherein the FGFR inhibitor is to be used in combination with an EGFR inhibitor and wherein said patient harbors at least one FGFR2 genetic alteration or FGFR3 genetic alteration and at least one EGFR genetic alteration. Also described herein is an EGFR inhibitor for use in improving PFS in a patient with cancer generally, and mUC more specifically, relative to a patient with cancer generally, and mUC more specifically, who was not receiving treatment with an EGFR inhibitor in combination with an FGFR inhibitor, wherein the EGFR inhibitor is to be used in combination with an FGFR inhibitor and wherein said patient harbors at least one FGFR2 genetic alteration or FGFR3 genetic alteration and at least one EGFR genetic alteration.

Said methods and uses also encompass administration of at least one, two, three or four FGFR in combination with at least one, two, three or four EGFR inhibitors to a patient that has been diagnosed with cancer generally, and mUC more specifically. In addition to administration of FGFR and EGFR inhibitors, as described herein, said methods and uses encompass administration of at least one, two three, or four CCND1, BRAF, ARID1A, ErbB2, or TERT inhibitors, or any combination thereof, to a patient who harbors at least one CCND1, BRAF, ARID1A, ErbB2, or TERT genetic alterations, or any combination thereof, respectively. In certain embodiments, in addition to administration of FGFR and EGFR inhibitors, as described herein, said methods and uses also encompass administration of at least one, two, three, or four CCND1 inhibitors if the patient also harbors at least one CCND1 genetic alteration. In certain embodiments, in addition to administration of FGFR and EGFR inhibitors, as described herein, said methods and uses also encompass administration of at least one, two, three, or four BRAF inhibitors if the patient also harbors at least one BRAF genetic alteration. In certain embodiments, in addition to administration of FGFR and EGFR inhibitors, as described herein, said methods and uses also encompass administration of at least one, two, three, or four ARID1A inhibitors if the patient also harbors at least one ARID1A genetic alteration. In certain embodiments, in addition to administration of FGFR and EGFR inhibitors, as described herein, said methods and uses also encompass administration of at least one, two, three, or four ErbB2 inhibitors if the patient also harbors at least one ErbB2 genetic alteration. In certain embodiments, in addition to administration of FGFR and EGFR inhibitors, as described herein, said methods and uses also encompass administration of at least one, two, three, or four TERT inhibitors if the patient also harbors at least one TERT genetic alteration.

In certain embodiments, in addition to administration of FGFR and EGFR inhibitors, as described herein, said methods and uses also encompass administration of at least one, two, three, or four CCND1 inhibitors if the patient also harbors at least one CCND1 genetic alteration and at least one, two, three, or four BRAF inhibitors if the patient also harbors at least one BRAF genetic alteration.

FGFR and CCND1 Combination Therapies

Described herein are methods of treating cancer generally, and mUC more specifically, comprising, consisting of, or consisting essential of, administering an FGFR inhibitor in combination with a CCND1 inhibitor to a patient in need of cancer treatment generally, and mUC treatment more specifically, wherein the patient harbors at least one FGFR2 genetic alteration or FGFR3 genetic alteration and at least one CCND1 genetic alteration.

Also described herein are methods of treating cancer generally, and mUC more specifically, in a patient comprising, consisting of, or consisting essential of: (a) evaluating a biological sample from the patient for the presence of at least one FGFR2 genetic alteration or FGFR3 genetic alteration and at least one CCND1 genetic alteration; and (b) treating the patient with an FGFR inhibitor in combination with a CCND1 inhibitor if the at least one FGFR2 genetic alteration or FGFR3 genetic alteration and the at least one CCND1 genetic alteration are present in the sample.

Still further described herein are an FGFR inhibitor and a CCND1 inhibitor for use in the treatment of cancer generally, and mUC more specifically, in a patient who harbors at least one FGFR2 genetic alteration or FGFR3 genetic alteration and at least one CCND1 genetic alteration. Also described herein is an FGFR inhibitor for use in the treatment of cancer generally, and mUC more specifically, in a patient who harbors at least one FGFR2 genetic alteration or FGFR3 genetic alteration and at least one CCND1 genetic alteration, wherein the FGFR inhibitor is to be used in combination with a CCND1 inhibitor. Also described herein is a CCND1 inhibitor for use in the treatment of cancer generally, and mUC more specifically, in a patient who harbors at least one FGFR2 genetic alteration or FGFR3 genetic alteration and at least one CCND1 genetic alteration, wherein the CCND1 inhibitor is to be used in combination with an FGFR inhibitor.

Also described herein are uses of an FGFR inhibitor for the manufacture of a medicament for the treatment of cancer generally, and mUC more specifically, in a patient who harbors at least one FGFR2 genetic alteration or FGFR3 genetic alteration and at least one CCND1 genetic alteration, wherein the FGFR inhibitor is to be used in combination with a CCND1 inhibitor. Also described herein are uses of a CCND1 inhibitor for the manufacture of a medicament for the treatment of cancer generally, and mUC more specifically, in a patient who harbors at least one FGFR2 genetic alteration or FGFR3 genetic alteration and at least one CCND1 genetic alteration, wherein the CCND1 inhibitor is to be used in combination with an FGFR inhibitor.

In certain embodiments, administration of the FGFR inhibitor in combination with a CCND1 inhibitor provides improved anti-tumor activity, as measured by OS or PFS, relative to a patient or patient population with cancer generally, and mUC more specifically, who was not receiving treatment with an FGFR inhibitor in combination with a CCND1 inhibitor. In certain embodiments, administration of the FGFR inhibitor in combination with a CCND1 inhibitor provides improved anti-tumor activity, as measured by OS, relative to a patient or patient population with cancer generally, and mUC more specifically, who was not receiving treatment with an FGFR inhibitor in combination with a CCND1 inhibitor. In certain embodiments, administration of the FGFR inhibitor in combination with a CCND1 inhibitor provides improved anti-tumor activity, as measured by PFS, relative to a patient or patient population with cancer generally, and mUC more specifically, who was not receiving treatment with an FGFR inhibitor in combination with a CCND1 inhibitor.

In certain embodiments, the improvement in anti-tumor activity is relative to treatment with an FGFR inhibitor. In certain embodiments, the improvement in anti-tumor activity is relative to treatment with placebo. In certain embodiments, the improvement in anti-tumor activity is relative to no treatment. In certain embodiments, the improvement in anti-tumor activity is relative to standard of care. In certain embodiments, the improvement in anti-tumor activity is relative to a patient population without cancer generally, and mUC more specifically.

Further described herein are methods of predicting duration of PFS in a patient, in particular a human patient, having cancer generally, and mUC more specifically, in particular in a patient on treatment with an FGFR inhibitor, the method comprising evaluating a biological sample from the patient for the presence of at least one FGFR2 genetic alteration or FGFR3 genetic alteration and at least one CCND1 genetic alteration, wherein the presence of at least one FGFR2 genetic alteration or FGFR3 genetic alteration and at least one CCND1 genetic alteration indicates a shorter duration of PFS, relative to a patient, in particular a human patient, having cancer generally, and mUC more specifically, who does not harbor at least one CCND1 genetic alteration, or relative to a patient, in particular a human patient, having cancer generally, and mUC more specifically, who does not harbor at least one FGFR2 genetic alteration or FGFR3 genetic alteration and at least one CCND1 genetic alteration.

Still further described herein are methods of predicting duration of OS in a patient, in particular a human patient, having cancer generally, and mUC more specifically, in particular in a patient on treatment with an FGFR inhibitor, the method comprising evaluating a biological sample from the patient for the presence of at least one FGFR2 genetic alteration or FGFR3 genetic alteration and at least one CCND1 genetic alteration, wherein the presence of at least one FGFR2 genetic alteration or FGFR3 genetic alteration and at least one CCND1 genetic alteration indicates a shorter duration of OS relative to a patient, in particular a human patient, having cancer generally, and mUC more specifically, who does not harbor at least one CCND1 genetic alteration, or relative to a patient, in particular a human patient, having cancer generally, and mUC more specifically, who does not harbor at least one FGFR2 genetic alteration or FGFR3 genetic alteration and at least one CCND1 genetic alteration.

Also described herein are methods of improving OS in a patient with cancer generally, and mUC more specifically, relative to a patient with cancer generally, and mUC more specifically, who was not receiving treatment with an FGFR inhibitor in combination with a CCND1 inhibitor, said method comprising providing to said patient an FGFR inhibitor in combination with a CCND1 inhibitor, wherein said patient harbors at least one FGFR2 genetic alteration or FGFR3 genetic alteration and at least one CCND1 genetic alteration.

Also described herein is an FGFR inhibitor for use in improving OS in a patient with cancer generally, and mUC more specifically, relative to a patient with cancer generally, and mUC more specifically, who was not receiving treatment with an FGFR inhibitor in combination with a CCND1 inhibitor, wherein the FGFR inhibitor is to be used in combination with a CCDN1 inhibitor and wherein said patient harbors at least one FGFR2 genetic alteration or FGFR3 genetic alteration and at least one CCND1 genetic alteration. Also described herein is a CCND1 inhibitor for use in improving OS in a patient with cancer generally, and mUC more specifically, relative to a patient with cancer generally, and mUC more specifically, who was not receiving treatment with a CCND1 inhibitor in combination with an FGFR inhibitor, wherein the CCND1 inhibitor is to be used in combination with an FGFR inhibitor and wherein said patient harbors at least one FGFR2 genetic alteration or FGFR3 genetic alteration and at least one CCND1 genetic alteration.

Further described herein are methods of improving PFS in a patient with cancer generally, and mUC more specifically, relative to a patient with cancer generally, and mUC more specifically, who was not receiving treatment with an FGFR inhibitor in combination with a CCND1 inhibitor, said method comprising providing to said patient an FGFR inhibitor in combination with a CCND1 inhibitor, wherein said patient harbors at least one FGFR2 genetic alteration or FGFR3 genetic alteration and at least one CCND1 genetic alteration.

Also described herein is an FGFR inhibitor for use in improving PFS in a patient with cancer generally, and mUC more specifically, relative to a patient with cancer generally, and mUC more specifically, who was not receiving treatment with an FGFR inhibitor in combination with a CCND1 inhibitor, wherein the FGFR inhibitor is to be used in combination with a CCDN1 inhibitor and wherein said patient harbors at least one FGFR2 genetic alteration or FGFR3 genetic alteration and at least one CCND1 genetic alteration. Also described herein is a CCND1 inhibitor for use in improving PFS in a patient with cancer generally, and mUC more specifically, relative to a patient with cancer generally, and mUC more specifically, who was not receiving treatment with a CCND1 inhibitor in combination with an FGFR inhibitor, wherein the CCND1 inhibitor is to be used in combination with an FGFR inhibitor and wherein said patient harbors at least one FGFR2 genetic alteration or FGFR3 genetic alteration and at least one CCND1 genetic alteration.

Said methods and uses also encompass administration of at least one, two, three or four FGFR in combination with at least one, two, three or four CCND1 inhibitors to a patient that has been diagnosed with cancer generally, and mUC more specifically. In addition to administration of FGFR and CCND1 inhibitors, as described herein, said methods and uses encompass administration of at least one, two three, or four EGFR, BRAF, ARID1A, ErbB2, or TERT inhibitors, or any combination thereof, to a patient who harbors at least one EGFR, BRAF, ARID1A, ErbB2, or TERT genetic alterations, or any combination thereof, respectively. In certain embodiments, in addition to administration of FGFR and CCND1 inhibitors, as described herein, said methods and uses also encompass administration of at least one, two, three, or four EGFR inhibitors if the patient also harbors at least one EGFR genetic alteration. In certain embodiments, in addition to administration of FGFR and CCND1 inhibitors, as described herein, said methods and uses also encompass administration of at least one, two, three, or four BRAF inhibitors if the patient also harbors at least one BRAF genetic alteration. In certain embodiments, in addition to administration of FGFR and CCND1 inhibitors, as described herein, said methods and uses also encompass administration of at least one, two, three, or four ARID1A inhibitors if the patient also harbors at least one ARID1A genetic alteration. In certain embodiments, in addition to administration of FGFR and CCND1 inhibitors, as described herein, said methods and uses also encompass administration of at least one, two, three, or four ErbB2 inhibitors if the patient also harbors at least one ErbB2 genetic alteration. In certain embodiments, in addition to administration of FGFR and CCND1 inhibitors, as described herein, said methods and uses also encompass administration of at least one, two, three, or four TERT inhibitors if the patient also harbors at least one TERT genetic alteration.

In certain embodiments, in addition to administration of FGFR and CCND1 inhibitors, as described herein, said methods and uses also encompass administration of at least one, two, three, or four EGFR inhibitors if the patient also harbors at least one EGFR genetic alteration and at least one, two, three, or four BRAF inhibitors if the patient also harbors at least one BRAF genetic alteration.

FGFR and BRAF Combination Therapies

Described herein are methods of treating cancer generally, and mUC more specifically, comprising, consisting of, or consisting essential of, administering an FGFR inhibitor in combination with a BRAF inhibitor to a patient in need of cancer treatment generally, and mUC treatment more specifically, wherein the patient harbors at least one FGFR2 genetic alteration or FGFR3 genetic alteration and at least one BRAF genetic alteration.

Also described herein are methods of treating cancer generally, and mUC more specifically, in a patient comprising, consisting of, or consisting essential of: (a) evaluating a biological sample from the patient for the presence of at least one FGFR2 genetic alteration or FGFR3 genetic alteration and at least one BRAF genetic alteration; and (b) treating the patient with an FGFR inhibitor in combination with a BRAF inhibitor if the at least one FGFR2 genetic alteration or FGFR3 genetic alteration and the at least one BRAF genetic alteration are present in the sample.

Still further described herein are an FGFR inhibitor and a BRAF inhibitor for use in the treatment of cancer generally, and mUC more specifically, in a patient who harbors at least one FGFR2 genetic alteration or FGFR3 genetic alteration and at least one BRAF genetic alteration. Also described herein is an FGFR inhibitor for use in the treatment of cancer generally, and mUC more specifically, in a patient who harbors at least one FGFR2 genetic alteration or FGFR3 genetic alteration and at least one BRAF genetic alteration, wherein the FGFR inhibitor is to be used in combination with a BRAF inhibitor. Also described herein is a BRAF inhibitor for use in the treatment of cancer generally, and mUC more specifically, in a patient who harbors at least one FGFR2 genetic alteration or FGFR3 genetic alteration and at least one BRAF genetic alteration, wherein the BRAF inhibitor is to be used in combination with an FGFR inhibitor.

Also described herein are uses of an FGFR inhibitor for the manufacture of a medicament for the treatment of cancer generally, and mUC more specifically, in a patient who harbors at least one FGFR2 genetic alteration or FGFR3 genetic alteration and at least one BRAF genetic alteration, wherein the FGFR inhibitor is to be used in combination with a BRAF inhibitor. Also described herein are uses of a BRAF inhibitor for the manufacture of a medicament for the treatment of cancer generally, and mUC more specifically, in a patient who harbors at least one FGFR2 genetic alteration or FGFR3 genetic alteration and at least one BRAF genetic alteration, wherein the BRAF inhibitor is to be used in combination with an FGFR inhibitor.

In certain embodiments, administration of the FGFR inhibitor in combination with a BRAF inhibitor provides improved anti-tumor activity, as measured by OS or PFS, relative to a patient or patient population with cancer generally, and mUC more specifically, who was not receiving treatment with an FGFR inhibitor in combination with a BRAF inhibitor. In certain embodiments, administration of an FGFR inhibitor in combination with a BRAF inhibitor provides improved anti-tumor activity, as measured by OS, relative to a patient or patient population with cancer generally, and mUC more specifically, who was not receiving treatment with an FGFR inhibitor in combination with a BRAF inhibitor. In certain embodiments, administration of an FGFR inhibitor in combination with a BRAF inhibitor provides improved anti-tumor activity, as measured by PFS, relative to a patient or patient population with cancer generally, and mUC more specifically, who was not receiving treatment with an FGFR inhibitor in combination with a BRAF inhibitor.

In certain embodiments, the improvement in anti-tumor activity is relative to treatment with an FGFR inhibitor. In certain embodiments, the improvement in anti-tumor activity is relative to treatment with placebo. In certain embodiments, the improvement in anti-tumor activity is relative to no treatment. In certain embodiments, the improvement in anti-tumor activity is relative to standard of care. In certain embodiments, the improvement in anti-tumor activity is relative to a patient population without cancer generally, and mUC more specifically.

Further described herein are methods of predicting duration of PFS in a patient, in particular a human patient, having cancer generally, and mUC more specifically, in particular in a patient on treatment with an FGFR inhibitor, the method comprising evaluating a biological sample from the patient for the presence of at least one FGFR2 genetic alteration or FGFR3 genetic alteration and at least one BRAF genetic alteration, wherein the presence of at least one FGFR2 genetic alteration or FGFR3 genetic alteration and at least one BRAF genetic alteration indicates a shorter duration of PFS, relative to a patient, in particular a human patient, having cancer generally, and mUC more specifically, who does not harbor at least one BRAF genetic alteration, or relative to a patient, in particular a human patient, having cancer generally, and mUC more specifically, who does not harbor at least one FGFR2 genetic alteration or FGFR3 genetic alteration and at least one BRAF genetic alteration.

Still further described herein are methods of predicting duration of OS in a patient, in particular a human patient, having cancer generally, and mUC more specifically, in particular in a patient on treatment with an FGFR inhibitor, the method comprising evaluating a biological sample from the patient for the presence of at least one FGFR2 genetic alteration or FGFR3 genetic alteration and at least one BRAF genetic alteration, wherein the presence of at least one FGFR2 genetic alteration or FGFR3 genetic alteration and at least one BRAF genetic alteration indicates a shorter duration of OS relative to a patient, in particular a human patient, having cancer generally, and mUC more specifically, who does not harbor at least one BRAF genetic alteration, or relative to a patient, in particular a human patient, having cancer generally, and mUC more specifically, who does not harbor at least one FGFR2 genetic alteration or FGFR3 genetic alteration and at least one BRAF genetic alteration.

Also described herein are methods of improving OS in a patient with cancer generally, and mUC more specifically, relative to a patient with cancer generally, and mUC more specifically, who was not receiving treatment with an FGFR inhibitor in combination with a BRAF inhibitor, said method comprising providing to said patient an FGFR inhibitor in combination with a BRAF inhibitor, wherein said patient harbors at least one FGFR2 genetic alteration or FGFR3 genetic alteration and at least one BRAF genetic alteration.

Also described herein is an FGFR inhibitor for use in improving OS in a patient with cancer generally, and mUC more specifically, relative to a patient with cancer generally, and mUC more specifically, who was not receiving treatment with an FGFR inhibitor in combination with a BRAF inhibitor, wherein the FGFR inhibitor is to be used in combination with a BRAF inhibitor and wherein said patient harbors at least one FGFR2 genetic alteration or FGFR3 genetic alteration and at least one BRAF genetic alteration. Also described herein is a BRAF inhibitor for use in improving OS in a patient with cancer generally, and mUC more specifically, relative to a patient with cancer generally, and mUC more specifically, who was not receiving treatment with a BRAF inhibitor in combination with an FGFR inhibitor, wherein the BRAF inhibitor is to be used in combination with an FGFR inhibitor and wherein said patient harbors at least one FGFR2 genetic alteration or FGFR3 genetic alteration and at least one BRAF genetic alteration.

Further described herein are methods of improving PFS in a patient with cancer generally, and mUC more specifically, relative to a patient with cancer generally, and mUC more specifically, who was not receiving treatment with an FGFR inhibitor in combination with a BRAF inhibitor, said method comprising providing to said patient an FGFR inhibitor in combination with a BRAF inhibitor, wherein said patient harbors at least one FGFR2 genetic alteration or FGFR3 genetic alteration and at least one BRAF genetic alteration.

Also described herein is an FGFR inhibitor for use in improving PFS in a patient with cancer generally, and mUC more specifically, relative to a patient with cancer generally, and mUC more specifically, who was not receiving treatment with an FGFR inhibitor in combination with a BRAF inhibitor, wherein the FGFR inhibitor is to be used in combination with a BRAF inhibitor and wherein said patient harbors at least one FGFR2 genetic alteration or FGFR3 genetic alteration and at least one BRAF genetic alteration. Also described herein is a BRAF inhibitor for use in improving PFS in a patient with cancer generally, and mUC more specifically, relative to a patient with cancer generally, and mUC more specifically, who was not receiving treatment with a BRAF inhibitor in combination with an FGFR inhibitor, wherein the BRAF inhibitor is to be used in combination with an FGFR inhibitor and wherein said patient harbors at least one FGFR2 genetic alteration or FGFR3 genetic alteration and at least one BRAF genetic alteration.

Said methods and uses also encompass administration of at least one, two, three or four FGFR in combination with at least one, two, three or four BRAF inhibitors to a patient that has been diagnosed with cancer generally, and mUC more specifically. In addition to administration of FGFR and BRAF inhibitors, as described herein, said methods and uses encompass administration of at least one, two three, or four EGFR, CCND1, ARID1A, ErbB2, or TERT inhibitors, or any combination thereof, to a patient who harbors at least one EGFR, CCND1, ARID1A, ErbB2, or TERT genetic alterations, or any combination thereof, respectively. In certain embodiments, in addition to administration of FGFR and BRAF inhibitors, as described herein, said methods and uses also encompass administration of at least one, two, three, or four EGFR inhibitors if the patient also harbors at least one EGFR genetic alteration. In certain embodiments, in addition to administration of FGFR and BRAF inhibitors, as described herein, said methods and uses also encompass administration of at least one, two, three, or four CCND1 inhibitors if the patient also harbors at least one CCND1 genetic alteration. In certain embodiments, in addition to administration of FGFR and BRAF inhibitors, as described herein, said methods and uses also encompass administration of at least one, two, three, or four ARID1A inhibitors if the patient also harbors at least one ARID1A genetic alteration. In certain embodiments, in addition to administration of FGFR and BRAF inhibitors, as described herein, said methods and uses also encompass administration of at least one, two, three, or four ErbB2 inhibitors if the patient also harbors at least one ErbB2 genetic alteration. In certain embodiments, in addition to administration of FGFR and BRAF inhibitors, as described herein, said methods and uses also encompass administration of at least one, two, three, or four TERT inhibitors if the patient also harbors at least one TERT genetic alteration.

In certain embodiments, in addition to administration of FGFR and BRAF inhibitors, as described herein, said methods and uses also encompass administration of at least one, two, three, or four EGFR inhibitors if the patient also harbors at least one EGFR genetic alteration and at least one, two, three or four CCND1 inhibitors if the patient also harbors at least one CCND1 genetic alteration.

FGFR and ARID1A Combination Therapies

Described herein are methods of treating cancer generally, and mUC more specifically, comprising, consisting of, or consisting essential of, administering an FGFR inhibitor in combination with an ARID1A inhibitor to a patient in need of cancer treatment generally, and mUC treatment more specifically, wherein the patient harbors at least one FGFR2 genetic alteration or FGFR3 genetic alteration and at least one ARID1A genetic alteration.

Also described herein are methods of treating cancer generally, and mUC more specifically, in a patient comprising, consisting of, or consisting essential of: (a) evaluating a biological sample from the patient for the presence of at least one FGFR2 genetic alteration or FGFR3 genetic alteration and at least one ARID1A genetic alteration; and (b) treating the patient with an FGFR inhibitor in combination with an ARID1A inhibitor if the at least one FGFR2 genetic alteration or FGFR3 genetic alteration and the at least one ARID1A genetic alteration are present in the sample.

Still further described herein are an FGFR inhibitor and an ARID1A inhibitors for use in the treatment of cancer generally, and mUC more specifically, in a patient who harbors at least one FGFR2 genetic alteration or FGFR3 genetic alteration and at least one ARID1A genetic alteration. Also described herein is an FGFR inhibitor for use in the treatment of cancer generally, and mUC more specifically, in a patient who harbors at least one FGFR2 genetic alteration or FGFR3 genetic alteration and at least one ARID1A genetic alteration wherein the FGFR inhibitor is to be used in combination with an ARID1A inhibitor. Also described herein is an ARID1A inhibitor for use in the treatment of cancer generally, and mUC more specifically, in a patient who harbors at least one FGFR2 genetic alteration or FGFR3 genetic alteration and at least one ARID1A genetic alteration wherein the ARID1A inhibitor is to be used in combination with an FGFR inhibitor.

Also described herein are uses of an FGFR inhibitor for the manufacture of a medicament for the treatment of cancer generally, and mUC more specifically, in a patient who harbors at least one FGFR2 genetic alteration or FGFR3 genetic alteration and at least one ARID1A genetic alteration, wherein the FGFR inhibitor is to be used in combination with an ARID1A inhibitor. Also described herein are uses of an ARID1A inhibitor for the manufacture of a medicament for the treatment of cancer generally, and mUC more specifically, in a patient who harbors at least one FGFR2 genetic alteration or FGFR3 genetic alteration and at least one ARID1A genetic alteration, wherein the ARID1A inhibitor is to be used in combination with an FGFR inhibitor.

In certain embodiments, administration of the FGFR inhibitor in combination with an ARID1A inhibitor provides improved anti-tumor activity, as measured by OS or PFS, relative to a patient or patient population with cancer generally, and mUC more specifically, who was not receiving treatment with an FGFR inhibitor in combination with an ARID1A inhibitor. In certain embodiments, administration of the FGFR inhibitor in combination with an ARID1A inhibitor provides improved anti-tumor activity, as measured by OS, relative to a patient or patient population with cancer generally, and mUC more specifically, who was not receiving treatment with an FGFR inhibitor in combination with an ARID1A inhibitor. In certain embodiments, administration of the FGFR inhibitor in combination with an ARID1A inhibitor provides improved anti-tumor activity, as measured by PFS, relative to a patient or patient population with cancer generally, and mUC more specifically, who was not receiving treatment with an FGFR inhibitor in combination with an ARID1A inhibitor.

In certain embodiments, the improvement in anti-tumor activity is relative to treatment with an FGFR inhibitor. In certain embodiments, the improvement in anti-tumor activity is relative to treatment with placebo. In certain embodiments, the improvement in anti-tumor activity is relative to no treatment. In certain embodiments, the improvement in anti-tumor activity is relative to standard of care. In certain embodiments, the improvement in anti-tumor activity is relative to a patient population without cancer generally, and mUC more specifically.

Further described herein are methods of predicting duration of PFS in a patient, in particular a human patient, having cancer generally, and mUC more specifically, in particular in a patient on treatment with an FGFR inhibitor, the method comprising evaluating a biological sample from the patient for the presence of at least one FGFR2 genetic alteration or FGFR3 genetic alteration and at least one ARID1A genetic alteration, wherein the presence of at least one FGFR2 genetic alteration or FGFR3 genetic alteration and at least one ARID1A genetic alteration indicates a shorter duration of PFS, relative to a patient, in particular a human patient, having cancer generally, and mUC more specifically, who does not harbor at least one ARID1A genetic alteration, or relative to a patient, in particular a human patient, having cancer generally, and mUC more specifically, who does not harbor at least one FGFR2 genetic alteration or FGFR3 genetic alteration and at least one ARID1A genetic alteration.

Still further described herein are methods of predicting duration of OS in a patient, in particular a human patient, having cancer generally, and mUC more specifically, the method comprising evaluating a biological sample from the patient for the presence of at least one FGFR2 genetic alteration or FGFR3 genetic alteration and at least one ARID1A genetic alteration, wherein the presence of at least one FGFR2 genetic alteration or FGFR3 genetic alteration and at least one ARID1A genetic alteration indicates a shorter duration of OS relative to a patient, in particular a human patient, having cancer generally, and mUC more specifically, who does not harbor at least one ARID1A genetic alteration, or relative to a patient, in particular a human patient, having cancer generally, and mUC more specifically, who does not harbor at least one FGFR2 genetic alteration or FGFR3 genetic alteration and at least one ARID1A genetic alteration.

Also described herein are methods of improving OS in a patient with cancer generally, and mUC more specifically, relative to a patient with cancer generally, and mUC more specifically, who was not receiving treatment with an FGFR inhibitor in combination with an ARID1A inhibitor, said method comprising providing to said patient an FGFR inhibitor in combination with an ARID1A inhibitor, wherein said patient harbors at least one FGFR2 genetic alteration or FGFR3 genetic alteration and at least one ARID1A genetic alteration.

Also described herein is an FGFR inhibitor for use in improving OS in a patient with cancer generally, and mUC more specifically, relative to a patient with cancer generally, and mUC more specifically, who was not receiving treatment with an FGFR inhibitor in combination with an ARID1A inhibitor, wherein the FGFR inhibitor is to be used in combination with an ARID1A inhibitor and wherein said patient harbors at least one FGFR2 genetic alteration or FGFR3 genetic alteration and at least one ARID1A genetic alteration. Also described herein is an ARID1A inhibitor for use in improving OS in a patient with cancer generally, and mUC more specifically, relative to a patient with cancer generally, and mUC more specifically, who was not receiving treatment with an ARID1A inhibitor in combination with an FGFR inhibitor, wherein the ARID1A inhibitor is to be used in combination with an FGFR inhibitor and wherein said patient harbors at least one FGFR2 genetic alteration or FGFR3 genetic alteration and at least one ARID1A genetic alteration.

Further described herein are methods of improving PFS in a patient with cancer generally, and mUC more specifically, relative to a patient with cancer generally, and mUC more specifically, who was not receiving treatment with an FGFR inhibitor in combination with an ARID1A inhibitor, said method comprising providing to said patient an FGFR inhibitor in combination with an ARID1A inhibitor, wherein said patient harbors at least one FGFR2 genetic alteration or FGFR3 genetic alteration and at least one ARID1A genetic alteration.

Also described herein is an FGFR inhibitor for use in improving PFS in a patient with cancer generally, and mUC more specifically, relative to a patient with cancer generally, and mUC more specifically, who was not receiving treatment with an FGFR inhibitor in combination with an ARID1A inhibitor, wherein the FGFR inhibitor is to be used in combination with an ARID1A inhibitor and wherein said patient harbors at least one FGFR2 genetic alteration or FGFR3 genetic alteration and at least one ARID1A genetic alteration. Also described herein is an ARID1A inhibitor for use in improving PFS in a patient with cancer generally, and mUC more specifically, relative to a patient with cancer generally, and mUC more specifically, who was not receiving treatment with an ARID1A inhibitor in combination with an FGFR inhibitor, wherein the ARID1A inhibitor is to be used in combination with an FGFR inhibitor and wherein said patient harbors at least one FGFR2 genetic alteration or FGFR3 genetic alteration and at least one ARID1A genetic alteration.

Said methods and uses also encompass administration of at least one, two, three or four FGFR in combination with at least one, two, three or four ARID1A inhibitors to a patient that has been diagnosed with cancer generally, and mUC more specifically. In addition to administration of FGFR and ARID1A inhibitors, as described herein, said methods and uses encompass administration of at least one, two three, or four EGFR, CCND1, BRAF, ErbB2, or TERT inhibitors, or any combination thereof, to a patient who harbors at least one EGFR, CCND1, BRAF, ErbB2, or TERT genetic alterations, or any combination thereof, respectively. In certain embodiments, in addition to administration of FGFR and ARID1A inhibitors, as described herein, said methods and uses also encompass administration of at least one, two, three, or four EGFR inhibitors if the patient also harbors at least one EGFR genetic alteration. In certain embodiments, in addition to administration of FGFR and ARID1A inhibitors, as described herein, said methods and uses also encompass administration of at least one, two, three, or four CCND1 inhibitors if the patient also harbors at least one CCND1 genetic alteration. In certain embodiments, in addition to administration of FGFR and ARID1A inhibitors, as described herein, said methods and uses also encompass administration of at least one, two, three, or four BRAF inhibitors if the patient also harbors at least one BRAF genetic alteration. In certain embodiments, in addition to administration of FGFR and ARID1A inhibitors, as described herein, said methods and uses also encompass administration of at least one, two, three, or four ErbB2 inhibitors if the patient also harbors at least one ErbB2 genetic alteration. In certain embodiments, in addition to administration of FGFR and ARID1A inhibitors, as described herein, said methods and uses also encompass administration of at least one, two, three, or four TERT inhibitors if the patient also harbors at least one TERT genetic alteration.

FGFR and ErbB2 Combination Therapies

Described herein are methods of treating cancer generally, and mUC more specifically, comprising, consisting of, or consisting essential of, administering an FGFR inhibitor in combination with an ErbB2 inhibitor to a patient in need of cancer treatment generally, and mUC treatment more specifically, wherein the patient harbors at least one FGFR2 genetic alteration or FGFR3 genetic alteration and at least one ErbB2 genetic alteration.

Also described herein are methods of treating cancer generally, and mUC more specifically, in a patient comprising, consisting of, or consisting essential of: (a) evaluating a biological sample from the patient for the presence of at least one FGFR2 genetic alteration or FGFR3 genetic alteration and at least one ERBB2 genetic alteration; and (b) treating the patient with an FGFR inhibitor in combination with an ErbB2 inhibitor if the at least one FGFR2 genetic alteration or FGFR3 genetic alteration and the at least one ErbB2 genetic alteration are present in the sample.

Still further described herein are an FGFR inhibitor and an ErbB2 inhibitor for use in the treatment of cancer generally, and mUC more specifically, in a patient who harbors at least one FGFR2 genetic alteration or FGFR3 genetic alteration and at least one ErbB2 genetic alteration. Also described herein is an FGFR inhibitor for use in the treatment of cancer generally, and mUC more specifically, in a patient who harbors at least one FGFR2 genetic alteration or FGFR3 genetic alteration and at least one ErbB2 genetic alteration, wherein the FGFR inhibitor is to be used in combination with an ErbB2 inhibitor. Also described herein is an ErbB2 inhibitor for use in the treatment of cancer generally, and mUC more specifically, in a patient who harbors at least one FGFR2 genetic alteration or FGFR3 genetic alteration and at least one ErbB2 genetic alteration, wherein the ErbB2 inhibitor is to be used in combination with an FGFR inhibitor.

Also described herein are uses of an FGFR inhibitor for the manufacture of a medicament for the treatment of cancer generally, and mUC more specifically, in a patient who harbors at least one FGFR2 genetic alteration or FGFR3 genetic alteration and at least one ErbB2 genetic alteration, wherein the FGFR inhibitor is to be used in combination with an ErbB2 inhibitor. Also described herein are uses of an ErbB2 inhibitor for the manufacture of a medicament for the treatment of cancer generally, and mUC more specifically, in a patient who harbors at least one FGFR2 genetic alteration or FGFR3 genetic alteration and at least one ErbB2 genetic alteration, wherein the ErbB2 inhibitor is to be used in combination with an FGFR inhibitor.

In certain embodiments, administration of the FGFR inhibitor in combination with an ErbB2 inhibitor provides improved anti-tumor activity, as measured by OS or PFS, relative to a patient or patient population with cancer generally, and mUC more specifically, who was not receiving treatment with an FGFR inhibitor in combination with an ErbB2 inhibitor. In certain embodiments, administration of the FGFR inhibitor in combination with an ErbB2 inhibitor provides improved anti-tumor activity, as measured by OS, relative to a patient or patient population with cancer generally, and mUC more specifically, who was not receiving treatment with an FGFR inhibitor in combination with an ErbB2 inhibitor. In certain embodiments, administration of the FGFR inhibitor in combination with an ErbB2 inhibitor provides improved anti-tumor activity, as measured by PFS, relative to a patient or patient population with cancer generally, and mUC more specifically, who was not receiving treatment with an FGFR inhibitor in combination with an ErbB2 inhibitor.

In certain embodiments, the improvement in anti-tumor activity is relative to treatment with an FGFR inhibitor. In certain embodiments, the improvement in anti-tumor activity is relative to treatment with placebo. In certain embodiments, the improvement in anti-tumor activity is relative to no treatment. In certain embodiments, the improvement in anti-tumor activity is relative to standard of care. In certain embodiments, the improvement in anti-tumor activity is relative to a patient population without cancer generally, and mUC more specifically.

Further described herein are methods of predicting duration of PFS in a patient, in particular a human patient, having cancer generally, and mUC more specifically, in particular a patient on treatment with an FGFR inhibitor, the method comprising evaluating a biological sample from the patient for the presence of at least one FGFR2 genetic alteration or FGFR3 genetic alteration and at least one ErbB2 genetic alteration, wherein the presence of at least one FGFR2 genetic alteration or FGFR3 genetic alteration and at least one ErbB2 genetic alteration indicates a shorter duration of PFS, relative to a patient, in particular a human patient, having cancer generally, and mUC more specifically, who does not harbor at least one ErbB2 genetic alteration, or relative to a patient, in particular a human patient, having cancer generally, and mUC more specifically, who does not harbor at least one FGFR2 genetic alteration or FGFR3 genetic alteration and at least one ErbB2 genetic alteration.

Still further described herein are methods of predicting duration of OS in a patient, in particular a human patient, having cancer generally, and mUC more specifically, in particular in a patient on treatment with an FGFR inhibitor. the method comprising evaluating a biological sample from the patient for the presence of at least one FGFR2 genetic alteration or FGFR3 genetic alteration and at least one ErbB2 genetic alteration, wherein the presence of at least one FGFR2 genetic alteration or FGFR3 genetic alteration and at least one ErbB2 genetic alteration indicates a shorter duration of OS relative to a patient, in particular a human patient, having cancer generally, and mUC more specifically, who does not harbor at least one ErbB2 genetic alteration, or relative to a patient, in particular a human patient, having cancer generally, and mUC more specifically, who does not harbor at least one FGFR2 genetic alteration or FGFR3 genetic alteration and at least one ErbB2 genetic alteration.

Also described herein are methods of improving OS in a patient with cancer generally, and mUC more specifically, relative to a patient with cancer generally, and mUC more specifically, who was not receiving treatment with an FGFR inhibitor in combination with an ErbB2 inhibitor, said method comprising providing to said patient an FGFR inhibitor in combination with an ErbB2 inhibitor, wherein said patient harbors at least one FGFR2 genetic alteration or FGFR3 genetic alteration and at least one ErbB2 genetic alteration.

Also described herein is an FGFR inhibitor for use in improving OS in a patient with cancer generally, and mUC more specifically, relative to a patient with cancer generally, and mUC more specifically, who was not receiving treatment with an FGFR inhibitor in combination with an ErbB2 inhibitor, wherein the FGFR inhibitor is to be used in combination with an ErbB2 inhibitor and wherein said patient harbors at least one FGFR2 genetic alteration or FGFR3 genetic alteration and at least one ErbB2 genetic alteration. Also described herein is an ErbB2 inhibitor for use in improving OS in a patient with cancer generally, and mUC more specifically, relative to a patient with cancer generally, and mUC more specifically, who was not receiving treatment with an ErbB2 inhibitor in combination with an FGFR inhibitor, wherein the ErbB2 inhibitor is to be used in combination with an FGFR inhibitor and wherein said patient harbors at least one FGFR2 genetic alteration or FGFR3 genetic alteration and at least one ErbB2 genetic alteration.

Further described herein are methods of improving PFS in a patient with cancer generally, and mUC more specifically, relative to a patient with cancer generally, and mUC more specifically, who was not receiving treatment with an FGFR inhibitor in combination with an ErbB2 inhibitor, said method comprising providing to said patient an FGFR inhibitor in combination with an ErbB2 inhibitor, wherein said patient harbors at least one FGFR2 genetic alteration or FGFR3 genetic alteration and at least one ErbB2 genetic alteration.

Also described herein is an FGFR inhibitor for use in improving PFS in a patient with cancer generally, and mUC more specifically, relative to a patient with cancer generally, and mUC more specifically, who was not receiving treatment with an FGFR inhibitor in combination with an ErbB2 inhibitor, wherein the FGFR inhibitor is to be used in combination with an ErbB2 inhibitor and wherein said patient harbors at least one FGFR2 genetic alteration or FGFR3 genetic alteration and at least one ErbB2 genetic alteration. Also described herein is an ErbB2 inhibitor for use in improving PFS in a patient with cancer generally, and mUC more specifically, relative to a patient with cancer generally, and mUC more specifically, who was not receiving treatment with an ErbB2 inhibitor in combination with an FGFR inhibitor, wherein the ErbB2 inhibitor is to be used in combination with an FGFR inhibitor and wherein said patient harbors at least one FGFR2 genetic alteration or FGFR3 genetic alteration and at least one ErbB2 genetic alteration.

Said methods and uses also encompass administration of at least one, two, three or four FGFR in combination with at least one, two, three or four ErbB2 inhibitors to a patient that has been diagnosed with cancer generally, and mUC more specifically. In addition to administration of FGFR and ErbB2 inhibitors, as described herein, said methods and uses encompass administration of at least one, two three, or four EGFR, CCND1, BRAF, ARID1A, or TERT inhibitors, or any combination thereof, to a patient who harbors at least one EGFR, CCND1, BRAF, ARID1A, or TERT genetic alterations, or any combination thereof, respectively. In certain embodiments, in addition to administration of FGFR and ERBB2 inhibitors, as described herein, said methods and uses also encompass administration of at least one, two, three, or four EGFR inhibitors if the patient also harbors at least one EGFR genetic alteration. In certain embodiments, in addition to administration of FGFR and ErbB2 inhibitors, as described herein, said methods and uses also encompass administration of at least one, two, three, or four CCND1 inhibitors if the patient also harbors at least one CCND1 genetic alteration. In certain embodiments, in addition to administration of FGFR and ErbB2 inhibitors, as described herein, said methods and uses also encompass administration of at least one, two, three, or four BRAF inhibitors if the patient also harbors at least one BRAF genetic alteration. In certain embodiments, in addition to administration of FGFR and ErbB2 inhibitors, as described herein, said methods and uses also encompass administration of at least one, two, three, or four ARID1A inhibitors if the patient also harbors at least one ARID1A genetic alteration. In certain embodiments, in addition to administration of FGFR and ErbB2 inhibitors, as described herein, said methods and uses also encompass administration of at least one, two, three, or four TERT inhibitors if the patient also harbors at least one TERT genetic alteration.

FGFR and TERT Combination Therapies

Described herein are methods of treating cancer generally, and mUC more specifically, comprising, consisting of, or consisting essential of, administering an FGFR inhibitor in combination with a TERT inhibitor to a patient in need of cancer treatment generally, and mUC treatment more specifically, wherein the patient harbors at least one FGFR2 genetic alteration or FGFR3 genetic alteration and at least one TERT genetic alteration.

Also described herein are methods of treating cancer generally, and mUC more specifically, in a patient comprising, consisting of, or consisting essential of: (a) evaluating a biological sample from the patient for the presence of at least one FGFR2 genetic alteration or FGFR3 genetic alteration and at least one TERT genetic alteration; and (b) treating the patient with an FGFR inhibitor in combination with a TERT inhibitor if the at least one FGFR2 genetic alteration or FGFR3 genetic alteration and the at least one TERT genetic alteration are present in the sample.

Still further described herein are an FGFR inhibitor and a TERT inhibitors for use in the treatment of cancer generally, and mUC more specifically, in a patient who harbors at least one FGFR2 genetic alteration or FGFR3 genetic alteration and at least one TERT genetic alteration. Also described herein is an FGFR inhibitor for use in the treatment of cancer generally, and mUC more specifically, in a patient who harbors at least one FGFR2 genetic alteration or FGFR3 genetic alteration and at least one TERT genetic alteration, wherein the FGFR inhibitor is to be used in combination with a TERT inhibitor. Also described herein is a TERT inhibitor for use in the treatment of cancer generally, and mUC more specifically, in a patient who harbors at least one FGFR2 genetic alteration or FGFR3 genetic alteration and at least one TERT genetic alteration, wherein the TERT inhibitor is to be used in combination with an FGFR inhibitor.

Also described herein are uses of an FGFR inhibitor for the manufacture of a medicament for the treatment of cancer generally, and mUC more specifically, in a patient who harbors at least one FGFR2 genetic alteration or FGFR3 genetic alteration and at least one TERT genetic alteration, wherein the FGFR inhibitor is to be used in combination with a TERT inhibitor. Also described herein are uses of a TERT inhibitor for the manufacture of a medicament for the treatment of cancer generally, and mUC more specifically, in a patient who harbors at least one FGFR2 genetic alteration or FGFR3 genetic alteration and at least one TERT genetic alteration, wherein the TERT inhibitor is to be used in combination with an FGFR inhibitor.

In certain embodiments, administration of the FGFR inhibitor in combination with a TERT inhibitor provides improved anti-tumor activity, as measured by OS or PFS, relative to a patient or patient population with cancer generally, and mUC more specifically, who was not receiving treatment with an FGFR inhibitor in combination with a TERT inhibitor. In certain embodiments, administration of the FGFR inhibitor in combination with a TERT inhibitor provides improved anti-tumor activity, as measured by OS, relative to a patient or patient population with cancer generally, and mUC more specifically, who was not receiving treatment with an FGFR inhibitor in combination with a TERT inhibitor. In certain embodiments, administration of the FGFR inhibitor in combination with a TERT inhibitor provides improved anti-tumor activity, as measured by PFS, relative to a patient or patient population with cancer generally, and mUC more specifically, who was not receiving treatment with an FGFR inhibitor in combination with a TERT inhibitor.

In certain embodiments, the improvement in anti-tumor activity is relative to treatment with an FGFR inhibitor. In certain embodiments, the improvement in anti-tumor activity is relative to treatment with placebo. In certain embodiments, the improvement in anti-tumor activity is relative to no treatment. In certain embodiments, the improvement in anti-tumor activity is relative to standard of care. In certain embodiments, the improvement in anti-tumor activity is relative to a patient population without cancer generally, and mUC more specifically.

Further described herein are methods of predicting duration of PFS in a patient, in particular a human patient, having cancer generally, and mUC more specifically, in particular in a patient on treatment with an FGFR inhibitor, the method comprising evaluating a biological sample from the patient for the presence of at least one FGFR2 genetic alteration or FGFR3 genetic alteration and at least one TERT genetic alteration, wherein the presence of at least one FGFR2 genetic alteration or FGFR3 genetic alteration and at least one TERT genetic alteration indicates a shorter duration of PFS, relative to a patient, in particular a human patient, having cancer generally, and mUC more specifically, who does not harbor at least one TERT genetic alteration, or relative to a patient, in particular a human patient, having cancer generally, and mUC more specifically, who does not harbor at least one FGFR2 genetic alteration or FGFR3 genetic alteration and at least one TERT genetic alteration.

Still further described herein are methods of predicting duration of OS in a patient, in particular a human patient, having cancer generally, and mUC more specifically, in particular in a patient on treatment with an FGFR inhibitor, the method comprising evaluating a biological sample from the patient for the presence of at least one FGFR2 genetic alteration or FGFR3 genetic alteration and at least one TERT genetic alteration, wherein the presence of at least one FGFR2 genetic alteration or FGFR3 genetic alteration and at least one TERT genetic alteration indicates a shorter duration of OS relative to a patient, in particular a human patient, having cancer generally, and mUC more specifically, who does not harbor at least one TERT genetic alteration, or relative to a patient, in particular a human patient, having cancer generally, and mUC more specifically, who does not harbor at least one FGFR2 genetic alteration or FGFR3 genetic alteration and at least one TERT genetic alteration.

Also described herein are methods of improving OS in a patient with cancer generally, and mUC more specifically, relative to a patient with cancer generally, and mUC more specifically, who was not receiving treatment with an FGFR inhibitor in combination with a TERT inhibitor, said method comprising providing to said patient an FGFR inhibitor in combination with a TERT inhibitor, wherein said patient harbors at least one FGFR2 genetic alteration or FGFR3 genetic alteration and at least one TERT genetic alteration.

Also described herein is an FGFR inhibitor for use in improving OS in a patient with cancer generally, and mUC more specifically, relative to a patient with cancer generally, and mUC more specifically, who was not receiving treatment with an FGFR inhibitor in combination with a TERT inhibitor, wherein the FGFR inhibitor is to be used in combination with a TERT inhibitor and wherein said patient harbors at least one FGFR2 genetic alteration or FGFR3 genetic alteration and at least one TERT genetic alteration. Also described herein is a TERT inhibitor for use in improving OS in a patient with cancer generally, and mUC more specifically, relative to a patient with cancer generally, and mUC more specifically, who was not receiving treatment with a TERT inhibitor in combination with an FGFR inhibitor, wherein the TERT inhibitor is to be used in combination with an FGFR inhibitor and wherein said patient harbors at least one FGFR2 genetic alteration or FGFR3 genetic alteration and at least one TERT genetic alteration.

Further described herein are methods of improving PFS in a patient with cancer generally, and mUC more specifically, relative to a patient with cancer generally, and mUC more specifically, who was not receiving treatment with an FGFR inhibitor in combination with a TERT inhibitor, said method comprising providing to said patient an FGFR inhibitor in combination with a TERT inhibitor, wherein said patient harbors at least one FGFR2 genetic alteration or FGFR3 genetic alteration and at least one TERT genetic alteration.

Also described herein is an FGFR inhibitor for use in improving PFS in a patient with cancer generally, and mUC more specifically, relative to a patient with cancer generally, and mUC more specifically, who was not receiving treatment with an FGFR inhibitor in combination with a TERT inhibitor, wherein the FGFR inhibitor is to be used in combination with a TERT inhibitor and wherein said patient harbors at least one FGFR2 genetic alteration or FGFR3 genetic alteration and at least one TERT genetic alteration. Also described herein is a TERT inhibitor for use in improving PFS in a patient with cancer generally, and mUC more specifically, relative to a patient with cancer generally, and mUC more specifically, who was not receiving treatment with a TERT inhibitor in combination with an FGFR inhibitor, wherein the TERT inhibitor is to be used in combination with an FGFR inhibitor and wherein said patient harbors at least one FGFR2 genetic alteration or FGFR3 genetic alteration and at least one TERT genetic alteration.

Said methods and uses also encompass administration of at least one, two, three or four FGFR in combination with at least one, two, three or four TERT inhibitors to a patient that has been diagnosed with cancer generally, and mUC more specifically. In addition to administration of FGFR and TERT inhibitors, as described herein, said methods and uses encompass administration of at least one, two three, or four EGFR, BRAF, ARID1A, ErbB2, or CCND1 inhibitors, or any combination thereof, to a patient who harbors at least one EGFR, BRAF, ARID1A, ErbB2, or CCND1 genetic alterations, or any combination thereof, respectively. In certain embodiments, in addition to administration of FGFR and TERT inhibitors, as described herein, said methods and uses also encompass administration of at least one, two, three, or four EGFR inhibitors if the patient also harbors at least one EGFR genetic alteration. In certain embodiments, in addition to administration of FGFR and TERT inhibitors, as described herein, said methods and uses also encompass administration of at least one, two, three, or four CCND1 inhibitors if the patient also harbors at least one CCND1 genetic alteration. In certain embodiments, in addition to administration of FGFR and TERT inhibitors, as described herein, said methods and uses also encompass administration of at least one, two, three, or four BRAF inhibitors if the patient also harbors at least one BRAF genetic alteration. In certain embodiments, in addition to administration of FGFR and TERT inhibitors, as described herein, said methods and uses also encompass administration of at least one, two, three, or four ARID1A inhibitors if the patient also harbors at least one ARID1A genetic alteration. In certain embodiments, in addition to administration of FGFR and TERT inhibitors, as described herein, said methods and uses also encompass administration of at least one, two, three, or four ErbB2 inhibitors if the patient also harbors at least one ErbB2 genetic alteration.

### Evaluating a Sample for the Presence of Genetic Alterations

Also described herein are methods of treating cancer generally, and mUC more specifically, in a patient comprising, consisting of, or consisting essential of: (a) evaluating a biological sample from the patient for the presence of at least one FGFR2 genetic alteration or FGFR3 genetic alteration and at least one BRAF, EGFR, CCND1, ARID1A, ErbB2 or TERT genetic alteration; and (b) treating the patient with an FGFR inhibitor in combination with a BRAF, EGFR, CCND1, ARID1A, ErbB2 or TERT inhibitor if the at least one FGFR2 genetic alteration or FGFR3 genetic alteration and the at least one BRAF, EGFR, CCND1, ARID1A, ErbB2 or TERT genetic alteration are present in the sample, respectively.

Also described herein are methods of treating cancer generally, and mUC more specifically, in a patient comprising, consisting of, or consisting essential of: (a) evaluating a biological sample from the patient for the presence of at least one FGFR2 genetic alteration or FGFR3 genetic alteration and at least one FGFR1 genetic alteration; and (b) treating the patient with an FGFR inhibitor in combination with a second FGFR inhibitor if the at least one FGFR2 genetic alteration or FGFR3 genetic alteration and the at least one FGFR1 genetic alteration are present in the sample, respectively.

The following methods for evaluating a biological sample for the presence of at least one FGFR2 or FGFR3 genetic alteration and at least one FGFR1, BRAF, EGFR, CCND1, ARID1A, ErbB2 or TERT genetic alterations, in particular for the presence of at least one FGFR2 or FGFR3 genetic alteration and at least one BRAF, EGFR, CCND1, ARID1A, ErbB2 or TERT genetic alterations, apply equally to any of the above disclosed methods of treatment and uses.

The disclosed methods are suitable for treating cancer generally, and mUC specifically, in a patient if at least one FGFR2 or FGFR3 genetic alterations and at least one FGFR1, BRAF, EGFR, CCND1, ARID1A, ErbB2 or TERT are present in a biological sample from the patient, in particular in a patient if at least one FGFR2 or FGFR3 genetic alterations and at least one BRAF, EGFR, CCND1, ARID1A, ErbB2 or TERT are present in a biological sample from the patient. In some embodiments, the genetic alteration can be one or more fusion genes. In some embodiments, the genetic alteration can be one or more mutations. In some embodiments, the genetic alteration can be one or more amplifications. In some embodiments, a combination of the one or more genetic alterations can be present in the biological sample from the patient.

For example, in some embodiments, the FGFR2 or FGFR3 genetic alterations can be one or more FGFR2 or FGFR3 fusion genes and one or more FGFR2 or FGFR3 mutations. Exemplary FGFR fusion genes are provided in Table 1 and include but are not limited to: FGFR2-BICC1; FGFR2-CASP7; FGFR3-BAIAP2L1; FGFR3-TACC3 V1; FGFR3-TACC3 V3; or a combination thereof.

For any of the genetic alterations identified herein, without intent to be limiting, evaluating a biological sample for the presence of at least one genetic alteration can comprise any combination of the following steps: isolating RNA from the biological sample; synthesizing cDNA from the RNA; and amplifying the cDNA (preamplified or non-preamplified). In some embodiments, evaluating a biological sample for the presence of at least one genetic alteration can comprise: amplifying cDNA from the patient with a pair of primers that bind to and amplify at least one genetic alteration; and determining whether the at least one genetic alteration is present in the sample. In some aspects, the cDNA can be pre-amplified. In some aspects, the evaluating step can comprise isolating RNA from the sample, synthesizing cDNA from the isolated RNA, and pre-amplifying the cDNA.

For FGFR specifically, suitable methods for evaluating a biological sample for the presence of one or more FGFR genetic alterations are described in the methods section herein and in WO 2016/048833 and U.S. patent application Ser. No. 16/723,975, which are incorporated herein in their entireties. Suitable primer pairs for performing an amplification step include, but are not limited to, those disclosed in WO 2016/048833, as exemplified below in Table 3:

TABLE 3

| Target | Forward Primer | Reverse Primer 5′-3′ |
|---|---|---|
| FGFR3-TACC3 V1 | GACCTGGACCGTGTCCTTACC | CTTCCCCAGTTCCAGGTTCTT |
| | (SEQ ID NO: 5) | (SEQ ID NO: 6) |
| FGFR3-TACC3 V3 | AGGACCTGGACCGTGTCCTT | TATAGGTCCGGTGGACAGGG |
| | (SEQ ID NO: 7) | (SEQ ID NO: 8) |
| FGFR3-BAIAP2L1 | CTGGACCGTGTCCTTACCGT | GCAGCCCAGGATTGAACTGT |
| | (SEQ ID NO: 9) | (SEQ ID NO: 10) |
| FGFR2-BICC1 | TGGATCGAATTCTCACTCTCACA | GCCAAGCAATCTGCGTATTTG |
| | (SEQ ID NO: 11) | (SEQ ID NO: 12) |
| FGFR2-CASP7 | GCTCTTCAATACAGCCCTGATCA | ACTTGGATCGAATTCTCACTCTCA |
| | (SEQ ID NO: 13) | (SEQ ID NO: 14) |
| FGFR2-CCDC6 | TGGATCGAATTCTCACTCTCACA | GCAAAGCCTGAATTTTCTTGAATAA |
| | (SEQ ID NO: 15) | (SEQ ID NO: 16) |
| FGFR3 R248C | GCATCCGGCAGACGTACA | CCCCGCCTGCAGGAT |
| | (SEQ ID NO: 17) | (SEQ ID NO: 18) |
| FGFR3 S249C | GCATCCGGCAGACGTACA | CCCCGCCTGCAGGAT |
| | (SEQ ID NO: 19) | (SEQ ID NO: 20) |
| FGFR3 G370C | AGGAGCTGGTGGAGGCTGA | CCGTAGCTGAGGATGCCTG |
| | (SEQ ID NO: 21) | (SEQ ID NO: 22) |
| FGFR3 Y373C | CTGGTGGAGGCTGACGAG | AGCCCACCCCGTAGCT |
| | (SEQ ID NO: 23) | (SEQ ID NO: 24) |
| FGFR3 R248C | GTCGTGGAGAACAAGTTTGGC | GTCTGGTTGGCCGGCAG |
| | (SEQ ID NO: 25) | (SEQ ID NO: 26) |
| FGFR3 S249C | GTCGTGGAGAACAAGTTTGGC | GTCTGGTTGGCCGGCAG |
| | (SEQ ID NO: 27) | (SEQ ID NO: 28) |
| FGFR3 G370C | AGGAGCTGGTGGAGGCTGA | CCGTAGCTGAGGATGCCTG |
| | (SEQ ID NO: 29) | (SEQ ID NO: 30) |
| FGFR3 Y373C | GACGAGGCGGGCAGTG | GAAGAAGCCCACCCCGTAG |
| | (SEQ ID NO: 31) | (SEQ ID NO: 32) |

The presence of any of the genetic alterations identified herein can be evaluated at any suitable time point including upon diagnosis, following tumor resection, following first-line therapy, during clinical treatment, or any combination thereof.

For example, a biological sample taken from a patient may be analyzed to determine whether a condition or disease, such as cancer, that the patient is or may be suffering from is one which is characterized by a genetic abnormality or abnormal protein expression which leads to upregulation of the levels or activity of FGFR2 or FGFR3 and BRAF, EGFR, CCND1, ARID1A, ErbB2 or TERT or to sensitization of a pathway to normal FGFR2 or FGFR3 and BRAF, EGFR, CCND1, ARID1A, ErbB2 or TERT activity. For example, a genetic abnormality or abnormal protein expression for FGFR2, FGFR3 or FGFR1 may lead to upregulation of these growth factor signaling pathways such as growth factor ligand levels or growth factor ligand activity or to upregulation of a biochemical pathway downstream of FGFR activation.

Examples of such abnormalities that result in activation or sensitization of the FGFR signal include loss of, or inhibition of apoptotic pathways, up-regulation of the receptors or ligands, or presence of genetic alterations of the receptors or ligands e.g. PTK variants. Tumors with genetic alterations of FGFR1, FGFR2 or FGFR3 or FGFR4 or up-regulation, in particular over-expression of FGFR1, or gain-of-function genetic alterations of FGFR2 or FGFR3 may be particularly sensitive to FGFR inhibitors.

The methods, drug products, and uses can further comprise evaluating the presence of at least one FGFR2 or FGFR3 genetic alteration and at least one FGFR1, BRAF, EGFR, CCND1, ARID1A, ErbB2 or TERT genetic alteration in the biological sample before the administering step.

In an embodiment, the presence of at least one FGFR2 genetic alteration or FGFR3 genetic alteration is evaluated in a sample from a cancer patient, in particular an urothelial carcinoma patient, before the cancer patient receives treatment with an FGFR inhibitor.

In an embodiment, the presence of at least one FGFR2 genetic alteration or FGFR3 genetic alteration and at least one EGFR, BRAF, CCND1, ARID1A, ErbB2, or TERT genetic alteration is evaluated in a sample from a cancer patient, in particular an urothelial carcinoma patient, before the cancer patient receives treatment with an FGFR inhibitor and a EGFR, BRAF, CCND1, ARID1A, ErbB2, or TERT inhibitor, respectively.

In an embodiment, the presence of at least one EGFR, BRAF, CCND1, ARID1A, ErbB2, or TERT genetic alteration is evaluated in a sample from a cancer patient, in particular an urothelial carcinoma patient, during treatment or after treatment of the cancer patient with an FGFR inhibitor. In an embodiment, the presence of at least one EGFR, BRAF, CCND1, ARID1A, ErbB2, or TERT genetic alteration is evaluated in a sample from a cancer patient, in particular an urothelial carcinoma patient, during treatment of the cancer patient with an FGFR inhibitor. In an embodiment, the presence of at least one EGFR, BRAF, CCND1, ARID1A, ErbB2, or TERT genetic alteration is evaluated in a sample from a cancer patient, in particular an urothelial carcinoma patient, after treatment of the cancer patient with an FGFR inhibitor.

In an embodiment, the presence of at least one FGFR2 genetic alteration or FGFR3 genetic alteration is evaluated in a sample from a cancer patient, in particular an urothelial carcinoma patient, before the cancer patient receives treatment with an FGFR inhibitor, and the presence of at least one EGFR, BRAF, CCND1, ARID1A, ErbB2, or TERT genetic alteration is evaluated in a sample from the cancer patient, in particular the urothelial carcinoma patient, during treatment or after treatment of the cancer patient with an FGFR inhibitor. In an embodiment, the presence of at least one FGFR2 genetic alteration or FGFR3 genetic alteration is evaluated in a sample from a cancer patient, in particular an urothelial carcinoma patient, before the cancer patient receives treatment with an FGFR inhibitor, and the presence of at least one EGFR, BRAF, CCND1, ARID1A, ErbB2, or TERT genetic alteration is evaluated in a sample from the cancer patient, in particular the urothelial carcinoma patient, during treatment of the cancer patient with an FGFR inhibitor. In an embodiment, the presence of at least one FGFR2 genetic alteration or FGFR3 genetic alteration is evaluated in a sample from a cancer patient, in particular an urothelial carcinoma patient, before the cancer patient receives treatment with an FGFR inhibitor, and the presence of at least one EGFR, BRAF, CCND1, ARID1A, ErbB2, or TERT genetic alteration is evaluated in a sample from the cancer patient, in particular the urothelial carcinoma patient, after treatment of the cancer patient with an FGFR inhibitor.

In an embodiment, the presence of at least one FGFR2 genetic alteration or FGFR3 genetic alteration is evaluated in a sample from a cancer patient, in particular an urothelial carcinoma patient, before the cancer patient receives treatment with an FGFR inhibitor and when at least one FGFR2 genetic alteration or FGFR3 genetic alteration is present in the sample from the cancer patient, the cancer patient receives treatment with an FGFR inhibitor, and the presence of at least one EGFR, BRAF, CCND1, ARID1A, ErbB2, or TERT genetic alteration is evaluated in a sample from the cancer patient, in particular the urothelial carcinoma patient, during treatment or after treatment of the cancer patient with the FGFR inhibitor. In an embodiment, the presence of at least one FGFR2 genetic alteration or FGFR3 genetic alteration is evaluated in a sample from a cancer patient, in particular an urothelial carcinoma patient, before the cancer patient receives treatment with an FGFR inhibitor and when at least one FGFR2 genetic alteration or FGFR3 genetic alteration is present in the sample from the cancer patient, the cancer patient receives treatment with an FGFR inhibitor, and the presence of at least one EGFR, BRAF, CCND1, ARID1A, ErbB2, or TERT genetic alteration is evaluated in a sample from the cancer patient, in particular the urothelial carcinoma patient, during treatment the cancer patient with the FGFR inhibitor. In an embodiment, the presence of at least one FGFR2 genetic alteration or FGFR3 genetic alteration is evaluated in a sample from a cancer patient, in particular an urothelial carcinoma patient, before the cancer patient receives treatment with an FGFR inhibitor and when at least one FGFR2 genetic alteration or FGFR3 genetic alteration is present in the sample from the cancer patient, the cancer patient receives treatment with an FGFR inhibitor, and the presence of at least one EGFR, BRAF, CCND1, ARID1A, ErbB2, or TERT genetic alteration is evaluated in a sample from the cancer patient, in particular the urothelial carcinoma patient, after treatment of the cancer patient with the FGFR inhibitor.

In an embodiment, the presence of at least one FGFR2 genetic alteration or FGFR3 genetic alteration is evaluated in a sample from a cancer patient, in particular an urothelial carcinoma patient, before the cancer patient receives treatment with an FGFR inhibitor and when at least one FGFR2 genetic alteration or FGFR3 genetic alteration is present in the sample from the cancer patient, the cancer patient receives treatment with an FGFR inhibitor, and the presence of at least one EGFR, BRAF, CCND1, ARID1A, ErbB2, or TERT genetic alteration is evaluated in a sample from the cancer patient, in particular the urothelial carcinoma patient, during treatment or after treatment of the cancer patient with the FGFR inhibitor, and when at least one EGFR, BRAF, CCND1, ARID1A, ErbB2, or TERT genetic alteration is present in the sample, the patient receives treatment with an EGFR, BRAF, CCND1, ARID1A, ErbB2, or TERT inhibitor, respectively. In an embodiment, the presence of at least one FGFR2 genetic alteration or FGFR3 genetic alteration is evaluated in a sample from a cancer patient, in particular an urothelial carcinoma patient, before the cancer patient receives treatment with an FGFR inhibitor and when at least one FGFR2 genetic alteration or FGFR3 genetic alteration is present in the sample from the cancer patient, the cancer patient receives treatment with an FGFR inhibitor, and the presence of at least one EGFR, BRAF, CCND1, ARID1A, ErbB2, or TERT genetic alteration is evaluated in a sample from the cancer patient, in particular the urothelial carcinoma patient, during treatment of the cancer patient with the FGFR inhibitor, and when at least one EGFR, BRAF, CCND1, ARID1A, ErbB2, or TERT genetic alteration is present in the sample, the patient receives treatment with an EGFR, BRAF, CCND1, ARID1A, ErbB2, or TERT inhibitor, respectively. In an embodiment, the presence of at least one FGFR2 genetic alteration or FGFR3 genetic alteration is evaluated in a sample from a cancer patient, in particular an urothelial carcinoma patient, before the cancer patient receives treatment with an FGFR inhibitor and when at least one FGFR2 genetic alteration or FGFR3 genetic alteration is present in the sample from the cancer patient, the cancer patient receives treatment with an FGFR inhibitor, and the presence of at least one EGFR, BRAF, CCND1, ARID1A, ErbB2, or TERT genetic alteration is evaluated in a sample from the cancer patient, in particular the urothelial carcinoma patient, after treatment of the cancer patient with the FGFR inhibitor, and when at least one EGFR, BRAF, CCND1, ARID1A, ErbB2, or TERT genetic alteration is present in the sample, the patient receives treatment with an EGFR, BRAF, CCND1, ARID1A, ErbB2, or TERT inhibitor, respectively.

The diagnostic tests and screens are typically conducted on a biological sample selected from tumor biopsy samples, blood samples (isolation and enrichment of shed tumor cells), stool biopsies, sputum, chromosome analysis, pleural fluid, peritoneal fluid, buccal spears, biopsy, circulating DNA, or urine. In certain embodiments, the biological sample is blood, lymph fluid, bone marrow, a solid tumor sample, or any combination thereof. In certain embodiments, the biological sample is a solid tumor sample. In certain embodiments, the biological sample is a blood sample. In certain embodiments, the biological sample is a urine sample.

Methods of identification and analysis of genetic alterations and up-regulation of proteins are known to a person skilled in the art. Screening methods could include, but are not limited to, standard methods such as reverse-transcriptase polymerase chain reaction (RT PCR) or in-situ hybridization such as fluorescence in situ hybridization (FISH).

Identification of an individual carrying at least one FGFR2 or FGFR3 genetic alteration and at least one BRAF, EGFR, CCND1, ARID1A, ErbB2 or TERT genetic alteration as described herein, may mean that the patient would be particularly suitable for treatment with erdafitinib in combination with a BRAF, EGFR, CCND1, ARID1A, ErbB2 or TERT inhibitor, respectively. Tumors may preferentially be screened for presence of a genetic variant prior to treatment. The screening process will typically involve direct sequencing, oligonucleotide microarray analysis, or a mutant specific antibody. In addition, diagnosis of tumor with such genetic alteration could be performed using techniques known to a person skilled in the art and as described herein such as RT-PCR and FISH.

In addition, genetic alterations of, for example FGFR, can be identified by direct sequencing of, for example, tumor biopsies using PCR and methods to sequence PCR products directly as hereinbefore described. The skilled artisan will recognize that all such well-known techniques for detection of the over expression, activation or mutations of the aforementioned proteins could be applicable in the present case.

In screening by RT-PCR, the level of mRNA in the tumor is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. (2004) Current Protocols in Molecular Biology, John Wiley & Sons Inc., or Innis, M. A. et al., eds. (1990) PCR Protocols: a guide to methods and applications, Academic Press, San Diego. Reactions and manipulations involving nucleic acid techniques are also described in Sambrook et al., (2001), 3rd Ed, Molecular Cloning: A Laboratory Manual, Cold Spring Harbor Laboratory Press. Alternatively, a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference. An example of an in-situ hybridization technique for assessing mRNA expression would be fluorescence in-situ hybridization (FISH) (see Angerer (1987) Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labelled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. (2004) Current Protocols in Molecular Biology, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Methods for gene expression profiling are described by (DePrimo et al. (2003), BMC Cancer, 3:3). Briefly, the protocol is as follows: double-stranded cDNA is synthesized from total RNA Using a (dT)24 oligomer for priming first-strand cDNA synthesis, followed by second strand cDNA synthesis with random hexamer primers. The double-stranded cDNA is used as a template for in vitro transcription of cRNA using biotinylated ribonucleotides. cRNA is chemically fragmented according to protocols described by Affymetrix (Santa Clara, CA, USA), and then hybridized overnight on Human Genome Arrays.

Alternatively, the protein products expressed from the mRNAs may be assayed by immunohistochemistry of tumor samples, solid phase immunoassay with microtitre plates, Western blotting, 2-dimensional SDS-polyacrylamide gel electrophoresis, ELISA, flow cytometry and other methods known in the art for detection of specific proteins. Detection methods would include the use of site-specific antibodies. The skilled person will recognize that all such well-known techniques for detection of upregulation of protein levels or detection of genetic variants or mutants could be applicable in the present case.

Abnormal levels of proteins such as FGFR can be measured using standard enzyme assays, for example, those assays described herein. Activation or overexpression could also be detected in a tissue sample, for example, a tumor tissue by measuring the tyrosine kinase activity with an assay such as that from Chemicon International. The tyrosine kinase of interest would be immunoprecipitated from the sample lysate and its activity measured.

For FGF2, FGFR3 or FGFR1 genetic alterations specifically, alternative methods for the measurement of the over expression or activation of FGFR including the isoforms thereof, include the measurement of microvessel density. This can for example be measured using methods described by Orre and Rogers (Int J Cancer (1999), 84(2) 101-8). Assay methods also include the use of markers.

Therefore, all of these techniques could also be used to identify tumors particularly suitable for treatment with the compounds of the invention.

Erdafitinib is in particular useful in treatment of a patient having a genetic altered FGFR, in particular a mutated FGFR or a FGFR fusion. In certain embodiments, the urothelial carcinoma is susceptible to an FGFR2 genetic alteration and/or an FGFR3 genetic alteration. In certain embodiments, the FGFR2 or FGFR3 genetic alteration is an FGFR3 gene mutation or an FGFR2 or FGFR3 gene fusion. In some embodiments, the FGFR3 gene mutation is R248C, S249C, G370C, Y373C, or any combination thereof. In further embodiments, the FGFR2 or FGFR3 gene fusion is FGFR3-TACC3, in particular FGFR3-TACC3 V1 or V3, FGFR3-BAIAP2L1, FGFR2-BICC1, FGFR2-CASP7, or any combination thereof.

According to certain embodiments, FGFR2 and/or FGFR3 genetic alterations can be identified using commercially available kits including, but not limited to, the QIAGEN Therascreen® FGFR RGQ RT-PCR kit. According to certain embodiments, the BRAF, EGFR, ARID1A, ERBB2 and TERT genetic alterations can be identified using commercially available kits including, but not limited to, the GUARDANT360® assay.

### FGFR Inhibitor Pharmaceutical Compositions and Routes of Administration

In view of its useful pharmacological properties, the FGFR inhibitor generally, and erdafitinib more specifically, may be formulated into various pharmaceutical forms for administration purposes.

In one embodiment the pharmaceutical composition (e.g. formulation) comprises at least one active compound according to the invention, in particular an FGFR inhibitor, together with one or more pharmaceutically acceptable carriers, adjuvants, excipients, diluents, fillers, buffers, stabilizers, preservatives, lubricants, or other materials well known to those skilled in the art and optionally other therapeutic or prophylactic agents.

To prepare the pharmaceutical compositions, an effective amount of the FGFR inhibitor generally, and erdafitinib more specifically, as the active ingredient is combined in intimate admixture with a pharmaceutically acceptable carrier, which carrier may take a wide variety of forms depending on the form of preparation desired for administration. The pharmaceutical compositions can be in any form suitable for oral, parenteral, topical, intranasal, ophthalmic, otic, rectal, intra-vaginal, or transdermal administration. These pharmaceutical compositions are desirably in unitary dosage form suitable, preferably, for administration orally, rectally, percutaneously, or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets.

The pharmaceutical compositions of the invention, in particular capsules and/or tablets, may include one or more pharmaceutically acceptable excipients (pharmaceutically acceptable carrier) such as disintegrants, diluents, fillers, binders, buffering agents, lubricants, glidants, thickening agents, sweetening agents, flavors, colorants, preservatives and the like. Some excipients can serve multiple purposes.

Suitable disintegrants are those that have a large coefficient of expansion. Examples thereof are hydrophilic, insoluble or poorly water-soluble crosslinked polymers such as crospovidone (crosslinked polyvinylpyrrolidone) and croscarmellose sodium (crosslinked sodium carboxymethylcellulose). The amount of disintegrant in the tablets according to the present invention may conveniently range from about 2.5 to about 15% w/w and preferably range from about 2.5 to 7% w/w, in particular range from about 2.5 to 5% w/w. Because disintegrants by their nature yield sustained release formulations when employed in bulk, it is advantageous to dilute them with an inert substance called a diluent or filler.

A variety of materials may be used as diluents or fillers. Examples are lactose monohydrate, anhydrous lactose, sucrose, dextrose, mannitol, sorbitol, starch, cellulose (e.g. micro-crystalline cellulose (Avicel™), silicified microcrystalline cellulose), dihydrated or anhydrous dibasic calcium phosphate, and others known in the art, and mixtures thereof (e.g. spray-dried mixture of lactose monohydrate (75%) with microcrystalline cellulose (25%) which is commercially available as Microcelac™). Preferred are microcrystalline cellulose and mannitol. The total amount of diluent or filler in the pharmaceutical compositions of the present invention may conveniently range from about 20% to about 95% w/w and preferably ranges from about 55% to about 95% w/w, or from about 70% to about 95% w/w, or from about 80% to about 95% w/w, or from about 85% to about 95% w/w.

Lubricants and glidants can be employed in the manufacture of certain dosage forms and will usually be employed when producing tablets. Examples of lubricants and glidants are hydrogenated vegetable oils, e.g hydrogenated Cottonseed oil, magnesium stearate, stearic acid, sodium lauryl sulfate, magnesium lauryl sulfate, colloidal silica, colloidal anhydrous silica, talc, mixtures thereof, and others known in the art. Interesting lubricants are magnesium stearate, and mixtures of magnesium stearate with colloidal silica, magnesium stearate being preferred. A preferred glidant is colloidal anhydrous silica.

If present, glidants generally comprise 0.2 to 7.0% w/w of the total composition weight, in particular 0.5 to 1.5% w/w, more in particular 1 to 1.5% w/w.

If present, lubricants generally comprise 0.2 to 7.0% w/w of the total composition weight, in particular 0.2 to 2% w/w, or 0.5 to 2% w/w, or 0.5 to 1.75% w/w, or 0.5 to 1.5% w/w.

Binders can optionally be employed in the pharmaceutical compositions of the present invention. Suitable binders are water-soluble polymers, such as alkylcelluloses such as methylcellulose; hydroxyalkylcelluloses such as hydroxymethylcellulose, hydroxyethylcellulose, hydroxypropylcellulose and hydroxybutylcellulose; hydroxyalkyl alkylcelluloses such as hydroxyethyl methylcellulose and hydroxypropyl methylcellulose; carboxyalkylcelluloses such as carboxymethylcellulose; alkali metal salts of carboxyalkylcelluloses such as sodium carboxymethylcellulose; carboxyalkylalkylcelluloses such as carboxymethylethylcellulose; carboxyalkylcellulose esters; starches; pectines such as sodium carboxymethylamylopectine; chitin derivates such as chitosan; di-, oligo- and polysaccharides such as trehalose, cyclodextrins and derivatives thereof, alginic acid, alkali metal and ammonium salts thereof, carrageenans, galactomannans, tragacanth, agar agar, gummi arabicum, guar gummi and xanthan gummi; polyacrylic acids and the salts thereof; polymethacrylic acids, the salts and esters thereof, methacrylate copolymers; polyvinylpyrrolidone (PVP), polyvinylalcohol (PVA) and copolymers thereof, e.g. PVP-VA. Preferably, the water-soluble polymer is a hydroxyalkyl alkylcelluloses, such as for example hydroxypropylmethyl cellulose, e.g. hydroxypropylmethyl cellulose 15 cps.

Other excipients such as coloring agents and pigments may also be added to the compositions of the invention. Coloring agents and pigments include titanium dioxide and dyes suitable for food. A coloring agent or a pigment is an optional ingredient in the formulation of the invention, but when used the coloring agent can be present in an amount up to 3.5% w/w based on the total composition weight.

Flavors are optional in the composition and may be chosen from synthetic flavor oils and flavoring aromatics or natural oils, extracts from plants leaves, flowers, fruits and so forth and combinations thereof. These may include cinnamon oil, oil of wintergreen, peppermint oils, bay oil, anise oil, eucalyptus, thyme oil. Also useful as flavors are vanilla, citrus oil, including lemon, orange, grape, lime and grapefruit, and fruit essences, including apple, banana, pear, peach, strawberry, raspberry, cherry, plum, pineapple, apricot and so forth, the amount of flavor may depend on a number of factors including the organoleptic effect desired. Generally, the flavor will be present in an amount from about 0% to about 3% (w/w).

Formaldehyde scavengers are compounds that are capable of absorbing formaldehyde. They include compounds comprising a nitrogen center that is reactive with formaldehyde, such as to form one or more reversible or irreversible bonds between the formaldehyde scavenger and formaldehyde. For example, the formaldehyde scavenger comprises one or more nitrogen atoms/centers that are reactive with formaldehyde to form a schiff base imine that is capable of subsequently binding with formaldehyde. For example, the formaldehyde scavenger comprises one or more nitrogen centers that are reactive with formaldehyde to form one or more 5-8 membered cyclic rings. The formaldehyde scavenger preferably comprises one or more amine or amide groups. For example, the formaldehyde scavenger can be an amino acid, an amino sugar, an alpha amine compound, or a conjugate or derivative thereof, or a mixture thereof. The formaldehyde scavenger may comprise two or more amines and/or amides.

Formaldehyde scavengers include, for example, glycine, alanine, serine, threonine, cysteine, valine, lecuine, isoleucine, methionine, phenylalanine, tyrosine, aspartic acid, glutamic acid, arginine, lysine, ornithine, citrulline, taurine pyrrolysine, meglumine, histidine, aspartame, proline, tryptophan, citrulline, pyrrolysine, asparagine, glutamine, or a conjugate or mixture thereof, or, whenever possible, pharmaceutically acceptable salts thereof.

In an aspect of the invention, the formaldehyde scavenger is meglumine or a pharmaceutically acceptable salt thereof, in particular meglumine base.

In an embodiment, in the methods and uses as described herein, erdafitinib is administered or is to be administered as a pharmaceutical composition, in particular a tablet or capsule, comprising erdafitinib or a pharmaceutically acceptable salt thereof, in particular erdafitinib base; a formaldehyde scavenger, in particular meglumine or a pharmaceutically acceptable salt thereof, in particular meglumine base; and a pharmaceutically acceptable carrier.

It is another object of the invention to provide a process of preparing a pharmaceutical composition as described herein, in particular in the form of a tablet or a capsule, characterized by blending a formaldehyde scavenger, in particular meglumine, and erdafitinib, a pharmaceutically acceptable salt thereof or a solvate thereof, in particular erdafitinib base, with a pharmaceutically acceptable carrier and compressing said blend into tablets or filling said blend in capsules.

Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, to aid solubility for example, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wetting agent, optionally combined with suitable additives of any nature in minor proportions, which additives do not cause a significant deleterious effect to the skin. Said additives may facilitate the administration to the skin and/or may be helpful for preparing the desired compositions. These compositions may be administered in various ways, e.g., as a transdermal patch, as a spot-on, as an ointment. It is especially advantageous to formulate the aforementioned pharmaceutical compositions in dosage unit form for ease of administration and uniformity of dosage. Dosage unit form as used in the specification refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof. Preferred forms are tablets and capsules.

In certain embodiments, the FGFR inhibitor is present in a solid unit dosage form, and a solid unit dosage form suitable for oral administration. The unit dosage form may contain about 1, 2, 3, 4, 5, 6, 7, 8, 9, or 10 mg of the FGFR inhibitor per unit dose form or an amount in a range bounded by two of these values, in particular 3, 4 or 5 mg per unit dose.

Depending on the mode of administration, the pharmaceutical composition will preferably comprise from 0.05 to 99% by weight, more preferably from 0.1 to 70% by weight, even more preferably from 0.1 to 50% by weight of the compound according to the present invention, in particular an FGFR inhibitor, and, from 1 to 99.95% by weight, more preferably from 30 to 99.9% by weight, even more preferably from 50 to 99.9% by weight of a pharmaceutically acceptable carrier, all percentages being based on the total weight of the composition.

Tablets or capsules of the present invention may further be film-coated e.g. to improve taste, to provide ease of swallowing and an elegant appearance. Polymeric film-coating materials are known in the art. Preferred film coatings are water-based film coatings opposed to solvent based film coatings because the latter may contain more traces of aldehydes. A preferred film-coating material is Opadry® II aqueous film coating system, e.g. Opadry® II 85F, such as Opadry® II 85F92209. Further preferred film coatings are water-based film coatings that protects from environmental moisture, such as Readilycoat® (e.g. Readilycoat® D), AquaPolish® MS, Opadry® amb, Opadry® amb II, which are aqueous moisture barrier film coating systems. A preferred film-coating is Opadry® amb II, a high-performance moisture barrier film coating which is a PVA-based immediate release system, without polyethylene glycol.

In tablets according to the invention, the film coat in terms of weight preferably accounts for about 4% (w/w) or less of the total tablet weight.

For capsules according to the present invention, hypromellose (HPMC) capsules are preferred over gelatin capsules.

In an aspect of the invention, the pharmaceutical compositions as described herein, in particular in the form of a capsule or a tablet, comprise from 0.5 mg to 20 mg base equivalent, or from 2 mg to 20 mg base equivalent, or from 0.5 mg to 12 mg base equivalent, or from 2 mg to 12 mg base equivalent, or from 2 mg to 10 mg base equivalent, or from 2 mg to 6 mg base equivalent, or 2 mg base equivalent, 3 mg base equivalent, 4 mg base equivalent, 5 mg base equivalent, 6 mg base equivalent, 7 mg base equivalent, 8 mg base equivalent, 9 mg base equivalent, 10 mg base equivalent, 11 mg base equivalent or 12 mg base equivalent of erdafitinib, a pharmaceutically acceptable salt thereof or a solvate thereof. In particular, the pharmaceutical compositions as described herein comprise 3 mg base equivalent, 4 mg base equivalent or 5 mg base equivalent of erdafitinib, a pharmaceutically acceptable salt thereof or a solvate thereof, in particular 3 mg or 4 mg or 5 mg of erdafitinib base.

In an aspect of the invention, the pharmaceutical compositions as described herein, in particular in the form of a capsule or a tablet, comprise from 0.5 mg to 20 mg, or from 2 mg to 20 mg, or from 0.5 mg to 12 mg, or from 2 mg to 12 mg, or from 2 mg to 10 mg, or from 2 mg to 6 mg, or 2 mg, 3 mg, 4 mg, 5 mg, 6 mg, 7 mg, 8 mg, 9 mg, 10 mg, 11 mg or 12 mg of erdafitinib base. In particular, the pharmaceutical compositions as described herein comprise 3 mg, 4 mg or 5 mg of erdafitinib base. In particular, the pharmaceutical compositions as described herein comprise 3 mg, 4 mg or 5 mg of erdafitinib base and from about 0.5 to about 5% w/w, from about 0.5 to about 3% w/w, from about 0.5 to about 2% w/w, from about 0.5 to about 1.5% w/w, or from about 0.5 to about 1% w/w of a formaldehyde scavenger, in particular meglumine. In particular, the pharmaceutical compositions as described herein comprise 3 mg, 4 mg or 5 mg of erdafitinib base and from about 0.5 to about 1.5% w/w or from about 0.5 to about 1% w/w of a formaldehyde scavenger, in particular meglumine, more in particular meglumine base.

In an aspect of the invention, more than one, e.g. two, pharmaceutical compositions as described herein can be administered in order to obtain a desired dose, e.g. a daily dose. For example, for a daily dose of 8 mg base equivalent of erdafitinib, 2 tablets or capsules of 4 mg erdafitinib base equivalent each may be administered; or a tablet or a capsule of 3 mg erdafitinib base equivalent and a tablet or capsule of 5 mg base equivalent may be administered. For example, for a daily dose of 9 mg base equivalent of erdafitinib, 3 tablets or capsules of 3 mg erdafitinib base equivalent each may be administered; or a tablet or a capsule of 4 mg erdafitinib base equivalent and a tablet or capsule of 5 mg base equivalent may be administered.

The amount of formaldehyde scavenger, in particular meglumine, in the pharmaceutical compositions according to the present invention may range from about 0.1 to about 10% w/w, about 0.1 to about 5% w/w, from about 0.1 to about 3% w/w, from about 0.1 to about 2% w/w, from about 0.1 to about 1.5% w/w, from about 0.1 to about 1% w/w, from about 0.5 to about 5% w/w, from about 0.5 to about 3% w/w, from about 0.5 to about 2% w/w, from about 0.5 to about 1.5% w/w, from about 0.5 to about 1% w/w.

According to particular embodiments, erdafitinib is supplied as 3 mg, 4 mg or 5 mg film-coated tablets for oral administration and contains the following inactive ingredients or equivalents thereof: Tablet Core: croscarmellose sodium, magnesium stearate, mannitol, meglumine, and microcrystalline cellulose; and Film Coating: Opadry amb II: Glycerol monocaprylocaprate Type I, polyvinyl alcohol-partially hydrolyzed, sodium lauryl sulfate, talc, titanium dioxide, iron oxide yellow, iron oxide red (for orange and brown tablets), ferrosoferric oxide/iron oxide black (for brown tablets).

Studies that look at safety seek to identify any potential adverse effects that may result from exposure to the drug. Efficacy is often measured by determining whether an active pharmaceutical ingredient demonstrates a health benefit over a placebo or other intervention when tested in an appropriate situation, such as a tightly controlled clinical trial.

The term “acceptable” with respect to a formulation, composition or ingredient, as used herein, means that the beneficial effects of that formulation, composition or ingredient on the general health of the human being treated substantially outweigh its detrimental effects, to the extent any exist.

All formulations for oral administration are in dosage form suitable for such administration.

Bispecific EGFR c-Met Antibody Pharmaceutical Compositions and Routes of Administration

The invention provides for pharmaceutical compositions comprising the bispecific EGFR/c-Met antibody (e.g., amivantamab) disclosed herein and a pharmaceutically acceptable carrier. The bispecific anti-EGFR/c-Met antibody, in particular amivantamab, may be formulated at 50 mg/mL to up to 450 mg/mL into a pharmaceutical composition comprising the bispecific anti-EGFR/c-Met antibody and a pharmaceutically acceptable carrier. The pharmaceutically acceptable carrier may be one or more diluents, adjuvants, excipients, vehicles and the like. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used to formulate the bispecific anti-EGFR/c-Met antibody. The formulation may also contain an agent to facilitate subcutaneous injection such as a recombinant human hyaluronidase. These solutions are sterile and generally free of particulate matter. They may be sterilized by conventional, well-known sterilization techniques (e.g., filtration). In some embodiments, the bispecific anti-EGFR/c-Met antibody is administered by an intravenous injection. In some embodiments, the bispecific anti-EGFR/c-Met antibody is administered by a subcutaneous injection.

### Methods of Dosing and Treatment Regimens

Disclosed herein are methods of treating cancer generally, and mUC specifically, comprising, consisting of, or consisting essentially of, administering an FGFR inhibitor in combination with another FGFR inhibitor or a BRAF, EGFR, CCND1, ARID1A, ErbB2 or TERT inhibitor to a patient in need of cancer treatment generally, and mUC treatment more specifically, wherein the patient harbors at least one FGFR2 genetic alteration or FGFR3 genetic alteration and at least one FGFR1, BRAF, EGFR, CCND1, ARID1A, ErbB2 or TERT genetic alteration, respectively, wherein the FGFR inhibitor is preferably administered orally. In some embodiments, the FGFR inhibitor generally, and erdafitinib specifically, is administered daily, in particular once daily. In some embodiments, the FGFR inhibitor generally, and erdafitinib specifically, is administered twice-a-day. In some embodiments, the FGFR inhibitor generally, and erdafitinib specifically, is administered three times a day. In some embodiments, the FGFR inhibitor generally, and erdafitinib specifically, is administered four times a day. In some embodiments, the FGFR inhibitor generally, and erdafitinib specifically, is administered every other day. In some embodiments, the FGFR inhibitor generally, and erdafitinib specifically, is administered weekly. In some embodiments, the FGFR inhibitor generally, and erdafitinib specifically is administered twice a week. In some embodiments, the FGFR inhibitor generally, and erdafitinib specifically, is administered every other week. In some embodiments, the FGFR inhibitor generally, and erdafitinib specifically, is administered orally on a continuous daily dosage schedule.

In general, doses of the FGFR inhibitor, and erdafitinib specifically, employed for treatment of the diseases or conditions described herein in humans are typically in the range of about 1 to 20 mg per day. In some embodiments, the FGFR inhibitor, and erdafitinib specifically, is administered orally to the human at a dose of about 1 mg per day, about 2 mg per day, about 3 mg per day, about 4 mg per day, about 5 mg per day, about 6 mg per day, about 7 mg per day, about 8 mg per day, about 9 mg per day, about 10 mg per day, about 11 mg per day, about 12 mg per day, about 13 mg per day, about 14 mg per day, about 15 mg per day, about 16 mg per day, about 17 mg per day, about 18 mg per day, about 19 mg per day or about 20 mg per day.

In some embodiments, erdafitinib is administered orally. In certain embodiments, erdafitinib is administered orally at a dose of about 8 mg once daily. In further embodiments, the dose of erdafitinib is increased from 8 mg once daily to 9 mg once daily. In still further embodiments, the dose of erdafitinib is increased from 8 mg once daily to 9 mg once daily if: (a) the patient exhibits a serum phosphate (P04) level that is less than about 5.5 mg/dL at 14-21 days after initiating treatment; and (b) administration of erdafitinib at 8 mg once daily resulted in no ocular disorder; or (c) administration of erdafitinib at 8 mg once daily resulted in no Grade 2 or greater adverse reaction, wherein the increase from 8 mg once daily to 9 mg once daily begins at 14 to 21 days after initiating treatment.

In certain embodiments, the dose of erdafitinib is increased from 8 mg once daily to 9 mg once daily at 14 days after initiating treatment. In certain embodiments, the dose of erdafitinib is increased from 8 mg once daily to 9 mg once daily at 15 days after initiating treatment. In certain embodiments, the dose of erdafitinib is increased from 8 mg once daily to 9 mg once daily at 16 days after initiating treatment. In certain embodiments, the dose of erdafitinib is increased from 8 mg once daily to 9 mg once daily at 17 days after initiating treatment. In certain embodiments, the dose of erdafitinib is increased from 8 mg once daily to 9 mg once daily at 18 days after initiating treatment. In certain embodiments, the dose of erdafitinib is increased from 8 mg once daily to 9 mg once daily at 19 days after initiating treatment. In certain embodiments, the dose of erdafitinib is increased from 8 mg once daily to 9 mg once daily at 20 days after initiating treatment. In certain embodiments, the dose of erdafitinib is increased from 8 mg once daily to 9 mg once daily at 21 days after initiating treatment

In an embodiment, erdafitinib is administered at a dose of 8 mg, in particular 8 mg once daily. In an embodiment, erdafitinib is administered at a dose of 8 mg, in particular 8 mg once daily, with an option to uptitrate to 9 mg depending on serum phosphate levels (e.g. serum phosphate levels are <5.5 mg/dL, or are <7 mg/dL or range from and include 7 mg/dL to ≤9 mg/dL or are ≤9 mg/dL), and depending on treatment-related adverse events observed. In an embodiment, the levels of serum phosphate for determining whether or not to up-titrate are measured on a treatment day during the first cycle of erdafitinib treatment, in particular on day 14±2 days, more in particular on day 14, of erdafitinib administration.

In an embodiment, the treatment cycle as used herein is a 28-day cycle. In certain embodiments, the treatment cycle is a 28-day cycle for up to two years. In certain embodiments, the treatment cycle is four weeks.

In one embodiment, the desired dose is conveniently presented in a single dose or in divided doses administered simultaneously (or over a short period of time) or at appropriate intervals, for example as two, three, four or more sub-doses per day. In some embodiments, the FGFR inhibitor is conveniently presented in divided doses that are administered simultaneously (or over a short period of time) once a day. In some embodiments, the FGFR inhibitor generally, and erdafitinib specifically, is conveniently presented in divided doses that are administered in equal portions twice-a-day. In some embodiments, the FGFR inhibitor generally, and erdafitinib specifically, is conveniently presented in divided doses that are administered in equal portions three times a day. In some embodiments, the FGFR inhibitor, and erdafitinib specifically, is conveniently presented in divided doses that are administered in equal portions four times a day.

In certain embodiments, the desired dose may be delivered in 1, 2, 3, 4, 5, 6, 7, 8, 9, or 10 fractional unit dosages throughout the course of the day, such that the total amount of FGFR inhibitor generally, and erdafitinib specifically, delivered by the fractional unit dosages over the course of the day provides the total daily dosages.

In some embodiments, the amount of the FGFR inhibitor generally, and erdafitinib specifically, that is given to the human varies depending upon factors such as, but not limited to, condition and severity of the disease or condition, and the identity (e.g., weight) of the human, and the particular additional therapeutic agents that are administered (if applicable).

The bispecific EGFR/c-Met antibodies described herein, in particular amivantamab, may be administered to a patient by any suitable route, for example parentally by intravenous (IV) infusion or bolus injection, intramuscularly or subcutaneously or intraperitoneally. IV infusion can be given over as little as 15 minutes, but more often for 30 minutes, 60 minutes, 90 minutes, 2 hours, 3 hours or even 7 to 8 hours. The initial administration may also be a split infusion over 2 days. The bispecific EGFR/c-Met antibodies may also be injected directly into the site of disease (e.g., the tumor itself). The dose given to a patient having a cancer is sufficient to alleviate or at least partially arrest the disease being treated (“therapeutically effective amount”) and may be sometimes 0.1 to 10 mg/kg body weight, for example 1, 2, 3, 4, 5, 6, 7, 8, 9 or 10 mg/kg, but may be even higher, for example 15, 17.5, 20, 30, 40, 50, 60, 70, 80, 90 or 100 mg/kg. A fixed unit dose may also be given, for example, 50, 100, 200, 500, 1000, 1050, 1400 mg, or 1700 to 1800 mg or the dose may be based on the patient's surface area, e.g., 400, 300, 250, 200, or 100 mg/m². Usually between 1 and 8 doses, (e.g., 1, 2, 3, 4, 5, 6, 7 or 8) may be administered to treat cancer, but 10, 12, 20 or more doses may be given. Administration of the bispecific EGFR/c-Met antibody of the invention, in particular amivantamab, may be repeated after one day, two days, three days, four days, five days, six days, one week, two weeks, three weeks, one month, five weeks, six weeks, seven weeks, two months, three months, four months, five months, six months or longer, including weekly for four weeks, then every week thereafter. Repeated courses of treatment are also possible, as is chronic administration. The repeated administration may be at the same dose or at a different dose.

In one specific embodiment, the FGFR inhibitor is administered in combination with a BRAF, EGFR, CCND1, ARID1A, ERBB2 or TERT inhibitor, wherein the FGFR inhibitor and the BRAF, EGFR, CCND1, ARID1A, ErbB2 or TERT inhibitor modulate different aspects of the cancer generally, and mUC specifically, thereby providing a greater overall benefit than administration of either therapeutic agent alone.

The overall benefit experienced by the patient may simply be additive of the two therapeutic agents or the patient may experience a synergistic benefit.

In certain embodiments, the patient received treatment with an FGFR inhibitor generally, and erdafitinib more specifically, prior to co-administering the FGFR inhibitor generally, and erdafitinib more specifically, with the at least one BRAF, EGFR, CCND1, ARID1A, ErbB2 or TERT inhibitor, or with another FGFR inhibitor.

Described herein method of treating cancer in a patient comprising: (a) evaluating a biological sample from the patient for the presence of at least one FGFR2 genetic alteration or FGFR3 genetic alteration and at least one BRAF, EGFR, CCND1, ARID1A, ErbB2 or TERT genetic alteration; and (b) treating the patient with an FGFR inhibitor in combination with a BRAF, EGFR, CCND1, ARID1A, ErbB2 or TERT inhibitor if the at least one FGFR2 genetic alteration or FGFR3 genetic alteration and the at least one BRAF, EGFR, CCND1, ARID1A, ErbB2 or TERT genetic alteration, respectively, are present in the sample. In certain embodiments, the patient is treated with an FGFR inhibitor prior to step (a) of evaluating a biological sample from the patient for the presence of at least one BRAF, EGFR, CCND1, ARID1A, ErbB2 or TERT genetic alteration. In certain embodiments, the patient is resistant to treatment with erdafitinib or has acquired resistance to treatment with erdafitinib prior to step (a) of evaluating a biological sample from the patient for the presence of at least one BRAF, EGFR, CCND1, ARID1A, ErbB2 or TERT genetic alteration

In certain embodiments, the patient received at least one systemic therapy for the treatment of cancer generally, and mUC more specifically, prior to administration of any of the combination therapies described herein. In some embodiments, the at least one systemic therapy for the treatment of cancer generally, and mUC more specifically, is platinum-containing chemotherapy. In further embodiments, the cancer generally, and mUC more specifically, progressed during or following at least one line of the platinum-containing chemotherapy.

### Certain Nucleotide and Amino Acid Sequences

The nucleotide sequences for the FGFR fusion cDNA are provided in Table 4. The underlined sequences correspond to either FGFR3 or FGFR2, the sequences in black represent the fusion partners.

TABLE 4

| | |
|---|---|
| FGFR3-TACC3 V1 | >ATGGGCGCCCCTGCCTGCGCCCTCGCGCTCTGCGTGGCCGTGGCCATCGT |
| (2850 base pairs) | GGCCGGCGCCTCCTCGGAGTCCTTGGGGACGGAGCAGCGCGTCGTGGGGC |
| (SEQ ID NO: 33) | GAGCGGCAGAAGTCCCGGGCCCAGAGCCCGGCCAGCAGGAGCAGTTGGT |
| | CTTCGGCAGCGGGGATGCTGTGGAGCTGAGCTGTCCCCCGCCCGGGGGTG |
| | GTCCCATGGGGCCCACTGTCTGGGTCAAGGATGGCACAGGGCTGGTGCCC |
| | TCGGAGCGTGTCCTGGTGGGGCCCCAGCGGCTGCAGGTGCTGAATGCCTC |
| | CCACGAGGACTCCGGGGCCTACAGCTGCCGGCAGCGGCTCACGCAGCGC |
| | GTACTGTGCCACTTCAGTGTGCGGGTGACAGACGCTCCATCCTCGGGAGA |
| | TGACGAAGACGGGGAGGACGAGGCTGAGGACACAGGTGTGGACACAGG |
| | GGCCCCTTACTGGACACGGCCCGAGCGGATGGACAAGAAGCTGCTGGCC |
| | GTGCCGGCCGCCAACACCGTCCGCTTCCGCTGCCCAGCCGCTGGCAACCC |
| | CACTCCCTCCATCTCCTGGCTGAAGAACGGCAGGGAGTTCCGCGGCGAGC |
| | ACCGCATTGGAGGCATCAAGCTGCGGCATCAGCAGTGGAGCCTGGTCATG |
| | GAAAGCGTGGTGCCCTCGGACCGCGGCAACTACACCTGCGTCGTGGAGA |
| | ACAAGTTTGGCAGCATCCGGCAGACGTACACGCTGGACGTGCTGGAGCGC |
| | TCCCCGCACCGGCCCATCCTGCAGGCGGGGCTGCCGGCCAACCAGACGGC |
| | GGTGCTGGGCAGCGACGTGGAGTTCCACTGCAAGGTGTACAGTGACGCAC |
| | AGCCCCACATCCAGTGGCTCAAGCACGTGGAGGTGAATGGCAGCAAGGT |
| | GGGCCCGGACGGCACACCCTACGTTACCGTGCTCAAGACGGCGGGCGCTA |
| | ACACCACCGACAAGGAGCTAGAGGTTCTCTCCTTGCACAACGTCACCTTT |
| | GAGGACGCCGGGGAGTACACCTGCCTGGCGGGCAATTCTATTGGGTTTTC |
| | TCATCACTCTGCGTGGCTGGTGGTGCTGCCAGCCGAGGAGGAGCTGGTGG |
| | AGGCTGACGAGGCGGGCAGTGTGTATGCAGGCATCCTCAGCTACGGGGT |
| | GGGCTTCTTCCTGTTCATCCTGGTGGTGGCGGCTGTGACGCTCTGCCGCCT |
| | GCGCAGCCCCCCCAAGAAAGGCCTGGGCTCCCCCACCGTGCACAAGATCT |
| | CCCGCTTCCCGCTCAAGCGACAGGTGTCCCTGGAGTCCAACGCGTCCATG |
| | AGCTCCAACACACCACTGGTGCGCATCGCAAGGCTGTCCTCAGGGGAGGG |
| | CCCCACGCTGGCCAATGTCTCCGAGCTCGAGCTGCCTGCCGACCCCAAAT |
| | GGGAGCTGTCTCGGGCCCGGCTGACCCTGGGCAAGCCCCTTGGGGAGGGC |
| | TGCTTCGGCCAGGTGGTCATGGCGGAGGCCATCGGCATTGACAAGGACCG |
| | GGCCGCCAAGCCTGTCACCGTAGCCGTGAAGATGCTGAAAGACGATGCC |
| | ACTGACAAGGACCTGTCGGACCTGGTGTCTGAGATGGAGATGATGAAGAT |
| | GATCGGGAAACACAAAAACATCATCAACCTGCTGGGCGCCTGCACGCAG |
| | GGCGGGCCCCTGTACGTGCTGGTGGAGTACGCGGCCAAGGGTAACCTGCG |
| | GGAGTTTCTGCGGGCGCGGCGGCCCCCGGGCCTGGACTACTCCTTCGACA |
| | CCTGCAAGCCGCCCGAGGAGCAGCTCACCTTCAAGGACCTGGTGTCCTGT |
| | GCCTACCAGGTGGCCCGGGGCATGGAGTACTTGGCCTCCCAGAAGTGCAT |
| | CCACAGGGACCTGGCTGCCCGCAATGTGCTGGTGACCGAGGACAACGTG |
| | ATGAAGATCGCAGACTTCGGGCTGGCCCGGGACGTGCACAACCTCGACTA |
| | CTACAAGAAGACGACCAACGGCCGGCTGCCCGTGAAGTGGATGGCGCCT |
| | GAGGCCTTGTTTGACCGAGTCTACACTCACCAGAGTGACGTCTGGTCCTTT |
| | GGGGTCCTGCTCTGGGAGATCTTCACGCTGGGGGGCTCCCCGTACCCCGG |
| | CATCCCTGTGGAGGAGCTCTTCAAGCTGCTGAAGGAGGGCCACCGCATGG |
| | ACAAGCCCGCCAACTGCACACACGACCTGTACATGATCATGCGGGAGTGC |
| | TGGCATGCCGCGCCCTCCCAGAGGCCCACCTTCAAGCAGCTGGTGGAGGA |
| | CCTGGACCGTGTCCTTACCGTGACGTCCACCGACGTAAAGGCGACACAGG |
| | AGGAGAACCGGGAGCTGAGGAGCAGGTGTGAGGAGCTCCACGGGAAGA |
| | ACCTGGAACTGGGGAAGATCATGGACAGGTTCGAAGAGGTTGTGTACCA |
| | GGCCATGGAGGAAGTTCAGAAGCAGAAGGAACTTTCCAAAGCTGAAATC |
| | CAGAAAGTTCTAAAAGAAAAAGACCAACTTACCACAGATCTGAACTCCAT |
| | GGAGAAGTCCTTCTCCGACCTCTTCAAGCGTTTTGAGAAACAGAAAGAGG |
| | TGATCGAGGGCTACCGCAAGAACGAAGAGTCACTGAAGAAGTGCGTGGA |
| | GGATTACCTGGCAAGGATCACCCAGGAGGGCCAGAGGTACCAAGCCCTG |
| | AAGGCCCACGCGGAGGAGAAGCTGCAGCTGGCAAACGAGGAGATCGCCC |
| | AGGTCCGGAGCAAGGCCCAGGCGGAAGCGTTGGCCCTCCAGGCCAGCCT |
| | GAGGAAGGAGCAGATGCGCATCCAGTCGCTGGAGAAGACAGTGGAGCAG |
| | AAGACTAAAGAGAACGAGGAGCTGACCAGGATCTGCGACGACCTCATCT |
| | CCAAGATGGAGAAGATCTGA |
| FGFR3-TACC3 V3 | >ATGGGCGCCCCTGCCTGCGCCCTCGCGCTCTGCGTGGCCGTGGCCATCGT |
| (2955 base pairs) | GGCCGGCGCCTCCTCGGAGTCCTTGGGGACGGAGCAGCGCGTCGTGGGGC |
| (SEQ ID NO: 34 | GAGCGGCAGAAGTCCCGGGCCCAGAGCCCGGCCAGCAGGAGCAGTTGGT |
| | CTTCGGCAGCGGGGATGCTGTGGAGCTGAGCTGTCCCCCGCCCGGGGGTG |
| | GTCCCATGGGGCCCACTGTCTGGGTCAAGGATGGCACAGGGCTGGTGCCC |
| | TCGGAGCGTGTCCTGGTGGGGCCCCAGCGGCTGCAGGTGCTGAATGCCTC |
| | CCACGAGGACTCCGGGGCCTACAGCTGCCGGCAGCGGCTCACGCAGCGC |
| | GTACTGTGCCACTTCAGTGTGCGGGTGACAGACGCTCCATCCTCGGGAGA |
| | TGACGAAGACGGGGAGGACGAGGCTGAGGACACAGGTGTGGACACAGG |
| | GGCCCCTTACTGGACACGGCCCGAGCGGATGGACAAGAAGCTGCTGGCC |
| | GTGCCGGCCGCCAACACCGTCCGCTTCCGCTGCCCAGCCGCTGGCAACCC |
| | CACTCCCTCCATCTCCTGGCTGAAGAACGGCAGGGAGTTCCGCGGCGAGC |
| | ACCGCATTGGAGGCATCAAGCTGCGGCATCAGCAGTGGAGCCTGGTCATG |
| | GAAAGCGTGGTGCCCTCGGACCGCGGCAACTACACCTGCGTCGTGGAGA |
| | ACAAGTTTGGCAGCATCCGGCAGACGTACACGCTGGACGTGCTGGAGCGC |
| | TCCCCGCACCGGCCCATCCTGCAGGCGGGGCTGCCGGCCAACCAGACGGC |
| | GGTGCTGGGCAGCGACGTGGAGTTCCACTGCAAGGTGTACAGTGACGCAC |
| | AGCCCCACATCCAGTGGCTCAAGCACGTGGAGGTGAATGGCAGCAAGGT |
| | GGGCCCGGACGGCACACCCTACGTTACCGTGCTCAAGACGGCGGGCGCTA |
| | ACACCACCGACAAGGAGCTAGAGGTTCTCTCCTTGCACAACGTCACCTTT |
| | GAGGACGCCGGGGAGTACACCTGCCTGGCGGGCAATTCTATTGGGTTTTC |
| | TCATCACTCTGCGTGGCTGGTGGTGCTGCCAGCCGAGGAGGAGCTGGTGG |
| | AGGCTGACGAGGCGGGCAGTGTGTATGCAGGCATCCTCAGCTACGGGGT |
| | GGGCTTCTTCCTGTTCATCCTGGTGGTGGCGGCTGTGACGCTCTGCCGCCT |
| | GCGCAGCCCCCCCAAGAAAGGCCTGGGCTCCCCCACCGTGCACAAGATCT |
| | CCCGCTTCCCGCTCAAGCGACAGGTGTCCCTGGAGTCCAACGCGTCCATG |
| | AGCTCCAACACACCACTGGTGCGCATCGCAAGGCTGTCCTCAGGGGAGGG |
| | CCCCACGCTGGCCAATGTCTCCGAGCTCGAGCTGCCTGCCGACCCCAAAT |
| | GGGAGCTGTCTCGGGCCCGGCTGACCCTGGGCAAGCCCCTTGGGGAGGGC |
| | TGCTTCGGCCAGGTGGTCATGGCGGAGGCCATCGGCATTGACAAGGACCG |
| | GGCCGCCAAGCCTGTCACCGTAGCCGTGAAGATGCTGAAAGACGATGCC |
| | ACTGACAAGGACCTGTCGGACCTGGTGTCTGAGATGGAGATGATGAAGAT |
| | GATCGGGAAACACAAAAACATCATCAACCTGCTGGGCGCCTGCACGCAG |
| | GGCGGGCCCCTGTACGTGCTGGTGGAGTACGCGGCCAAGGGTAACCTGCG |
| | GGAGTTTCTGCGGGCGCGGCGGCCCCCGGGCCTGGACTACTCCTTCGACA |
| | CCTGCAAGCCGCCCGAGGAGCAGCTCACCTTCAAGGACCTGGTGTCCTGT |
| | GCCTACCAGGTGGCCCGGGGCATGGAGTACTTGGCCTCCCAGAAGTGCAT |
| | CCACAGGGACCTGGCTGCCCGCAATGTGCTGGTGACCGAGGACAACGTG |
| | ATGAAGATCGCAGACTTCGGGCTGGCCCGGGACGTGCACAACCTCGACTA |
| | CTACAAGAAGACGACCAACGGCCGGCTGCCCGTGAAGTGGATGGCGCCT |
| | GAGGCCTTGTTTGACCGAGTCTACACTCACCAGAGTGACGTCTGGTCCTTT |
| | GGGGTCCTGCTCTGGGAGATCTTCACGCTGGGGGGCTCCCCGTACCCCGG |
| | CATCCCTGTGGAGGAGCTCTTCAAGCTGCTGAAGGAGGGCCACCGCATGG |
| | ACAAGCCCGCCAACTGCACACACGACCTGTACATGATCATGCGGGAGTGC |
| | TGGCATGCCGCGCCCTCCCAGAGGCCCACCTTCAAGCAGCTGGTGGAGGA |
| | CCTGGACCGTGTCCTTACCGTGACGTCCACCGACGTGCCAGGCCCACCCC |
| | CAGGTGTTCCCGCGCCTGGGGGCCCACCCCTGTCCACCGGACCTATAGTG |
| | GACCTGCTCCAGTACAGCCAGAAGGACCTGGATGCAGTGGTAAAGGCGA |
| | CACAGGAGGAGAACCGGGAGCTGAGGAGCAGGTGTGAGGAGCTCCACGG |
| | GAAGAACCTGGAACTGGGGAAGATCATGGACAGGTTCGAAGAGGTTGTG |
| | TACCAGGCCATGGAGGAAGTTCAGAAGCAGAAGGAACTTTCCAAAGCTG |
| | AAATCCAGAAAGTTCTAAAAGAAAAAGACCAACTTACCACAGATCTGAA |
| | CTCCATGGAGAAGTCCTTCTCCGACCTCTTCAAGCGTTTTGAGAAACAGA |
| | AAGAGGTGATCGAGGGCTACCGCAAGAACGAAGAGTCACTGAAGAAGTG |
| | CGTGGAGGATTACCTGGCAAGGATCACCCAGGAGGGCCAGAGGTACCAA |
| | GCCCTGAAGGCCCACGCGGAGGAGAAGCTGCAGCTGGCAAACGAGGAGA |
| | TCGCCCAGGTCCGGAGCAAGGCCCAGGCGGAAGCGTTGGCCCTCCAGGC |
| | CAGCCTGAGGAAGGAGCAGATGCGCATCCAGTCGCTGGAGAAGACAGTG |
| | GAGCAGAAGACTAAAGAGAACGAGGAGCTGACCAGGATCTGCGACGACC |
| | TCATCTCCAAGATGGAGAAGATCTGA |
| FGFR3-BAIAP2L1 | >ATGGGCGCCCCTGCCTGCGCCCTCGCGCTCTGCGTGGCCGTGGCCATCGT |
| (3765 base pairs) | GGCCGGCGCCTCCTCGGAGTCCTTGGGGACGGAGCAGCGCGTCGTGGGGC |
| (SEQ ID NO: 35) | GAGCGGCAGAAGTCCCGGGCCCAGAGCCCGGCCAGCAGGAGCAGTTGGT |
| | CTTCGGCAGCGGGGATGCTGTGGAGCTGAGCTGTCCCCCGCCCGGGGGTG |
| | GTCCCATGGGGCCCACTGTCTGGGTCAAGGATGGCACAGGGCTGGTGCCC |
| | TCGGAGCGTGTCCTGGTGGGGCCCCAGCGGCTGCAGGTGCTGAATGCCTC |
| | CCACGAGGACTCCGGGGCCTACAGCTGCCGGCAGCGGCTCACGCAGCGC |
| | GTACTGTGCCACTTCAGTGTGCGGGTGACAGACGCTCCATCCTCGGGAGA |
| | TGACGAAGACGGGGAGGACGAGGCTGAGGACACAGGTGTGGACACAGG |
| | GGCCCCTTACTGGACACGGCCCGAGCGGATGGACAAGAAGCTGCTGGCC |
| | GTGCCGGCCGCCAACACCGTCCGCTTCCGCTGCCCAGCCGCTGGCAACCC |
| | CACTCCCTCCATCTCCTGGCTGAAGAACGGCAGGGAGTTCCGCGGCGAGC |
| | ACCGCATTGGAGGCATCAAGCTGCGGCATCAGCAGTGGAGCCTGGTCATG |
| | GAAAGCGTGGTGCCCTCGGACCGCGGCAACTACACCTGCGTCGTGGAGA |
| | ACAAGTTTGGCAGCATCCGGCAGACGTACACGCTGGACGTGCTGGAGCGC |
| | TCCCCGCACCGGCCCATCCTGCAGGCGGGGCTGCCGGCCAACCAGACGGC |
| | GGTGCTGGGCAGCGACGTGGAGTTCCACTGCAAGGTGTACAGTGACGCAC |
| | AGCCCCACATCCAGTGGCTCAAGCACGTGGAGGTGAATGGCAGCAAGGT |
| | GGGCCCGGACGGCACACCCTACGTTACCGTGCTCAAGTCCTGGATCAGTG |
| | AGAGTGTGGAGGCCGACGTGCGCCTCCGCCTGGCCAATGTGTCGGAGCGG |
| | GACGGGGGCGAGTACCTCTGTCGAGCCACCAATTTCATAGGCGTGGCCGA |
| | GAAGGCCTTTTGGCTGAGCGTTCACGGGCCCCGAGCAGCCGAGGAGGAG |
| | CTGGTGGAGGCTGACGAGGCGGGCAGTGTGTATGCAGGCATCCTCAGCTA |
| | CGGGGTGGGCTTCTTCCTGTTCATCCTGGTGGTGGCGGCTGTGACGCTCTG |
| | CCGCCTGCGCAGCCCCCCCAAGAAAGGCCTGGGCTCCCCCACCGTGCACA |
| | AGATCTCCCGCTTCCCGCTCAAGCGACAGGTGTCCCTGGAGTCCAACGCG |
| | TCCATGAGCTCCAACACACCACTGGTGCGCATCGCAAGGCTGTCCTCAGG |
| | GGAGGGCCCCACGCTGGCCAATGTCTCCGAGCTCGAGCTGCCTGCCGACC |
| | CCAAATGGGAGCTGTCTCGGGCCCGGCTGACCCTGGGCAAGCCCCTTGGG |
| | GAGGGCTGCTTCGGCCAGGTGGTCATGGCGGAGGCCATCGGCATTGACAA |
| | GGACCGGGCCGCCAAGCCTGTCACCGTAGCCGTGAAGATGCTGAAAGAC |
| | GATGCCACTGACAAGGACCTGTCGGACCTGGTGTCTGAGATGGAGATGAT |
| | GAAGATGATCGGGAAACACAAAAACATCATCAACCTGCTGGGCGCCTGC |
| | ACGCAGGGCGGGCCCCTGTACGTGCTGGTGGAGTACGCGGCCAAGGGTA |
| | ACCTGCGGGAGTTTCTGCGGGCGCGGCGGCCCCCGGGCCTGGACTACTCC |
| | TTCGACACCTGCAAGCCGCCCGAGGAGCAGCTCACCTTCAAGGACCTGGT |
| | GTCCTGTGCCTACCAGGTGGCCCGGGGCATGGAGTACTTGGCCTCCCAGA |
| | AGTGCATCCACAGGGACCTGGCTGCCCGCAATGTGCTGGTGACCGAGGAC |
| | AACGTGATGAAGATCGCAGACTTCGGGCTGGCCCGGGACGTGCACAACCT |
| | CGACTACTACAAGAAGACGACCAACGGCCGGCTGCCCGTGAAGTGGATG |
| | GCGCCTGAGGCCTTGTTTGACCGAGTCTACACTCACCAGAGTGACGTCTG |
| | GTCCTTTGGGGTCCTGCTCTGGGAGATCTTCACGCTGGGGGGCTCCCCGTA |
| | CCCCGGCATCCCTGTGGAGGAGCTCTTCAAGCTGCTGAAGGAGGGCCACC |
| | GCATGGACAAGCCCGCCAACTGCACACACGACCTGTACATGATCATGCGG |
| | GAGTGCTGGCATGCCGCGCCCTCCCAGAGGCCCACCTTCAAGCAGCTGGT |
| | GGAGGACCTGGACCGTGTCCTTACCGTGACGTCCACCGACAATGTTATGG |
| | AACAGTTCAATCCTGGGCTGCGAAATTTAATAAACCTGGGGAAAAATTAT |
| | GAGAAAGCTGTAAACGCTATGATCCTGGCAGGAAAAGCCTACTACGATG |
| | GAGTGGCCAAGATCGGTGAGATTGCCACTGGGTCCCCCGTGTCAACTGAA |
| | CTGGGACATGTCCTCATAGAGATTTCAAGTACCCACAAGAAACTCAACGA |
| | GAGTCTTGATGAAAATTTTAAAAAATTCCACAAAGAGATTATCCATGAGC |
| | TGGAGAAGAAGATAGAACTTGACGTGAAATATATGAACGCAACTCTAAA |
| | AAGATACCAAACAGAACACAAGAATAAATTAGAGTCTTTGGAGAAATCC |
| | CAAGCTGAGTTGAAGAAGATCAGAAGGAAAAGCCAAGGAAGCCGAAAC |
| | GCACTCAAATATGAACACAAAGAAATTGAGTATGTGGAGACCGTTACTTC |
| | TCGTCAGAGTGAAATCCAGAAATTCATTGCAGATGGTTGCAAAGAGGCTC |
| | TGCTTGAAGAGAAGAGGCGCTTCTGCTTTCTGGTTGATAAGCACTGTGGC |
| | TTTGCAAACCACATACATTATTATCACTTACAGTCTGCAGAACTACTGAAT |
| | TCCAAGCTGCCTCGGTGGCAGGAGACCTGTGTTGATGCCATCAAAGTGCC |
| | AGAGAAAATCATGAATATGATCGAAGAAATAAAGACCCCAGCCTCTACC |
| | CCCGTGTCTGGAACTCCTCAGGCTTCACCCATGATCGAGAGAAGCAATGT |
| | GGTTAGGAAAGATTACGACACCCTTTCTAAATGCTCACCAAAGATGCCCC |
| | CCGCTCCTTCAGGCAGAGCATATACCAGTCCCTTGATCGATATGTTTAATA |
| | ACCCAGCCACGGCTGCCCCGAATTCACAAAGGGTAAATAATTCAACAGGT |
| | ACTTCCGAAGATCCCAGTTTACAGCGATCAGTTTCGGTTGCAACGGGACT |
| | GAACATGATGAAGAAGCAGAAAGTGAAGACCATCTTCCCGCACACTGCG |
| | GGCTCCAACAAGACCTTACTCAGCTTTGCACAGGGAGATGTCATCACGCT |
| | GCTCATCCCCGAGGAGAAGGATGGCTGGCTCTATGGAGAACACGACGTGT |
| | CCAAGGCGAGGGGTTGGTTCCCGTCGTCGTACACGAAGTTGCTGGAAGAA |
| | AATGAGACAGAAGCAGTGACCGTGCCCACGCCAAGCCCCACACCAGTGA |
| | GAAGCATCAGCACCGTGAACTTGTCTGAGAATAGCAGTGTTGTCATCCCC |
| | CCACCCGACTACTTGGAATGCTTGTCCATGGGGGCAGCTGCCGACAGGAG |
| | AGCAGATTCGGCCAGGACGACATCCACCTTTAAGGCCCCAGCGTCCAAGC |
| | CCGAGACCGCGGCTCCTAACGATGCCAACGGGACTGCAAAGCCGCCTTTT |
| | CTCAGCGGAGAAAACCCCTTTGCCACTGTGAAACTCCGCCCGACTGTGAC |
| | GAATGATCGCTCGGCACCCATCATTCGATGA |
| FGFR2-BICC1 | >ATGGTCAGCTGGGGTCGTTTCATCTGCCTGGTCGTGGTCACCATGGCAAC |
| (4989 base pairs) | CTTGTCCCTGGCCCGGCCCTCCTTCAGTTTAGTTGAGGATACCACATTAGA |
| (SEQ ID NO: 36) | GCCAGAAGAGCCACCAACCAAATACCAAATCTCTCAACCAGAAGTGTAC |
| | GTGGCTGCGCCAGGGGAGTCGCTAGAGGTGCGCTGCCTGTTGAAAGATGC |
| | CGCCGTGATCAGTTGGACTAAGGATGGGGTGCACTTGGGGCCCAACAATA |
| | GGACAGTGCTTATTGGGGAGTACTTGCAGATAAAGGGCGCCACGCCTAGA |
| | GACTCCGGCCTCTATGCTTGTACTGCCAGTAGGACTGTAGACAGTGAAAC |
| | TTGGTACTTCATGGTGAATGTCACAGATGCCATCTCATCCGGAGATGATG |
| | AGGATGACACCGATGGTGCGGAAGATTTTGTCAGTGAGAACAGTAACAA |
| | CAAGAGAGCACCATACTGGACCAACACAGAAAAGATGGAAAAGCGGCTC |
| | CATGCTGTGCCTGCGGCCAACACTGTCAAGTTTCGCTGCCCAGCCGGGGG |
| | GAACCCAATGCCAACCATGCGGTGGCTGAAAAACGGGAAGGAGTTTAAG |
| | CAGGAGCATCGCATTGGAGGCTACAAGGTACGAAACCAGCACTGGAGCC |
| | TCATTATGGAAAGTGTGGTCCCATCTGACAAGGGAAATTATACCTGTGTA |
| | GTGGAGAATGAATACGGGTCCATCAATCACACGTACCACCTGGATGTTGT |
| | GGAGCGATCGCCTCACCGGCCCATCCTCCAAGCCGGACTGCCGGCAAATG |
| | CCTCCACAGTGGTCGGAGGAGACGTAGAGTTTGTCTGCAAGGTTTACAGT |
| | GATGCCCAGCCCCACATCCAGTGGATCAAGCACGTGGAAAAGAACGGCA |
| | GTAAATACGGGCCCGACGGGCTGCCCTACCTCAAGGTTCTCAAGGCCGCC |
| | GGTGTTAACACCACGGACAAAGAGATTGAGGTTCTCTATATTCGGAATGT |
| | AACTTTTGAGGACGCTGGGGAATATACGTGCTTGGCGGGTAATTCTATTG |
| | GGATATCCTTTCACTCTGCATGGTTGACAGTTCTGCCAGCGCCTGGAAGA |
| | GAAAAGGAGATTACAGCTTCCCCAGACTACCTGGAGATAGCCATTTACTG |
| | CATAGGGGTCTTCTTAATCGCCTGTATGGTGGTAACAGTCATCCTGTGCCG |
| | AATGAAGAACACGACCAAGAAGCCAGACTTCAGCAGCCAGCCGGCTGTG |
| | CACAAGCTGACCAAACGTATCCCCCTGCGGAGACAGGTAACAGTTTCGGC |
| | TGAGTCCAGCTCCTCCATGAACTCCAACACCCCGCTGGTGAGGATAACAA |
| | CACGCCTCTCTTCAACGGCAGACACCCCCATGCTGGCAGGGGTCTCCGAG |
| | TATGAACTTCCAGAGGACCCAAAATGGGAGTTTCCAAGAGATAAGCTGAC |
| | ACTGGGCAAGCCCCTGGGAGAAGGTTGCTTTGGGCAAGTGGTCATGGCGG |
| | AAGCAGTGGGAATTGACAAAGACAAGCCCAAGGAGGCGGTCACCGTGGC |
| | CGTGAAGATGTTGAAAGATGATGCCACAGAGAAAGACCTTTCTGATCTGG |
| | TGTCAGAGATGGAGATGATGAAGATGATTGGGAAACACAAGAATATCAT |
| | AAATCTTCTTGGAGCCTGCACACAGGATGGGCCTCTCTATGTCATAGTTG |
| | AGTATGCCTCTAAAGGCAACCTCCGAGAATACCTCCGAGCCCGGAGGCCA |
| | CCCGGGATGGAGTACTCCTATGACATTAACCGTGTTCCTGAGGAGCAGAT |
| | GACCTTCAAGGACTTGGTGTCATGCACCTACCAGCTGGCCAGAGGCATGG |
| | AGTACTTGGCTTCCCAAAAATGTATTCATCGAGATTTAGCAGCCAGAAAT |
| | GTTTTGGTAACAGAAAACAATGTGATGAAAATAGCAGACTTTGGACTCGC |
| | CAGAGATATCAACAATATAGACTATTACAAAAAGACCACCAATGGGCGG |
| | CTTCCAGTCAAGTGGATGGCTCCAGAAGCCCTGTTTGATAGAGTATACAC |
| | TCATCAGAGTGATGTCTGGTCCTTCGGGGTGTTAATGTGGGAGATCTTCAC |
| | TTTAGGGGGCTCGCCCTACCCAGGGATTCCCGTGGAGGAACTTTTTAAGC |
| | TGCTGAAGGAAGGACACAGAATGGATAAGCCAGCCAACTGCACCAACGA |
| | ACTGTACATGATGATGAGGGACTGTTGGCATGCAGTGCCCTCCCAGAGAC |
| | CAACGTTCAAGCAGTTGGTAGAAGACTTGGATCGAATTCTCACTCTCACA |
| | ACCAATGAGATCATGGAGGAAACAAATACGCAGATTGCTTGGCCATCAA |
| | AACTGAAGATCGGAGCCAAATCCAAGAAAGATCCCCATATTAAGGTTTCT |
| | GGAAAGAAAGAAGATGTTAAAGAAGCCAAGGAAATGATCATGTCTGTCT |
| | TAGACACAAAAAGCAATCGAGTCACACTGAAGATGGATGTTTCACATACA |
| | GAACATTCACATGTAATCGGCAAAGGTGGCAACAATATTAAAAAAGTGA |
| | TGGAAGAAACCGGATGCCATATCCACTTTCCAGATTCCAACAGGAATAAC |
| | CAAGCAGAAAAAAGCAACCAGGTATCTATAGCGGGACAACCAGCAGGAG |
| | TAGAATCTGCCCGAGTTAGAATTCGGGAGCTGCTTCCTTTGGTGCTGATGT |
| | TTGAGCTACCAATTGCTGGAATTCTTCAACCGGTTCCTGATCCTAATTCCC |
| | CCTCTATTCAGCATATATCACAAACGTACAATATTTCAGTATCATTTAAAC |
| | AGCGTTCCCGAATGTATGGTGCTACTGTCATAGTACGAGGGTCTCAGAAT |
| | AACACTAGTGCTGTGAAGGAAGGAACTGCCATGCTGTTAGAACATCTTGC |
| | TGGGAGCTTAGCATCAGCTATTCCTGTGAGCACACAACTAGATATTGCAG |
| | CTCAACATCATCTCTTTATGATGGGTCGAAATGGGAGCAACATCAAACAT |
| | ATCATGCAGAGAACAGGTGCTCAGATCCACTTTCCTGATCCCAGTAATCC |
| | ACAAAAGAAATCTACCGTCTACCTCCAGGGCACCATTGAGTCTGTCTGTC |
| | TTGCAAGGCAATATCTCATGGGTTGTCTTCCTCTTGTGTTGATGTTTGATA |
| | TGAAGGAAGAAATTGAAGTAGATCCACAATTCATTGCGCAGTTGATGGAA |
| | CAGCTTGATGTCTTCATCAGTATTAAACCAAAGCCCAAACAGCCAAGCAA |
| | GTCTGTGATTGTGAAAAGTGTTGAGCGAAATGCCTTAAATATGTATGAAG |
| | CAAGGAAATGTCTCCTCGGACTTGAAAGCAGTGGGGTTACCATAGCAACC |
| | AGTCCATCCCCAGCATCCTGCCCTGCCGGCCTGGCATGTCCCAGCCTGGA |
| | TATCTTAGCTTCAGCAGGCCTTGGACTCACTGGACTAGGTCTTTTGGGACC |
| | CACCACCTTATCTCTGAACACTTCAACAACCCCAAACTCACTCTTGAATGC |
| | TCTTAATAGCTCAGTCAGTCCTTTGCAAAGTCCAAGTTCTGGTACACCCAG |
| | CCCCACATTATGGGCACCCCCACTTGCTAATACTTCAAGTGCCACAGGTTT |
| | TTCTGCTATACCACACCTTATGATTCCATCTACTGCCCAAGCCACATTAAC |
| | TAATATTTTGTTGTCTGGAGTGCCCACCTATGGGCACACAGCTCCATCTCC |
| | CCCTCCTGGCTTGACTCCTGTTGATGTCCATATCAACAGTATGCAGACCGA |
| | AGGCAAAAAAATCTCTGCTGCTTTAAATGGACATGCACAGTCTCCAGATA |
| | TAAAATATGGTGCAATATCCACTTCATCACTTGGAGAAAAAGTGCTGAGT |
| | GCAAATCACGGGGATCCGTCCATCCAGACAAGTGGGTCTGAGCAGACATC |
| | TCCCAAATCAAGCCCCACTGAAGGTTGTAATGATGCTTTTGTTGAAGTAG |
| | GCATGCCTCGAAGTCCTTCCCATTCTGGGAATGCTGGTGACTTGAAACAG |
| | ATGATGTGTCCCTCCAAGGTTTCCTGTGCCAAAAGGCAGACAGTGGAACT |
| | ATTGCAAGGCACGAAAAACTCACACTTACACAGCACTGACAGGTTGCTCT |
| | CAGACCCTGAACTGAGTGCTACCGAAAGCCCTTTGGCTGACAAGAAGGCT |
| | CCAGGGAGTGAGCGCGCTGCAGAGAGGGCAGCAGCTGCCCAGCAAAACT |
| | CCGAAAGGGCCCACCTTGCTCCACGGTCATCATATGTCAACATGCAGGCA |
| | TTTGACTATGAACAGAAGAAGCTATTAGCCACCAAAGCTATGTTAAAGAA |
| | ACCAGTGGTGACGGAGGTCAGAACGCCCACAAATACCTGGAGTGGCCTG |
| | GGTTTTTCTAAATCCATGCCAGCTGAAACTATCAAGGAGTTGAGAAGGGC |
| | CAATCATGTGTCCTATAAGCCCACAATGACAACCACTTATGAGGGCTCAT |
| | CCATGTCCCTTTCACGGTCCAACAGTCGTGAGCACTTGGGAGGTGGAAGC |
| | GAATCTGATAACTGGAGAGACCGAAATGGAATTGGACCTGGAAGTCATA |
| | GTGAATTTGCAGCTTCTATTGGCAGCCCTAAGCGTAAACAAAACAAATCA |
| | ACGGAACACTATCTCAGCAGTAGCAATTACATGGACTGCATTTCCTCGCT |
| | GACAGGAAGCAATGGCTGTAACTTAAATAGCTCTTTCAAAGGTTCTGACC |
| | TCCCTGAGCTCTTCAGCAAACTGGGCCTGGGCAAATACACAGATGTTTTC |
| | CAGCAACAAGAGATCGATCTTCAGACATTCCTCACTCTCACAGATCAGGA |
| | TCTGAAGGAGCTGGGAATAACTACTTTTGGTGCCAGGAGGAAAATGCTGC |
| | TTGCAATTTCAGAACTAAATAAAAACCGAAGAAAGCTTTTTGAATCGCCA |
| | AATGCACGCACCTCTTTCCTGGAAGGTGGAGCGAGTGGAAGGCTACCCCG |
| | TCAGTATCACTCAGACATTGCTAGTGTCAGTGGCCGCTGGTAG |
| FGFR2-CASP7 | >ATGGTCAGCTGGGGTCGTTTCATCTGCCTGGTCGTGGTCACCATGGCAAC |
| (3213 base pairs) | CTTGTCCCTGGCCCGGCCCTCCTTCAGTTTAGTTGAGGATACCACATTAGA |
| (SEQ ID NO: 37) | GCCAGAAGAGCCACCAACCAAATACCAAATCTCTCAACCAGAAGTGTAC |
| | GTGGCTGCGCCAGGGGAGTCGCTAGAGGTGCGCTGCCTGTTGAAAGATGC |
| | CGCCGTGATCAGTTGGACTAAGGATGGGGTGCACTTGGGGCCCAACAATA |
| | GGACAGTGCTTATTGGGGAGTACTTGCAGATAAAGGGCGCCACGCCTAGA |
| | GACTCCGGCCTCTATGCTTGTACTGCCAGTAGGACTGTAGACAGTGAAAC |
| | TTGGTACTTCATGGTGAATGTCACAGATGCCATCTCATCCGGAGATGATG |
| | AGGATGACACCGATGGTGCGGAAGATTTTGTCAGTGAGAACAGTAACAA |
| | CAAGAGAGCACCATACTGGACCAACACAGAAAAGATGGAAAAGCGGCTC |
| | CATGCTGTGCCTGCGGCCAACACTGTCAAGTTTCGCTGCCCAGCCGGGGG |
| | GAACCCAATGCCAACCATGCGGTGGCTGAAAAACGGGAAGGAGTTTAAG |
| | CAGGAGCATCGCATTGGAGGCTACAAGGTACGAAACCAGCACTGGAGCC |
| | TCATTATGGAAAGTGTGGTCCCATCTGACAAGGGAAATTATACCTGTGTA |
| | GTGGAGAATGAATACGGGTCCATCAATCACACGTACCACCTGGATGTTGT |
| | GGAGCGATCGCCTCACCGGCCCATCCTCCAAGCCGGACTGCCGGCAAATG |
| | CCTCCACAGTGGTCGGAGGAGACGTAGAGTTTGTCTGCAAGGTTTACAGT |
| | GATGCCCAGCCCCACATCCAGTGGATCAAGCACGTGGAAAAGAACGGCA |
| | GTAAATACGGGCCCGACGGGCTGCCCTACCTCAAGGTTCTCAAGGCCGCC |
| | GGTGTTAACACCACGGACAAAGAGATTGAGGTTCTCTATATTCGGAATGT |
| | AACTTTTGAGGACGCTGGGGAATATACGTGCTTGGCGGGTAATTCTATTG |
| | GGATATCCTTTCACTCTGCATGGTTGACAGTTCTGCCAGCGCCTGGAAGA |
| | GAAAAGGAGATTACAGCTTCCCCAGACTACCTGGAGATAGCCATTTACTG |
| | CATAGGGGTCTTCTTAATCGCCTGTATGGTGGTAACAGTCATCCTGTGCCG |
| | AATGAAGAACACGACCAAGAAGCCAGACTTCAGCAGCCAGCCGGCTGTG |
| | CACAAGCTGACCAAACGTATCCCCCTGCGGAGACAGGTAACAGTTTCGGC |
| | TGAGTCCAGCTCCTCCATGAACTCCAACACCCCGCTGGTGAGGATAACAA |
| | CACGCCTCTCTTCAACGGCAGACACCCCCATGCTGGCAGGGGTCTCCGAG |
| | TATGAACTTCCAGAGGACCCAAAATGGGAGTTTCCAAGAGATAAGCTGAC |
| | ACTGGGCAAGCCCCTGGGAGAAGGTTGCTTTGGGCAAGTGGTCATGGCGG |
| | AAGCAGTGGGAATTGACAAAGACAAGCCCAAGGAGGCGGTCACCGTGGC |
| | CGTGAAGATGTTGAAAGATGATGCCACAGAGAAAGACCTTTCTGATCTGG |
| | TGTCAGAGATGGAGATGATGAAGATGATTGGGAAACACAAGAATATCAT |
| | AAATCTTCTTGGAGCCTGCACACAGGATGGGCCTCTCTATGTCATAGTTG |
| | AGTATGCCTCTAAAGGCAACCTCCGAGAATACCTCCGAGCCCGGAGGCCA |
| | CCCGGGATGGAGTACTCCTATGACATTAACCGTGTTCCTGAGGAGCAGAT |
| | GACCTTCAAGGACTTGGTGTCATGCACCTACCAGCTGGCCAGAGGCATGG |
| | AGTACTTGGCTTCCCAAAAATGTATTCATCGAGATTTAGCAGCCAGAAAT |
| | GTTTTGGTAACAGAAAACAATGTGATGAAAATAGCAGACTTTGGACTCGC |
| | CAGAGATATCAACAATATAGACTATTACAAAAAGACCACCAATGGGCGG |
| | CTTCCAGTCAAGTGGATGGCTCCAGAAGCCCTGTTTGATAGAGTATACAC |
| | TCATCAGAGTGATGTCTGGTCCTTCGGGGTGTTAATGTGGGAGATCTTCAC |
| | TTTAGGGGGCTCGCCCTACCCAGGGATTCCCGTGGAGGAACTTTTTAAGC |
| | TGCTGAAGGAAGGACACAGAATGGATAAGCCAGCCAACTGCACCAACGA |
| | ACTGTACATGATGATGAGGGACTGTTGGCATGCAGTGCCCTCCCAGAGAC |
| | CAACGTTCAAGCAGTTGGTAGAAGACTTGGATCGAATTCTCACTCTCACA |
| | ACCAATGAGATGGCAGATGATCAGGGCTGTATTGAAGAGCAGGGGGTTG |
| | AGGATTCAGCAAATGAAGATTCAGTGGATGCTAAGCCAGACCGGTCCTCG |
| | TTTGTACCGTCCCTCTTCAGTAAGAAGAAGAAAAATGTCACCATGCGATC |
| | CATCAAGACCACCCGGGACCGAGTGCCTACATATCAGTACAACATGAATT |
| | TTGAAAAGCTGGGCAAATGCATCATAATAAACAACAAGAACTTTGATAA |
| | AGTGACAGGTATGGGCGTTCGAAACGGAACAGACAAAGATGCCGAGGCG |
| | CTCTTCAAGTGCTTCCGAAGCCTGGGTTTTGACGTGATTGTCTATAATGAC |
| | TGCTCTTGTGCCAAGATGCAAGATCTGCTTAAAAAAGCTTCTGAAGAGGA |
| | CCATACAAATGCCGCCTGCTTCGCCTGCATCCTCTTAAGCCATGGAGAAG |
| | AAAATGTAATTTATGGGAAAGATGGTGTCACACCAATAAAGGATTTGACA |
| | GCCCACTTTAGGGGGGATAGATGCAAAACCCTTTTAGAGAAACCCAAACT |
| | CTTCTTCATTCAGGCTTGCCGAGGGACCGAGCTTGATGATGGCATCCAGG |
| | CCGACTCGGGGCCCATCAATGACACAGATGCTAATCCTCGATACAAGATC |
| | CCAGTGGAAGCTGACTTCCTCTTCGCCTATTCCACGGTTCCAGGCTATTAC |
| | TCGTGGAGGAGCCCAGGAAGAGGCTCCTGGTTTGTGCAAGCCCTCTGCTC |
| | CATCCTGGAGGAGCACGGAAAAGACCTGGAAATCATGCAGATCCTCACC |
| | AGGGTGAATGACAGAGTTGCCAGGCACTTTGAGTCTCAGTCTGATGACCC |
| | ACACTTCCATGAGAAGAAGCAGATCCCCTGTGTGGTCTCCATGCTCACCA |
| | AGGAACTCTACTTCAGTCAATAG |

The nucleic acid sequences for the EGFR, EGF and c-Met proteins are provided in Table 5.

TABLE 5

| | | |
|---|---|---|
| PRT, Homo | SEQ ID NO: 38 | MRPSGTAGAALLALLAALCPASRALEEKKVCQGTS |
| Sapiens, EGFR | | NKLTQLGTFEDHFLSLQRMFNNCEVVLGNLEITYVQ |
| (includes signal | | RNYDLSFLKTIQEVAGYVLIALNTVERIPLENLQUIRG |
| sequence of 24 aa. | | NMYYENSYALAVLSNYDANKTGLKELPMRNLQEIL |
| Mature protein | | HGAVRFSNNPALCNVESIQWRDIVSSDFLSNMSMDF |
| starts at residue 25) | | QNHLGSCQKCDPSCPNGSCWGAGEENCQKLTKIIC |
| | | AQQCSGRCRGKSPSDCCHNQCAAGCTGPRESDCLV |
| | | CRKFRDEATCKDTCPPLMLYNPTTYQMDVNPEGKY |
| | | SFGATCVKKCPRNYVVTDHGSCVRACGADSYEME |
| | | EDGVRKCKKCEGPCRKVCNGIGIGEFKDSLSINATNI |
| | | KHFKNCTSISGDLHILPVAFRGDSFTHTPPLDPQELDI |
| | | LKTVKEITGFLLIQAWPENRTDLHAFENLEIIRGRTK |
| | | QHGQFSLAVVSLNITSLGLRSLKEISDGDVIISGNKN |
| | | LCYANTINWKKLFGTSGQKTKIISNRGENSCKATGQ |
| | | VCHALCSPEGCWGPEPRDCVSCRNVSRGRECVDKC |
| | | NLLEGEPREFVENSECIQCHPECLPQAMNITCTGRGP |
| | | DNCIQCAHYIDGPHCVKTCPAGVMGENNTLVWKY |
| | | ADAGHVCHLCHPNCTYGCTGPGLEGCPTNGPKIPSI |
| | | ATGMVGALLLLLVVALGIGLFMRRRHIVRKRTLRR |
| | | LLQERELVEPLTPSGEAPNQALLRILKETEFKKIKVL |
| | | GSGAFGTVYKGLWIPEGEKVKIPVAIKELREATSPK |
| | | ANKEILDEAYVMASVDNPHVCRLLGICLTSTVQLIT |
| | | QLMPFGCLLDYVREHKDNIGSQYLLNWCVQIAKG |
| | | MNYLEDRRLVHRDLAARNVLVKTPQHVKITDFGLA |
| | | KLLGAEEKEYHAEGGKVPIKWMALESILHRIYTHQS |
| | | DVWSYGVTVWELMTFGSKPYDGIPASEISSILEKGE |
| | | RLPQPPICTIDVYMIMVKCWMIDADSRPKFRELIIEFS |
| | | KMARDPQRYLVIQGDERMHLPSPTDSNFYRALMDE |
| | | EDMDDVVDADEYLIPQQGFFSSPSTSRTPLLSSLSAT |
| | | SNNSTVACIDRNGLQSCPIKEDSFLQRYSSDPTGALT |
| | | EDSIDDTFLPVPEYINQSVPKRPAGSVQNPVYHNQPL |
| | | NPAPSRDPHYQDPHSTAVGNPEYLNTVQPTCVNSTF |
| | | DSPAHWAQKGSHQISLDNPDYQQDFFPKEAKPNGIF |
| | | KGSTAENAEYLRVAPQSSEFIGA |
| PRT, Homo | SEQ ID NO: 39 | NSDSECPLSHDGYCLHDGVCMYIEALDKYACNCVV |
| sapiens, EGF | | GYIGERCQYRDLKWWELR |
| PRT Homo sapiens | SEQ ID NO: 40 | MKAPAVLAPGILVLLFTLVQRSNGECKEALAKSEM |
| cMet | | NVNMKYQLPNFTAETPIQNVILHEHHIFLGATNYIY |
| | | VLNEEDLQKVAEYKTGPVLEHPDCFPCQDCSSKAN |
| | | LSGGVWKDNINMALVVDTYYDDQLISCGSVNRGTC |
| | | QRHVFPHNHTADIQSEVHCIFSPQIEEPSQCPDCVVS |
| | | ALGAKVLSSVKDRFINFFVGNTINSSYFPDHPLHSIS |
| | | VRRLKETKDGFMFLTDQSYIDVLPEFRDSYPIKYVH |
| | | AFESNNFIYFLTVQRETLDAQTFHTRIIRFCSINSGLH |
| | | SYMEMPLECILTEKRKKRSTKKEVFNILQAAYVSKP |
| | | GAQLARQIGASLNDDILFGVFAQSKPDSAEPMDRSA |
| | | MCAFPIKYVNDFFNKIVNKNNVRCLQHFYGPNHEH |
| | | CFNRTLLRNSSGCEARRDEYRTEFTTALQRVDLFMG |
| | | QFSEVLLTSISTFIKGDLTIANLGTSEGRFMQVVVSR |
| | | SGPSTPHVNFLLDSHPVSPEVIVEHTLNQNGYTLVIT |
| | | GKKITKIPLNGLGCRHFQSCSQCLSAPPFVQCGWCH |
| | | DKCVRSEECLSGTWTQQICLPAIYKVFPNSAPLEGG |
| | | TRLTICGWDFGFRRNNKFDLKKTRVLLGNESCTLTL |
| | | SESTMNTLKCTVGPAMNKHFNMSIIISNGHGTTQYS |
| | | TFSYVDPVITSISPKYGPMAGGTLLTLTGNYLNSGNS |
| | | RHISIGGKTCTLKSVSNSILECYTPAQTISTEFAVKLK |
| | | IDLANRETSIFSYREDPIVYEIHPTKSFISTWWKEPLNI |
| | | VSFLFCFASGGSTITGVGKNLNSVSVPRMVINVHEA |
| | | GRNFTVACQHRSNSEIICCTTPSLQQLNLQLPLKTKA |
| | | FFMLDGILSKYFDLIYVHNPVFKPFEKPVMISMGNE |
| | | NVLEIKGNDIDPEAVKGEVLKVGNKSCENIHLHSEA |
| | | VLCTVPNDLLKLNSELNIEWKQAISSTVLGKVIVQP |
| | | DQNFTGLIAGVVSISTALLLLLGFFLWLKKRKQIKDL |
| | | GSELVRYDARVHTPHLDRLVSARSVSPTTEMVSNES |
| | | VDYRATFPEDQFPNSSQNGSCRQVQYPLTDMSPILT |
| | | SGDSDISSPLLQNTVHIDLSALNPELVQAVQHVVIGP |
| | | SSLIVHFNEVIGRGHFGCVYHGTLLDNDGKKIHCAV |
| | | KSLNRITDIGEVSQFLTEGIIMKDFSHPNVLSLLGICL |
| | | RSEGSPLVVLPYMKHGDLRNFIRNETHNPTVKDLIG |
| | | FGLQVAKGMKYLASKKFVHRDLAARNCMLDEKFT |
| | | VKVADFGLARDMYDKEYYSVHNKTGAKLPVKWM |
| | | ALESLQTQKFTTKSDVWSFGVLLWELMTRGAPPYP |
| | | DVNTFDITVYLLQGRRLLQPEYCPDPLYEVMLKCW |
| | | HPKAEMRPSFSELVSRISAIFSTFIGEHYVHVNATYV |
| | | NVKCVAPYPSLLSSEDNADDEVDTRPASFWETS |

The nucleic acid sequences for the heavy and light chains of an EGFR/c-Met bispecific antibody described herein are provided in Table 6.

TABLE 6

| | | |
|---|---|---|
| EM1-mAb H1 | SEQ ID NO: 41 | QVQLVESGGGVVQPGRSLRLSCAASGFTFSTYGMH |
| (anti-EGFR, 405L) | | WVRQAPGKGLEWVAVIWDDGSYKYYGDSVKGRF |
| | | TISRDNSKNTLYLQMNSLRAEDTAVYYCARDGITM |
| | | VRGVMKDYFDYWGQGTLVTVSSASTKGPSVFPLAP |
| | | SSKSTSGGTAALGCLVKDYFPEPVTVSWNSGALTSG |
| | | VHTFPAVLQSSGLYSLSSVVTVPSSSLGTQTYICNVN |
| | | HKPSNTKVDKRVEPKSCDKTHTCPPCPAPELLGGPS |
| | | VFLFPPKPKDTLMISRTPEVTCVVVDVSHEDPEVKF |
| | | NWYVDGVEVHNAKTKPREEQYNSTYRVVSVLTVL |
| | | HQDWLNGKEYKCKVSNKALPAPIEKTISKAKGQPR |
| | | EPQVYTLPPSREEMTKNQVSLTCLVKGFYPSDIAVE |
| | | WESNGQPENNYKTTPPVLDSDGSFLLYSKLTVDKSR |
| | | WQQGNVFSCSVMHEALHNHYTQKSLSLSPGK |
| EM-1 mAb L1 | SEQ ID NO: 42 | AIQLTQSPSSLSASVGDRVTITCRASQDISSALVWYQ |
| | | QKPGKAPKLLIYDASSLESGVPSRFSGSESGTDFTLTI |
| | | SSLQPEDFATYYCQQFNSYPLTFGGGTKVEIKRTVA |
| | | APSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| EM-1 mAB H2 | SEQ ID NO: 43 | QVQLVQSGAEVKKPGASVKVSCETSGYTFTSYGIS |
| (K409R, anti-cMet) | | WVRQAPGHGLEWMGWISAYNGYTNYAQKLQGRV |
| | | TMTTDTSTSTAYMELRSLRSDDTAVYYCARDLRGT |
| | | NYFDYWGQGTLVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPSNT |
| | | KVDKRVEPKSCDKTHTCPPCPAPELLGGPSVFLFPP |
| | | KPKDTLMISRTPEVTCVVVDVSHEDPEVKFNWYVD |
| | | GVEVHNAKTKPREEQYNSTYRVVSVLTVLHQDWL |
| | | NGKEYKCKVSNKALPAPIEKTISKAKGQPREPQVYT |
| | | LPPSREEMTKNQVSLTCLVKGFYPSDIAVEWESNGQ |
| | | PENNYKTTPPVLDSDGSFFLYSRLTVDKSRWQQGN |
| | | VFSCSVMHEALHNHYTQKSLSLSPGK |
| EM-1 mAb L2 | SEQ ID NO: 44 | DIQMTQSPSSVSASVGDRVTITCRASQGISNWLAWF |
| | | QHKPGKAPKLLIYAASSLLSGVPSRFSGSGSGTDFTL |
| | | TISSLQPEDFATYYCQQANSFPITFGQGTRLEIKRTV |
| | | AAPSVFIFPPSDEQLKSGTASVVCLLNNFYPREAKV |
| | | QWKVDNALQSGNSQESVTEQDSKDSTYSLSSTLTLS |
| | | KADYEKHKVYACEVTHQGLSSPVTKSFNRGEC |

### EXAMPLES

These examples are provided for illustrative purposes only and not to limit the scope of the claims provided herein.

### Example 1: Analysis of Circulating Tumor DNA (ctDNA) from the Phase 2 BLC2001 Trial of Erdafitinib in Locally Advanced or Metastatic Urothelial Carcinoma (mUC) to Identify Markers of Intrinsic Resistance to FGFR-Targeted Therapy

To identify markers of intrinsic resistance to FGFR inhibition, plasma samples from a phase 2, multicenter, open-label study (NCT02365597) of erdafitinib in patients with locally advanced or metastatic urothelial cancer (mUC) and FGFR2/3 alterations (mutations/fusions) were assessed for the presence of gene alterations in circulating tumor DNA (ctDNA) by next-generation sequencing.

Methods

Data Curation

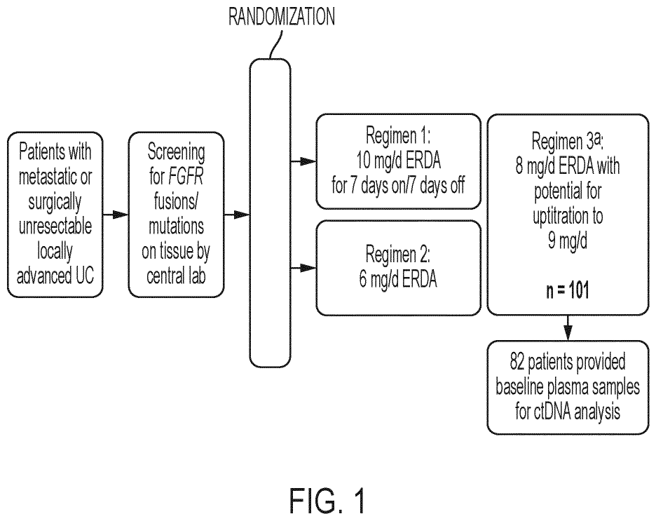

Pre-treatment plasma samples from 155 patients from the Phase 2 study of erdafitinib in advanced UC patients with FGFR mutations or fusions (NCT02365597) were assessed for somatic alterations in ctDNA using next-generation sequencing (NGS). Downstream analysis population centered on 82 patients who received 8 mg of erdafitinib daily, with potential for uptitration to 9 mg daily, reflecting the clinically relevant dosing regimen, with valid clinical responses (FIG. 1).

NGS was performed utilizing the GUARDANT360@ test, which provides complete sequencing of covered exons across 73 genes. Mutation data were filtered to remove germline and synonymous mutations, then selected for likely driver events (i.e. pathogenic variants) using OncoKB (www.oncokb.org) and Cancer Hotspots (www.cancerhotspots.org) knowledge bases. No filtering was applied to fusions or amplifications. Single nucleotide variants (SNV) were included if the affected amino acid position for the gene was reported in either of the knowledge bases. All amplification, splicing, fusion, and promoter mutations were included, independent of annotation in the knowledge bases. All frameshift and nonsense mutations were considered pathogenic unless the gene was preassigned as an oncogene (EGFR, FGFR1, FGFR2, FGFR3, FGFR4, RET, PIK3CA, NRAS, KIT, ErbB2, BRAF, AR, MET, MYC, KIT, PDGFRA).

Statistical Analyses

To be included in downstream association analyses, each gene was required to have at least three patients with pathogenic alterations detected in pre-treatment plasma. The relationship of pre-treatment gene alterations with clinical response to erdafitinib was assessed using Fisher's Exact test. Association of pre-treatment alteration status with patient progression-free survival (PFS) and overall survival (OS) was assessed using Cox Proportional-Hazards models. P-values were adjusted to control the false discovery rate (q) using the Benjamini-Hochberg method.

Results

Association of Altered Genes with Clinical Response

The association between alterations in any of the 72 genes screened and best overall response (BOR) to erdafitinib was assessed, comparing observed alterations in the responsive (complete responder (CR) and partial responder (PR)) group with patients with BOR of progressive disease (PD). No genes were significantly associated with BOR of PD in response to erdafitinib. ARID1A alterations showed significant association with BOR of PD as assessed by nominal, but not adjusted, p value (Table 7).

TABLE 7
Baseline alterations and BOR: CR/PR vs. PD

| | | P | Adjusted | Lower | Upper |
|---|---|---|---|---|---|
| Gene^{a} | Estimate | value | P value | CI | CI |
| ARID1A (N = 8) | 5.121 | 0.04259 | 0.4505 | 0.8305 | 39.04 |
| TP53 (N = 29) | 4.118 | 0.0606 | 0.4505 | 0.8931 | 26.7 |
| CCND1 (N = 4) | 8.081 | 0.07508 | 0.4505 | 0.5862 | 457.5 |
| EGFR (N = 5) | 3.99 | 0.1517 | 0.6321 | 0.4056 | 53.26 |
| ERBB2 (N = 3) | 5.033 | 0.2107 | 0.6321 | 0.2429 | 316.7 |

^{a}Does not include patients with BOR of stable disease; Fisher's exact test.

Association of Altered Genes with PFS and OS

The presence of genetic alterations at baseline and association with PFS and OS outcomes in erdafitinib-treated patients was assessed.

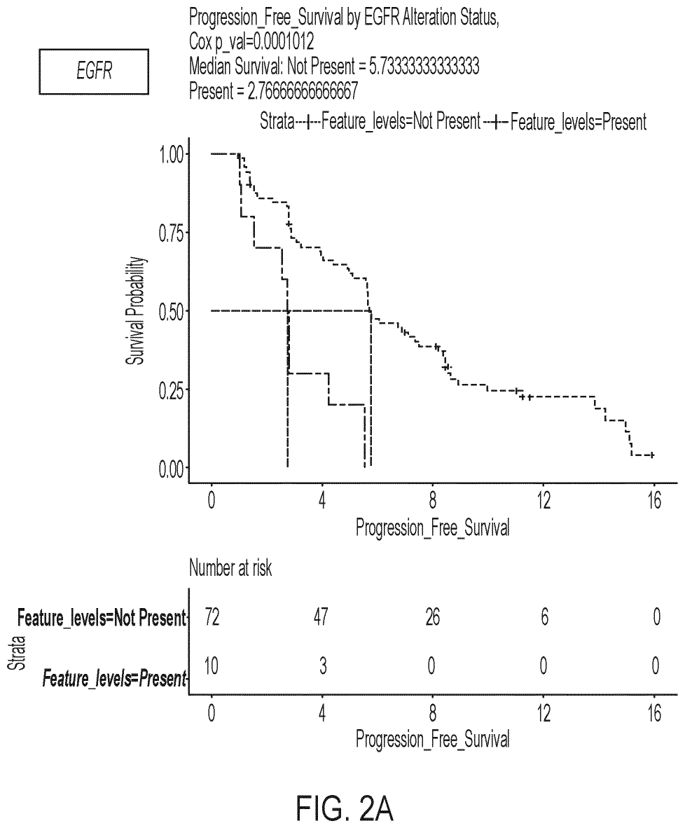

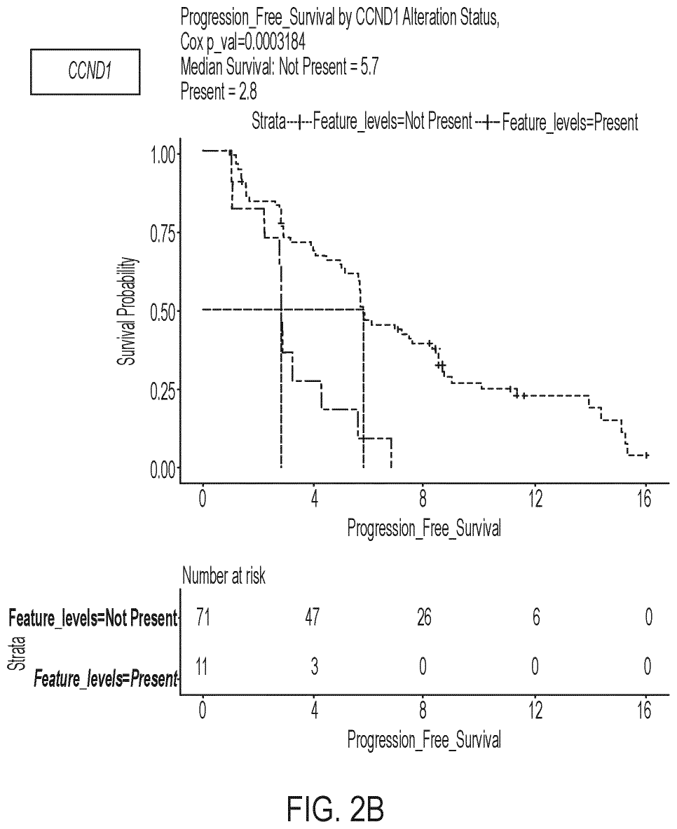

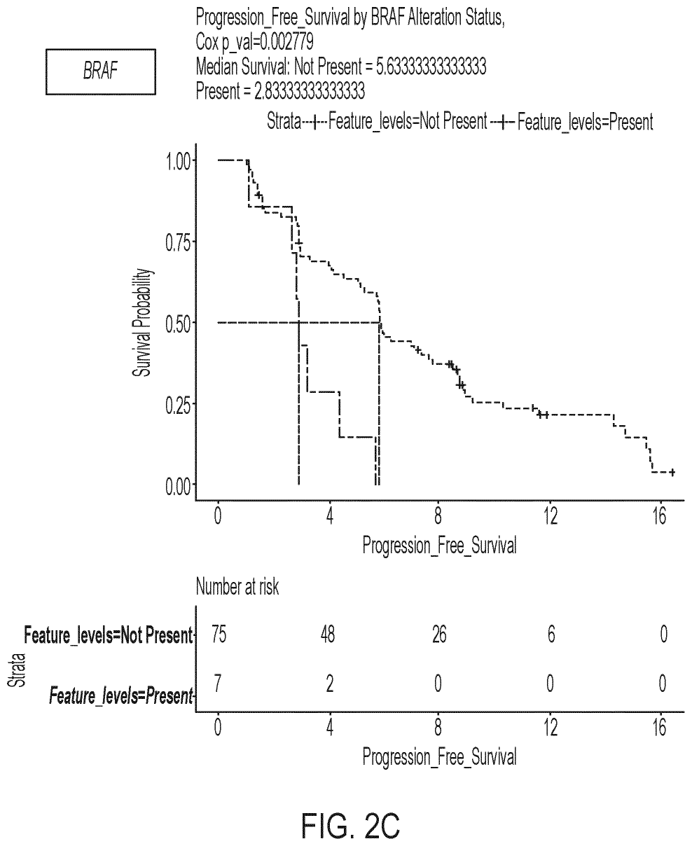

Patients with alterations in EGFR, CCND1 or BRAF in pre-treatment plasma exhibited significantly shorter PFS compared with patients negative for alterations in these genes. Patients with EGFR alterations at baseline had a median PFS of 2.8 months compared with 5.7 months in alteration-negative patients (HR, 4.3; 95% CI, 2.1-8.9; q=0.0026) (Table 8 and FIG. 2A). Similarly, patients with CCND1 (2.8 vs 5.7 months; HR, 3.6; 95% CI, 1.8-7.1; q=0.0041) (Table 8 and FIG. 2B), and BRAF (2.8 vs 5.6 months; HIR, 3.6; 95% CI, 1.6-8.2; q=0.024) (Table 8 and FIG. 2C) alterations at baseline exhibited shorter median PFS compared with alteration-negative patients.

TABLE 8
Altered Gene Association with PFS

| | Median PFS (months) | | | | |
|---|---|---|---|---|---|
| | Alteration | Alteration | Hazard Ratio | Nominal | Adjusted |
| Gene^{a} | (−) | (+) | (95% CI) | p-value | p-value |
| EGFR (N = 10) | 5.73 | 2.77 | 4.27 (2.05-8.89) | 0.00010 | 0.0026 |
| CCND1 (N = 11) | 5.70 | 2.80 | 3.56 (1.78-7.09) | 0.00032 | 0.0041 |
| BRAF (N = 7) | 5.63 | 2.83 | 3.56 (1.55-8.17) | 0.0028 | 0.024 |
| FGFR1-Amp | 5.63 | 2.80 | 3.57 (1.37-9.33) | 0.0094 | 0.061 |
| ARID1A (N = 12) | 5.63 | 2.77 | 2.25 (1.13-4.5) | 0.021 | 0.092 |
| ERBB2 (N = 3) | 5.63 | 1.37 | 4.03 (1.23-13.2) | 0.021 | 0.092 |
| TP53 | 6.73 | 4.23 | 1.69 (1.03-2.76) | 0.037 | 0.14 |

^{a}Genes with nominal p value < 0.05

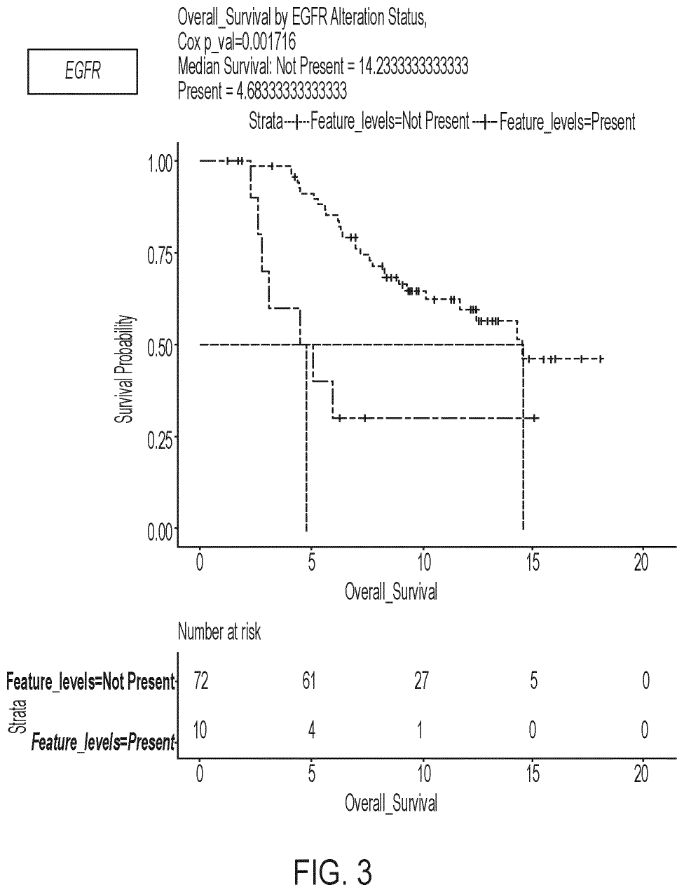

Patients with EGFR alterations also had significantly shorter median OS compared with patients without EGFR alterations (4.7 months vs 14.2 months; HIR, 3.9; 95% CI, 1.7-9.0; q=0.045) (Table 9 and FIG. 3).

TABLE 9
Altered Gene Association with OS

| | Median OS (months) | | | | |
|---|---|---|---|---|---|
| | Alteration | Alteration | Hazard Ratio | Nominal | Adjusted |
| Gene^{a} | (−) | (+) | (95% CI) | p-value | p-value |
| EGFR (N = 10) | 14.23 | 4.68 | 3.88 (1.66-9.04) | 0.0017 | 0.045 |
| CCND1 (N = 11) | 14.23 | 5.57 | 2.89 (1.25-6.67) | 0.013 | 0.17 |
| TERT | N/A | 9.10 | 2.04 (1.04-3.98) | 0.038 | 0.19 |
| BRAF (N = 7) | 14 | 4.97 | 2.92 (1.01-8.44) | 0.048 | 0.19 |

^{a}Genes with nominal p value < 0.05

Frequency of Markers of Intrinsic Resistance

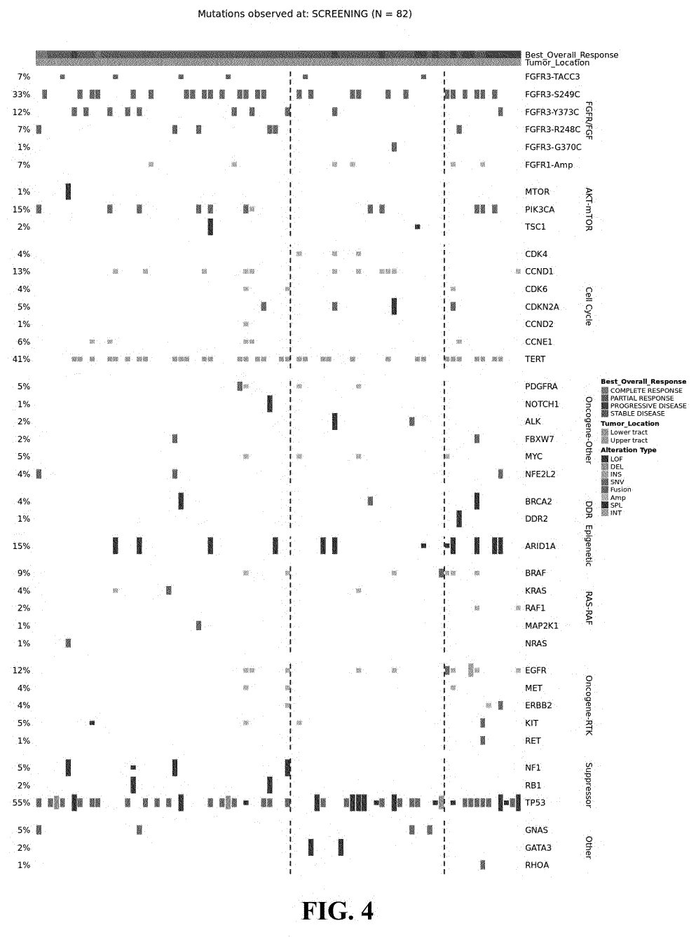

Among 82 patients in the 8 mg/day erdafitinib cohort with potential for uptitration, EGFR (n=10, 120%), CCND1 (n=11, 130%), or BRAF (n=7, 90%) alterations were observed in 21% (17/82) of patients (Table 10, FIG. 4). Amplifications were the predominant alteration with 8/10 (800%), 11/11 (1000%), and 6/7 (860%) patients exhibiting amplification in EGFR, CCND1, or BRAF genes, respectively (Table 10).

TABLE 10
Gene Amplification was Predominant Among
Genes Associated with Shorter PFS and OS

| | Altered, | Amplified^{a}, | Amplified, | |
|---|---|---|---|---|
| | n (% of 82 | n/n (% of | n (% of 82 | FI CNV |
| | patients) | alterations) | patients) | frequency^{b} (%) |
| EGFR | 10 (12) | 8/10 (80) | 8 (10) | 3 |
| CCND1 | 11 (13) | 11/11 (100) | 11 (13) | 13 |
| BRAF | 7 (9) | 6/7 (86) | 6 (7) | 0 |

^{a}Observed non-amplification alterations. EGFR (n = 2): N771_H773dup insertion; and K80N mutation. BRAF (n = 1): concurrent mutations D594G and K601E.
^{b}FI-derived gene alterations via tissue-based testing whereas gene alterations in the study were determined by blood-based testing.

Co-expression of altered resistance genes was observed (Table 11).

TABLE 11
Subject Count for Mutation Co-occurrence

| # Genes* with Mutation | 0 | 1 | 2 | 3 |
|---|---|---|---|---|
| # of Subjects | 65 | 8 | 7 | 2 |
| % of Subjects | 79.3% | 9.8% | 8.5% | 2.4% |

*EGFR, CCND1, BRAF

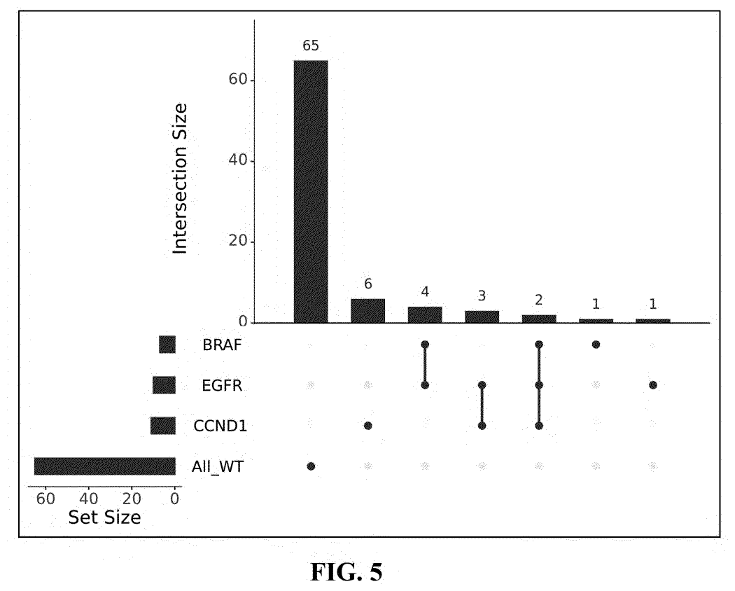

Concomitant alterations in EGFR and BRAF were observed at baseline in 4 patients; EGFR and CCND1 in 3 patients; and in EGFR, BRAF and CCND1 in 2 patients (FIG. 5).

### SUMMARY AND CONCLUSIONS

In patients with locally advanced or mUC and FGFR2/3 alterations, the presence of EGFR, CCND1, and BRAF alterations at baseline correlated with shorter PFS; and EGFR alterations at baseline correlated with shorter OS. Gene amplifications were the predominant alterations observed in this FGFR alteration-positive population.

Patients with alterations in EGFR, CCND1 and BRAF did respond to erdafitinib. These results indicate that combination therapy may further benefit patients with alterations in both FGFR and one of these genes. Specifically, the combination of erdafitinib with EGFR, CCND1 or BRAF inhibitors or inhibitors of these pathways. Combination therapy could benefit the 21% of patients on study whom had alteration in at least one of these genes.

The examples and embodiments described herein are for illustrative purposes only and various modifications or changes suggested to persons skilled in the art are to be included within the spirit and purview of this application and scope of the appended claims.

What is claimed:

1. A method of treating cancer comprising administering a fibroblast growth factor receptor (FGFR) inhibitor in combination with an epidermal growth factor receptor (EGFR) inhibitor to a patient in need of cancer treatment, wherein the patient harbors at least one FGFR2 genetic alteration or FGFR3 genetic alteration and at least one EGFR genetic alteration.

2. The method of claim 1, wherein the cancer is urothelial carcinoma.

3. The method of claim 2, wherein the urothelial carcinoma is locally advanced or metastatic.

4. The method of any one of the preceding claims, wherein administration of the FGFR inhibitor in combination with an EGFR inhibitor provides improved anti-tumor activity, as measured by overall survival or progression-free survival, relative to a patient with cancer who was not receiving treatment with an FGFR inhibitor in combination with an EGFR inhibitor.

5. The method of any one of the preceding claims, wherein the patient is resistant to treatment with erdafitinib or has acquired resistance to treatment with erdafitinib.

6. The method of any one of the preceding claims, wherein the patient received at least one systemic therapy for the treatment of urothelial carcinoma prior to administration of said FGFR inhibitor and said EGFR inhibitor.

7. The method of claim 6, wherein the at least one systemic therapy for the treatment of urothelial carcinoma is platinum-containing chemotherapy.

8. The method of claim 7, wherein the urothelial carcinoma progressed during or following at least one line of the platinum-containing chemotherapy.

9. The method of any one of the preceding claims, wherein the FGFR2 genetic alteration is a gene fusion.

10. The method of claim 9, wherein the FGFR2 gene fusion is FGFR2-BICC1, FGFR2-CASP7, or any combination thereof.

11. The method of any one of the preceding claims, wherein the FGFR3 genetic alteration is a gene fusion.

12. The method of claim 11, wherein FGFR3 gene fusion is FGFR3-TACC3, FGFR3-BAIAP2L1, or any combination thereof.

13. The method of any one of the preceding claims, wherein the FGFR3 genetic alteration is a gene mutation.

14. The method of claim 13, wherein the FGFR3 gene mutation is R248C, S249C, G370C, Y373C, or any combination thereof.

15. The method of any one of the preceding claims, wherein the EGFR genetic alteration is a gene amplification, a gene mutation, a gene insertion, or any combination thereof.

16. The method of claim 15, wherein the EGFR gene mutation is a K80N substitution.

17. The method of claim 15, wherein the EGFR gene insertion is an N771_H773dup insertion.

18. The method of any one of the preceding claims, wherein the patient also harbors at least one CCND1 genetic alteration.

19. The method of claim 18, wherein the CCND1 genetic alteration is a gene amplification.

20. The method of claim 18 or 19, further comprising administering to said patient a CCND1 inhibitor.

21. The method of any one of the preceding claims, wherein the patient also harbors at least one BRAF genetic alteration.

22. The method of claim 21, wherein the BRAF genetic alteration is a gene amplification, a gene mutation, a gene insertion, or any combination thereof.

23. The method of claim 22, wherein the BRAF gene mutation is a D594G substitution, a K601E substitution, or any combination thereof.

24. The method of any one of claims 21 to 23, further comprising administering to said patient a BRAF inhibitor.

25. The method of any one of the preceding claims, further comprising evaluating a biological sample from the patient for the presence of at least one FGFR2 genetic alteration or FGFR3 genetic alteration and at least one EGFR genetic alteration prior to administration of said FGFR inhibitor and said EGFR inhibitor.

26. The method of claim 25, wherein the biological sample is blood, lymph fluid, bone marrow, a solid tumor sample, or any combination thereof.

27. The method of any one of the preceding claims, wherein the FGFR inhibitor is erdafitinib.

28. The method of claim 27, wherein erdafitinib is administered daily.

29. The method of claim 27 or 28, wherein erdafitinib is administered orally.

30. The method of any one of claims 27 to 29, wherein erdafitinib is administered orally on a continuous daily dosing schedule.

31. The method of any one of claims 27 to 30, wherein erdafitinib is administered orally at a dose of about 8 mg once daily.

32. The method of claim 31, wherein the dose of erdafitinib is increased from 8 mg once daily to 9 mg once daily if:
(a) the patient exhibits a serum phosphate (PO₄) level that is less than about 5.5 mg/dL at 14-21 days after initiating treatment; and
(b) administration of erdafitinib at 8 mg once daily resulted in no ocular disorder; or
(c) administration of erdafitinib at 8 mg once daily resulted in no Grade 2 or greater adverse reaction,
wherein the increase from 8 mg once daily to 9 mg once daily begins at 14 to 21 days after initiating treatment.

33. The method of any one of claims 27 to 32, wherein erdafitinib is present in a solid dosage form.

34. The method of claim 33, wherein the solid dosage form is a tablet.

35. The method of any one of the preceding claims, wherein the EGFR inhibitor is an isolated bispecific epidermal growth factor receptor (EGFR)/hepatocyte growth factor receptor (c-Met) antibody.

36. The method of claim 35, wherein the EGFR/c-Met antibody comprises a first heavy chain (HC1), a first light chain (LC1), a second heavy chain (HC2) and a second light chain (LC2), wherein the HC1, the LC1, the HC2 and the LC2 comprise the amino acid sequences of SEQ ID NOs: 41, 42, 43, and 44, respectively.

37. The method of any one of the preceding claims, wherein the FGFR inhibitor and the EGFR inhibitor are administered simultaneously, concurrently, or sequentially.

38. A method of treating cancer in a patient comprising:
(a) evaluating a biological sample from the patient for the presence of at least one fibroblast growth factor receptor 2 (FGFR2) genetic alteration or fibroblast growth factor receptor 3 (FGFR3) genetic alteration and at least one EGFR genetic alteration; and
(b) treating the patient with an FGFR inhibitor in combination with an EGFR inhibitor if the at least one FGFR2 genetic alteration or FGFR3 genetic alteration and the at least one EGFR genetic alteration are present in the sample.

39. A method of predicting duration of progression-free survival in a human patient having cancer, the method comprising evaluating a biological sample from the patient for the presence of at least one FGFR2 genetic alteration or FGFR3 genetic alteration and at least one EGFR genetic alteration, wherein the presence of at least one FGFR2 genetic alteration or FGFR3 genetic alteration and at least one EGFR genetic alteration indicates a shorter duration of progression free survival, relative to a human patient having cancer who does not harbor at least one EGFR genetic alteration.

40. The method of claim 39, further comprising administering to the patient an FGFR inhibitor in combination with an EGFR inhibitor if the at least one FGFR2 genetic alteration or FGFR3 genetic alteration and the at least one EGFR genetic alteration are present in the sample.

41. The method of claim 39 or 40, wherein the patient is resistant to treatment with erdafitinib or has acquired resistance to treatment with erdafitinib.

42. The method of any one of claims 39 to 41, wherein the patient also harbors a BRAF genetic alteration, a Cyclin D1 (CCND1) genetic alteration, or any combination thereof.

43. A method of predicting duration of overall survival in a human patient having cancer, the method comprising evaluating a biological sample from the patient for the presence of at least one FGFR2 genetic alteration or FGFR3 genetic alteration and at least one EGFR genetic alteration, wherein the presence of at least one FGFR2 genetic alteration or FGFR3 genetic alteration and at least one EGFR genetic alteration indicates a shorter duration of overall survival relative to a human patient having cancer who does not harbor at least one EGFR genetic alteration.

44. The method of claim 43, further comprising administering to the patient an FGFR inhibitor in combination with an EGFR inhibitor if the at least one FGFR2 genetic alteration or FGFR3 genetic alteration and the at least one EGFR genetic alteration are present in the sample.

45. The method of claim 43 or 44, wherein the patient is resistant to treatment with erdafitinib or has acquired resistance to treatment with erdafitinib.

46. A method of improving overall survival in a patient with cancer relative to a patient with cancer who was not receiving treatment with a fibroblast growth factor receptor (FGFR) inhibitor in combination with an epidermal growth factor receptor (EGFR) inhibitor, said method comprising providing to said patient an FGFR inhibitor in combination with an EGFR inhibitor, wherein said patient harbors at least one FGFR2 genetic alteration or FGFR3 genetic alteration and at least one EGFR genetic alteration.

47. A method of improving progression-free survival in a patient with cancer relative to a patient with urothelial carcinoma who was not receiving treatment with a fibroblast growth factor receptor (FGFR) inhibitor in combination with an epidermal growth factor receptor (EGFR) inhibitor, said method comprising providing to said patient an FGFR inhibitor in combination with an EGFR inhibitor, wherein said patient harbors at least one FGFR2 genetic alteration or FGFR3 genetic alteration and at least one EGFR genetic alteration.

48. A fibroblast growth factor receptor (FGFR) inhibitor and epidermal growth factor receptor (EGFR) inhibitor for use in the treatment of cancer in a patient who harbors at least one FGFR2 genetic alteration or FGFR3 genetic alteration and at least one EGFR genetic alteration.

49. The use of a fibroblast growth factor receptor (FGFR) inhibitor for the manufacture of a medicament for the treatment of cancer in a patient who harbors at least one FGFR2 genetic alteration or FGFR3 genetic alteration and at least one epidermal growth factor receptor (EGFR) genetic alteration, wherein the FGFR inhibitor is to be used in combination with an EGFR inhibitor.

50. The use of epidermal growth factor receptor (EGFR) inhibitor for the manufacture of a medicament for the treatment of urothelial carcinoma in a patient who harbors at least one fibroblast growth factor receptor 2 (FGFR2) genetic alteration or fibroblast growth factor receptor 3 (FGFR3) genetic alteration and at least one EGFR genetic alteration, wherein the EGFR inhibitor is to be used in combination with an FGFR inhibitor.

51. A method of treating cancer comprising administering a fibroblast growth factor receptor (FGFR) inhibitor in combination with a Cyclin D1 (CCND1) inhibitor to a patient in need of cancer treatment, wherein the patient harbors at least one FGFR2 genetic alteration or FGFR3 genetic alteration and at least one CCND1 genetic alteration.

52. The method of claim 51, wherein the cancer is urothelial carcinoma.

53. The method of claim 52, wherein the urothelial carcinoma is locally advanced or metastatic.

54. The method of any one of claims 51 to 53, wherein administration of the FGFR inhibitor in combination with a CCND1 inhibitor provides improved anti-tumor activity as measured by progression-free survival relative to a patient with cancer who was not receiving treatment with an FGFR inhibitor in combination with a CCND1 inhibitor.

55. The method of any one of claims 51 to 54, wherein the patient is resistant to treatment with erdafitinib or has acquired resistance to treatment with erdafitinib.

56. The method of any one of claims 51 to 55, wherein the patient received at least one systemic therapy for the treatment of urothelial carcinoma prior to administration of said FGFR inhibitor and said CCND1 inhibitor.

57. The method of claim 56, wherein the at least one systemic therapy for the treatment of urothelial carcinoma is platinum-containing chemotherapy.

58. The method of claim 57, wherein the urothelial carcinoma progressed during or following at least one line of the platinum-containing chemotherapy.

59. The method of any one of claims 51 to 58, wherein the FGFR2 genetic alteration is a gene fusion.

60. The method of claim 59, wherein the FGFR2 gene fusion is FGFR2-BICC1, FGFR2-CASP7, or any combination thereof.

61. The method of any one of claims 51 to 60, wherein the FGFR3 genetic alteration is a gene fusion.

62. The method of claim 61, wherein FGFR3 gene fusion is FGFR3-TACC3, FGFR3-BAIAP2L1, or any combination thereof.

63. The method of any one of claims 51 to 62, wherein the FGFR3 genetic alteration is a gene mutation.

64. The method of claim 63, wherein the FGFR3 gene mutation is R248C, S249C, G370C, Y373C, or any combination thereof.

65. The method of any one of claims 51 to 64, wherein the CCND1 genetic alteration is a gene amplification.

66. The method of any one of claims 51 to 65, wherein the patient also harbors at least one EGFR genetic alteration.

67. The method of claim 66, wherein the EGFR genetic alteration is a gene amplification, a gene mutation, a gene insertion, or any combination thereof.

68. The method of claim 67, wherein the EGFR gene mutation is a K80N substitution.

69. The method of claim 67, wherein the EGFR gene insertion is an N771_H773dup insertion.

70. The method of any one of claims 66 to 69, further comprising administering to said patient an EGFR inhibitor.

71. The method of any one of claims 51 to 70, wherein the patient also harbors at least one BRAF genetic alteration.

72. The method of claim 71, wherein the BRAF genetic alteration is a gene amplification, a gene mutation, a gene insertion, or any combination thereof.

73. The method of claim 72, wherein the BRAF gene mutation is a D594G substitution, a K601E substitution, or any combination thereof.

74. The method of any one of claims 71 to 73, further comprising administering to said patient a BRAF inhibitor.

75. The method of any one of claims 51 to 74, further comprising evaluating a biological sample from the patient for the presence of at least one FGFR2 genetic alteration or FGFR3 genetic alteration and at least one CCND1 genetic alteration prior to administration of said FGFR inhibitor and said CCND1 inhibitor.

76. The method of claim 75, wherein the biological sample is blood, lymph fluid, bone marrow, a solid tumor sample, or any combination thereof.

77. The method of any one of claims 51 to 76, wherein the FGFR inhibitor is erdafitinib.

78. The method of claim 77, wherein erdafitinib is administered daily.

79. The method of claim 77 or 78, wherein erdafitinib is administered orally.

80. The method of any one of claims 77 to 79, wherein erdafitinib is administered orally on a continuous daily dosing schedule.

81. The method of any one of claims 77 to 80, wherein erdafitinib is administered orally at a dose of about 8 mg once daily.

82. The method of claim 81, wherein the dose of erdafitinib is increased from 8 mg once daily to 9 mg once daily if:
(a) the patient exhibits a serum phosphate (PO₄) level that is less than about 5.5 mg/dL at 14-21 days after initiating treatment; and
(b) administration of erdafitinib at 8 mg once daily resulted in no ocular disorder; or
(c) administration of erdafitinib at 8 mg once daily resulted in no Grade 2 or greater adverse reaction,
wherein the increase from 8 mg once daily to 9 mg once daily begins at 14 to 21 days after initiating treatment

83. The method of any one of claims 77 to 82, wherein erdafitinib is present in a solid dosage form.

84. The method of claim 83, wherein the solid dosage form is a tablet.

85. The method of any one of claims 51 to 84, wherein the FGFR inhibitor and the CCND1 inhibitor are administered simultaneously, concurrently, or sequentially.

86. A method of treating cancer in a patient comprising:
(a) evaluating a biological sample from the patient for the presence of at least one fibroblast growth factor receptor 2 (FGFR2) genetic alteration or fibroblast growth factor receptor 3 (FGFR3) genetic alteration and at least one Cyclin D1 (CCND1) genetic alteration; and
(b) treating the patient with an FGFR inhibitor in combination with a CCND1 inhibitor if the at least one FGFR2 genetic alteration or FGFR3 genetic alteration and the at least one CCND1 genetic alteration are present in the sample.

87. A method of predicting duration of progression-free survival in a human patient having cancer, the method comprising evaluating a biological sample from the patient for the presence of at least one fibroblast growth factor receptor 2 (FGFR2) genetic alteration or fibroblast growth factor receptor 3 (FGFR3) genetic alteration and at least one Cyclin D1 (CCND1) genetic alteration, wherein the presence of at least one FGFR2 genetic alteration or FGFR3 genetic alteration and at least one CCND1 genetic alteration indicates a shorter duration of progression free survival relative to a human patient having cancer who does not harbor at least one CCND1 genetic alteration.

88. The method of claim 87, further comprising administering to the patient an FGFR inhibitor in combination with a CCND1 inhibitor if the at least one FGFR2 genetic alteration or FGFR3 genetic alteration and the at least one CCND1 genetic alteration are present in the sample.

89. The method of claim 87 or 88, wherein the patient is resistant to treatment with erdafitinib or has acquired resistance to treatment with erdafitinib.

90. The method of any one of claims 87 to 89, wherein the patient also harbors a BRAF genetic alteration, an EGFR genetic alteration, or any combination thereof.

91. A method of improving progression-free survival in a patient with cancer relative to a patient with cancer who was not receiving treatment with a fibroblast growth factor receptor (FGFR) inhibitor in combination with a Cyclin D1 (CCND1) inhibitor, said method comprising providing to said patient an FGFR inhibitor in combination with a CCND1 inhibitor, wherein said patient harbors at least one FGFR2 genetic alteration or FGFR3 genetic alteration and at least one CCND1 genetic alteration.

92. A fibroblast growth factor receptor (FGFR) inhibitor and Cyclin D1 (CCND1) inhibitor for use in the treatment of cancer in a patient who harbors at least one FGFR2 genetic alteration or FGFR3 genetic alteration and at least one CCND1 genetic alteration.

93. The use of a fibroblast growth factor receptor (FGFR) inhibitor for the manufacture of a medicament for the treatment of cancer in a patient who harbors at least one FGFR2 genetic alteration or FGFR3 genetic alteration and at least one Cyclin D1 (CCND1) genetic alteration, wherein the FGFR inhibitor is to be used in combination with a CCND1 inhibitor.

94. The use of Cyclin D1 (CCND1) inhibitor for the manufacture of a medicament for the treatment of urothelial carcinoma in a patient who harbors at least one fibroblast growth factor receptor 2 (FGFR2) genetic alteration or fibroblast growth factor receptor 3 (FGFR3) genetic alteration and at least one CCND1 genetic alteration, wherein the CCND1 inhibitor is to be used in combination with an FGFR inhibitor.

95. A method of treating cancer comprising administering a fibroblast growth factor receptor (FGFR) inhibitor in combination with a BRAF inhibitor to a patient in need of cancer treatment, wherein the patient harbors at least one FGFR2 genetic alteration or FGFR3 genetic alteration and at least one BRAF genetic alteration.

96. The method of claim 95, wherein the cancer is urothelial carcinoma.

97. The method of claim 96, wherein the urothelial carcinoma is locally advanced or metastatic.

98. The method of any one of claims 95 to 97, wherein administration of the FGFR inhibitor in combination with a BRAF inhibitor provides improved anti-tumor activity as measured by progression-free survival relative to a patient with cancer who was not receiving treatment with an FGFR inhibitor in combination with a BRAF inhibitor.

99. The method of any one of claims 95 to 98, wherein the patient is resistant to treatment with erdafitinib or has acquired resistance to treatment with erdafitinib.

100. The method of any one of claims 95 to 99, wherein the patient received at least one systemic therapy for the treatment of urothelial carcinoma prior to administration of said FGFR inhibitor and said BRAF inhibitor.

101. The method of claim 100, wherein the at least one systemic therapy for the treatment of urothelial carcinoma is platinum-containing chemotherapy.

102. The method of claim 101, wherein the urothelial carcinoma progressed during or following at least one line of the platinum-containing chemotherapy.

103. The method of any one of claims 95 to 102, wherein the FGFR2 genetic alteration is a gene fusion.

104. The method of claim 103, wherein the FGFR2 gene fusion is FGFR2-BICC1, FGFR2-CASP7, or any combination thereof.

105. The method of any one of claims 95 to 104, wherein the FGFR3 genetic alteration is a gene fusion.

106. The method of claim 105, wherein FGFR3 gene fusion is FGFR3-TACC3, FGFR3-BAIAP2L1, or any combination thereof.

107. The method of any one of claims 95 to 106, wherein the FGFR3 genetic alteration is a gene mutation.

108. The method of claim 107, wherein the FGFR3 gene mutation is R248C, S249C, G370C, Y373C, or any combination thereof.

109. The method of any one of claims 95 to 108, wherein the BRAF genetic alteration is a gene amplification, a gene mutation, a gene insertion, or any combination thereof.

110. The method of claim 109, wherein the BRAF gene mutation is a D594G substitution, a K601E substitution, or any combination thereof

111. The method of any one of claims 95 to 110, wherein the patient also harbors at least one CCND1 genetic alteration.

112. The method of claim 111, wherein the CCND1 genetic alteration is a gene amplification.

113. The method of claim 11 or 112, further comprising administering to said patient a CCND1 inhibitor.

114. The method of any one of claims 95 to 113, wherein the patient also harbors at least one EGFR genetic alteration.

115. The method of claim 114, wherein the EGFR genetic alteration is a gene amplification, a gene mutation, a gene insertion, or any combination thereof.

116. The method of claim 115, wherein the EGFR gene mutation is a K80N substitution.

117. The method of claim 115, wherein the EGFR gene insertion is an N771_H773dup insertion.

118. The method of any one of claims 114 to 117, further comprising administering to said patient an EGFR inhibitor.

119. The method of any one of claims 95 to 118, further comprising evaluating a biological sample from the patient for the presence of at least one FGFR2 genetic alteration or FGFR3 genetic alteration and at least one BRAF genetic alteration prior to administration of said FGFR inhibitor and said BRAF inhibitor.

120. The method of claim 119, wherein the biological sample is blood, lymph fluid, bone marrow, a solid tumor sample, or any combination thereof.

121. The method of any one of claims 95 to 120, wherein the FGFR inhibitor is erdafitinib.

122. The method of claim 121, wherein erdafitinib is administered daily.

123. The method of claim 121 or 122, wherein erdafitinib is administered orally.

124. The method of any one of claims 121 to 123, wherein erdafitinib is administered orally on a continuous daily dosing schedule.

125. The method of any one of claims 121 to 124, wherein erdafitinib is administered orally at a dose of about 8 mg once daily.

126. The method of claim 125, wherein the dose of erdafitinib is increased from 8 mg once daily to 9 mg once daily if:
(a) the patient exhibits a serum phosphate (PO₄) level that is less than about 5.5 mg/dL at 14-21 days after initiating treatment; and
(b) administration of erdafitinib at 8 mg once daily resulted in no ocular disorder; or
(c) administration of erdafitinib at 8 mg once daily resulted in no Grade 2 or greater adverse reaction,
wherein the increase from 8 mg once daily to 9 mg once daily begins at 14 to 21 days after initiating treatment.

127. The method of any one of claims 121 to 126, wherein erdafitinib is present in a solid dosage form.

128. The method of claim 127, wherein the solid dosage form is a tablet.

129. The method of any one of claims 121 to 128, wherein the FGFR inhibitor and the BRAF inhibitor are administered simultaneously, concurrently, or sequentially.

130. A method of treating cancer in a patient comprising:
(a) evaluating a biological sample from the patient for the presence of at least one fibroblast growth factor receptor 2 (FGFR2) genetic alteration or fibroblast growth factor receptor 3 (FGFR3) genetic alteration and at least one BRAF genetic alteration; and
(b) treating the patient with an FGFR inhibitor in combination with a BRAF inhibitor if the at least one FGFR2 genetic alteration or FGFR3 genetic alteration and the at least one BRAF genetic alteration are present in the sample.

131. A method of predicting duration of progression-free survival in a human patient having cancer, the method comprising evaluating a biological sample from the patient for the presence of at least one fibroblast growth factor receptor 2 (FGFR2) genetic alteration or fibroblast growth factor receptor 3 (FGFR3) genetic alteration and at least one BRAF genetic alteration, wherein the presence of at least one FGFR2 genetic alteration or FGFR3 genetic alteration and at least one BRAF genetic alteration indicates a shorter duration of progression free survival relative to a human patient having cancer who does not harbor at least one BRAF genetic alteration.

132. The method of claim 131, further comprising administering to the patient an FGFR inhibitor in combination with a BRAF inhibitor if the at least one FGFR2 genetic alteration or FGFR3 genetic alteration and the at least one BRAF genetic alteration are present in the sample.

133. The method of claim 131 or 132, wherein the patient is resistant to treatment with erdafitinib or has acquired resistance to treatment with erdafitinib.

134. The method of any one of claims 131 to 133, wherein the patient also harbors a CCND1 genetic alteration, an EGFR genetic alteration, or any combination thereof.

135. A method of improving progression-free survival in a patient with cancer relative to a patient with cancer who was not receiving treatment with a fibroblast growth factor receptor (FGFR) inhibitor in combination with a BRAF inhibitor, said method comprising providing to said patient an FGFR inhibitor in combination with a BRAF inhibitor, wherein said patient harbors at least one FGFR2 genetic alteration or FGFR3 genetic alteration and at least one BRAF genetic alteration.

136. A fibroblast growth factor receptor (FGFR) inhibitor and BRAF inhibitor for use in the treatment of cancer in a patient who harbors at least one FGFR2 genetic alteration or FGFR3 genetic alteration and at least one BRAF genetic alteration.

137. The use of a fibroblast growth factor receptor (FGFR) inhibitor for the manufacture of a medicament for the treatment of cancer in a patient who harbors at least one FGFR2 genetic alteration or FGFR3 genetic alteration and at least one BRAF genetic alteration, wherein the FGFR inhibitor is to be used in combination with a BRAF inhibitor.

138. The use of BRAF inhibitor for the manufacture of a medicament for the treatment of urothelial carcinoma in a patient who harbors at least one fibroblast growth factor receptor 2 (FGFR2) genetic alteration or fibroblast growth factor receptor 3 (FGFR3) genetic alteration and at least one BRAF genetic alteration, wherein the BRAF inhibitor is to be used in combination with an FGFR inhibitor.

139. A fibroblast growth factor receptor (FGFR) inhibitor for use in the treatment of cancer in a patient who harbors at least one FGFR2 genetic alteration or FGFR3 genetic alteration and at least one EGFR genetic alteration, wherein the FGFR inhibitor is to be used in combination with an epidermal growth factor receptor (EGFR) inhibitor.

140. An epidermal growth factor receptor (EGFR) inhibitor for use in the treatment of cancer in a patient who harbors at least one FGFR2 genetic alteration or FGFR3 genetic alteration and at least one EGFR genetic alteration, wherein the EGFR inhibitor is to be used in combination with a fibroblast growth factor receptor (FGFR) inhibitor.

141. An inhibitor for use according to claim 139 or 140, wherein the EGFR genetic alteration is a K80N substitution or an N771_H773dup insertion.

142. A fibroblast growth factor receptor (FGFR) inhibitor for use in the treatment of cancer in a patient who harbors at least one FGFR2 genetic alteration or FGFR3 genetic alteration and at least one CCND1 genetic alteration, wherein the FGFR inhibitor is to be used in combination with a Cyclin D1 (CCND1) inhibitor.

143. A Cyclin D1 (CCND1) inhibitor for use in the treatment of cancer in a patient who harbors at least one FGFR2 genetic alteration or FGFR3 genetic alteration and at least one CCND1 genetic alteration, wherein the CCND1 inhibitor is to be used in combination with a fibroblast growth factor receptor (FGFR) inhibitor.

144. An inhibitor for use according to claim 142 or 143, wherein the CCND1 genetic alteration is a gene amplification.

145. A fibroblast growth factor receptor (FGFR) inhibitor for use in the treatment of cancer in a patient who harbors at least one FGFR2 genetic alteration or FGFR3 genetic alteration and at least one BRAF genetic alteration, wherein the FGFR inhibitor is to be used in combination with a BRAF inhibitor.

146. A BRAF inhibitor for use in the treatment of cancer in a patient who harbors at least one FGFR2 genetic alteration or FGFR3 genetic alteration and at least one BRAF genetic alteration, wherein the BRAF inhibitor is to be used in combination with a fibroblast growth factor receptor (FGFR) inhibitor.

147. An inhibitor for use according to claim 145 or 146, wherein the BRAF genetic alteration is a D594G substitution, or a K601E substitution.

148. An inhibitor for use according to any one of claims 48, 92, 136, or 139 to 147, or the use according to any one of claims 49, 50, 93, 94, 137 or 138, wherein the cancer is urothelial carcinoma.

149. An inhibitor for use or use according to claim 148 wherein the urothelial carcinoma is locally advanced or metastatic.

150. The inhibitor for use according to any one of claims 48, 92, 136, or 139 to 149, or the use according to any one of claims 49, 50, 93, 94, 137, 138, 148 or 149 wherein the FGFR2 genetic alteration is a gene fusion.

151. The inhibitor for use or the use according to claim 150, wherein the FGFR2 gene fusion is FGFR2-BICC1, FGFR2-CASP7, or any combination thereof.

152. The inhibitor for use according to any one of claims 48, 92, 136, or 139 to 149, or the use according to any one of claims 49, 50, 93, 94, 137, 138, 148 or 149, wherein the FGFR3 genetic alteration is a gene fusion.

153. The inhibitor for use or the use according to claim 152, wherein FGFR3 gene fusion is FGFR3-TACC3, FGFR3-BAIAP2L1, or any combination thereof.

154. The inhibitor for use according to any one of claims 48, 92, 136, or 139 to 149, or the use according to any one of claims 49, 50, 93, 94, 137, 138, 148 or 149, wherein the FGFR3 genetic alteration is a gene mutation.

155. The inhibitor for use or the use according to claim 154, wherein the FGFR3 gene mutation is R248C, S249C, G370C, Y373C, or any combination thereof.

156. The inhibitor for use according to any one of claims 48, 92, 136, or 139 to 155 or the use according to any one of claims 49, 50, 93, 94, 137, 138, 148 to 155, wherein the FGFR inhibitor is erdafitinib.

157. The inhibitor for use or the use according to claim 156, wherein erdafitinib is administered daily.

158. The inhibitor for use or the use according to claim 156 or 157, wherein erdafitinib is administered orally.

159. The inhibitor for use or the use according to any one of claims 156 to 158, wherein erdafitinib is administered orally on a continuous daily dosing schedule.

160. The inhibitor for use or the use according to any one of claims 156 to 159, wherein erdafitinib is administered orally at a dose of about 8 mg once daily.


SEQUENCE LISTING (49 sequences)

SEQ ID NO: 1 - DNA, 268 bp, Artificial Sequence
  source: /note=“Description of Artificial Sequence Synthetic polynucleotide”
tcggaccgcggcaactacacctgcgtcgtggagaacaagtttggcagcatccggcagacgtacacgctggacgtgctggagtgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctca

SEQ ID NO: 2 - DNA, 378 bp, Artificial Sequence
  source: /note=“Description of Artificial Sequence Synthetic polynucleotide”
gaccgcggcaactacacctgcgtcgtggagaacaagtttggcagcatccggcagacgtacacgctggacgtgctgggtgagggccctggggcggcgcgggggtgggggcggcagtggcggtggtggtgagggagggggtggcccctgagcgtcatctgcccccacagagcgctgcccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaaggtgggccaccgtgtgcacgt

SEQ ID NO: 3 - DNA, 234 bp, Artificial Sequence
  source: /note=“Description of Artificial Sequence Synthetic polynucleotide”
gcgggcaattctattgggttttctcatcactctgcgtggctggtggtgctgccagccgaggaggagctggtggaggctgacgaggcgtgcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccg

SEQ ID NO: 4 - DNA, 301 bp, Artificial Sequence
  source: /note=“Description of Artificial Sequence Synthetic polynucleotide”
ctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtgtgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagc

SEQ ID NO: 5 - DNA, 21 bp, Artificial Sequence
  source: /note=“Description of Artificial Sequence Synthetic primer”
gacctggaccgtgtccttacc

SEQ ID NO: 6 - DNA, 21 bp, Artificial Sequence
  source: /note=“Description of Artificial Sequence Synthetic primer”
cttccccagttccaggttctt

SEQ ID NO: 7 - DNA, 20 bp, Artificial Sequence
  source: /note=“Description of Artificial Sequence Synthetic primer”
aggacctggaccgtgtcctt

SEQ ID NO: 8 - DNA, 20 bp, Artificial Sequence
  source: /note=“Description of Artificial Sequence Synthetic primer”
tataggtccggtggacaggg

SEQ ID NO: 9 - DNA, 20 bp, Artificial Sequence
  source: /note=“Description of Artificial Sequence Synthetic primer”
ctggaccgtgtccttaccgt

SEQ ID NO: 10 - DNA, 20 bp, Artificial Sequence
  source: /note=“Description of Artificial Sequence Synthetic primer”
gcagcccaggattgaactgt

SEQ ID NO: 11 - DNA, 23 bp, Artificial Sequence
  source: /note=“Description of Artificial Sequence Synthetic primer”
tggatcgaattctcactctcaca

SEQ ID NO: 12 - DNA, 21 bp, Artificial Sequence
  source: /note=“Description of Artificial Sequence Synthetic primer”
gccaagcaatctgcgtatttg

SEQ ID NO: 13 - DNA, 23 bp, Artificial Sequence
  source: /note=“Description of Artificial Sequence Synthetic primer”
gctcttcaatacagccctgatca

SEQ ID NO: 14 - DNA, 24 bp, Artificial Sequence
  source: /note=“Description of Artificial Sequence Synthetic primer”
acttggatcgaattctcactctca

SEQ ID NO: 15 - DNA, 23 bp, Artificial Sequence
  source: /note=“Description of Artificial Sequence Synthetic primer”
tggatcgaattctcactctcaca

SEQ ID NO: 16 - DNA, 25 bp, Artificial Sequence
  source: /note=“Description of Artificial Sequence Synthetic primer”
gcaaagcctgaattttcttgaataa

SEQ ID NO: 17 - DNA, 18 bp, Artificial Sequence
  source: /note=“Description of Artificial Sequence Synthetic primer”
gcatccggcagacgtaca

SEQ ID NO: 18 - DNA, 15 bp, Artificial Sequence
  source: /note=“Description of Artificial Sequence Synthetic primer”
ccccgcctgcaggat

SEQ ID NO: 19 - DNA, 18 bp, Artificial Sequence
  source: /note=“Description of Artificial Sequence Synthetic primer”
gcatccggcagacgtaca

SEQ ID NO: 20 - DNA, 15 bp, Artificial Sequence
  source: /note=“Description of Artificial Sequence Synthetic primer”
ccccgcctgcaggat

SEQ ID NO: 21 - DNA, 19 bp, Artificial Sequence
  source: /note=“Description of Artificial Sequence Synthetic primer”
aggagctggtggaggctga

SEQ ID NO: 22 - DNA, 19 bp, Artificial Sequence
  source: /note=“Description of Artificial Sequence Synthetic primer”
ccgtagctgaggatgcctg

SEQ ID NO: 23 - DNA, 18 bp, Artificial Sequence
  source: /note=“Description of Artificial Sequence Synthetic primer”
ctggtggaggctgacgag

SEQ ID NO: 24 - DNA, 16 bp, Artificial Sequence
  source: /note=“Description of Artificial Sequence Synthetic primer”
agcccaccccgtagct

SEQ ID NO: 25 - DNA, 21 bp, Artificial Sequence
  source: /note=“Description of Artificial Sequence Synthetic primer”
gtcgtggagaacaagtttggc

SEQ ID NO: 26 - DNA, 17 bp, Artificial Sequence
  source: /note=“Description of Artificial Sequence Synthetic primer”
gtctggttggccggcag

SEQ ID NO: 27 - DNA, 21 bp, Artificial Sequence
  source: /note=“Description of Artificial Sequence Synthetic primer”
gtcgtggagaacaagtttggc

SEQ ID NO: 28 - DNA, 17 bp, Artificial Sequence
  source: /note=“Description of Artificial Sequence Synthetic primer”
gtctggttggccggcag

SEQ ID NO: 29 - DNA, 19 bp, Artificial Sequence
  source: /note=“Description of Artificial Sequence Synthetic primer”
aggagctggtggaggctga

SEQ ID NO: 30 - DNA, 19 bp, Artificial Sequence
  source: /note=“Description of Artificial Sequence Synthetic primer”
ccgtagctgaggatgcctg

SEQ ID NO: 31 - DNA, 16 bp, Artificial Sequence
  source: /note=“Description of Artificial Sequence Synthetic primer”
gacgaggcgggcagtg

SEQ ID NO: 32 - DNA, 19 bp, Artificial Sequence
  source: /note=“Description of Artificial Sequence Synthetic primer”
gaagaagcccaccccgtag

SEQ ID NO: 33 - DNA, 2850 bp, Artificial Sequence
  source: /note=“Description of Artificial Sequence Synthetic polynucleotide”
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 34 - DNA, 2955 bp, Artificial Sequence
  source: /note=“Description of Artificial Sequence Synthetic polynucleotide”
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgtgccaggcccacccccaggtgttcccgcgcctgggggcccacccctgtccaccggacctatagtggacctgctccagtacagccagaaggacctggatgcagtggtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 35 - DNA, 3765 bp, Artificial Sequence
  source: /note=“Description of Artificial Sequence Synthetic polynucleotide”
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagtcctggatcagtgagagtgtggaggccgacgtgcgcctccgcctggccaatgtgtcggagcgggacgggggcgagtacctctgtcgagccaccaatttcataggcgtggccgagaaggccttttggctgagcgttcacgggccccgagcagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacaatgttatggaacagttcaatcctgggctgcgaaatttaataaacctggggaaaaattatgagaaagctgtaaacgctatgatcctggcaggaaaagcctactacgatggagtggccaagatcggtgagattgccactgggtcccccgtgtcaactgaactgggacatgtcctcatagagatttcaagtacccacaagaaactcaacgagagtcttgatgaaaattttaaaaaattccacaaagagattatccatgagctggagaagaagatagaacttgacgtgaaatatatgaacgcaactctaaaaagataccaaacagaacacaagaataaattagagtctttggagaaatcccaagctgagttgaagaagatcagaaggaaaagccaaggaagccgaaacgcactcaaatatgaacacaaagaaattgagtatgtggagaccgttacttctcgtcagagtgaaatccagaaattcattgcagatggttgcaaagaggctctgcttgaagagaagaggcgcttctgctttctggttgataagcactgtggctttgcaaaccacatacattattatcacttacagtctgcagaactactgaattccaagctgcctcggtggcaggagacctgtgttgatgccatcaaagtgccagagaaaatcatgaatatgatcgaagaaataaagaccccagcctctacccccgtgtctggaactcctcaggcttcacccatgatcgagagaagcaatgtggttaggaaagattacgacaccctttctaaatgctcaccaaagatgccccccgctccttcaggcagagcatataccagtcccttgatcgatatgtttaataacccagccacggctgccccgaattcacaaagggtaaataattcaacaggtacttccgaagatcccagtttacagcgatcagtttcggttgcaacgggactgaacatgatgaagaagcagaaagtgaagaccatcttcccgcacactgcgggctccaacaagaccttactcagctttgcacagggagatgtcatcacgctgctcatccccgaggagaaggatggctggctctatggagaacacgacgtgtccaaggcgaggggttggttcccgtcgtcgtacacgaagttgctggaagaaaatgagacagaagcagtgaccgtgcccacgccaagccccacaccagtgagaagcatcagcaccgtgaacttgtctgagaatagcagtgttgtcatccccccacccgactacttggaatgcttgtccatgggggcagctgccgacaggagagcagattcggccaggacgacatccacctttaaggccccagcgtccaagcccgagaccgcggctcctaacgatgccaacgggactgcaaagccgccttttctcagcggagaaaacccctttgccactgtgaaactccgcccgactgtgacgaatgatcgctcggcacccatcattcgatga

SEQ ID NO: 36 - DNA, 4989 bp, Artificial Sequence
  source: /note=“Description of Artificial Sequence Synthetic polynucleotide”
atggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgagatcatggaggaaacaaatacgcagattgcttggccatcaaaactgaagatcggagccaaatccaagaaagatccccatattaaggtttctggaaagaaagaagatgttaaagaagccaaggaaatgatcatgtctgtcttagacacaaaaagcaatcgagtcacactgaagatggatgtttcacatacagaacattcacatgtaatcggcaaaggtggcaacaatattaaaaaagtgatggaagaaaccggatgccatatccactttccagattccaacaggaataaccaagcagaaaaaagcaaccaggtatctatagcgggacaaccagcaggagtagaatctgcccgagttagaattcgggagctgcttcctttggtgctgatgtttgagctaccaattgctggaattcttcaaccggttcctgatcctaattccccctctattcagcatatatcacaaacgtacaatatttcagtatcatttaaacagcgttcccgaatgtatggtgctactgtcatagtacgagggtctcagaataacactagtgctgtgaaggaaggaactgccatgctgttagaacatcttgctgggagcttagcatcagctattcctgtgagcacacaactagatattgcagctcaacatcatctctttatgatgggtcgaaatgggagcaacatcaaacatatcatgcagagaacaggtgctcagatccactttcctgatcccagtaatccacaaaagaaatctaccgtctacctccagggcaccattgagtctgtctgtcttgcaaggcaatatctcatgggttgtcttcctcttgtgttgatgtttgatatgaaggaagaaattgaagtagatccacaattcattgcgcagttgatggaacagcttgatgtcttcatcagtattaaaccaaagcccaaacagccaagcaagtctgtgattgtgaaaagtgttgagcgaaatgccttaaatatgtatgaagcaaggaaatgtctcctcggacttgaaagcagtggggttaccatagcaaccagtccatccccagcatcctgccctgccggcctggcatgtcccagcctggatatcttagcttcagcaggccttggactcactggactaggtcttttgggacccaccaccttatctctgaacacttcaacaaccccaaactcactcttgaatgctcttaatagctcagtcagtcctttgcaaagtccaagttctggtacacccagccccacattatgggcacccccacttgctaatacttcaagtgccacaggtttttctgctataccacaccttatgattccatctactgcccaagccacattaactaatattttgttgtctggagtgcccacctatgggcacacagctccatctccccctcctggcttgactcctgttgatgtccatatcaacagtatgcagaccgaaggcaaaaaaatctctgctgctttaaatggacatgcacagtctccagatataaaatatggtgcaatatccacttcatcacttggagaaaaagtgctgagtgcaaatcacggggatccgtccatccagacaagtgggtctgagcagacatctcccaaatcaagccccactgaaggttgtaatgatgcttttgttgaagtaggcatgcctcgaagtccttcccattctgggaatgctggtgacttgaaacagatgatgtgtccctccaaggtttcctgtgccaaaaggcagacagtggaactattgcaaggcacgaaaaactcacacttacacagcactgacaggttgctctcagaccctgaactgagtgctaccgaaagccctttggctgacaagaaggctccagggagtgagcgcgctgcagagagggcagcagctgcccagcaaaactccgaaagggcccaccttgctccacggtcatcatatgtcaacatgcaggcatttgactatgaacagaagaagctattagccaccaaagctatgttaaagaaaccagtggtgacggaggtcagaacgcccacaaatacctggagtggcctgggtttttctaaatccatgccagctgaaactatcaaggagttgagaagggccaatcatgtgtcctataagcccacaatgacaaccacttatgagggctcatccatgtccctttcacggtccaacagtcgtgagcacttgggaggtggaagcgaatctgataactggagagaccgaaatggaattggacctggaagtcatagtgaatttgcagcttctattggcagccctaagcgtaaacaaaacaaatcaacggaacactatctcagcagtagcaattacatggactgcatttcctcgctgacaggaagcaatggctgtaacttaaatagctctttcaaaggttctgacctccctgagctcttcagcaaactgggcctgggcaaatacacagatgttttccagcaacaagagatcgatcttcagacattcctcactctcacagatcaggatctgaaggagctgggaataactacttttggtgccaggaggaaaatgctgcttgcaatttcagaactaaataaaaaccgaagaaagctttttgaatcgccaaatgcacgcacctctttcctggaaggtggagcgagtggaaggctaccccgtcagtatcactcagacattgctagtgtcagtggccgctggtag

SEQ ID NO: 37 - DNA, 3213 bp, Artificial Sequence
  source: /note=“Description of Artificial Sequence Synthetic polynucleotide”
atggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgagatggcagatgatcagggctgtattgaagagcagggggttgaggattcagcaaatgaagattcagtggatgctaagccagaccggtcctcgtttgtaccgtccctcttcagtaagaagaagaaaaatgtcaccatgcgatccatcaagaccacccgggaccgagtgcctacatatcagtacaacatgaattttgaaaagctgggcaaatgcatcataataaacaacaagaactttgataaagtgacaggtatgggcgttcgaaacggaacagacaaagatgccgaggcgctcttcaagtgcttccgaagcctgggttttgacgtgattgtctataatgactgctcttgtgccaagatgcaagatctgcttaaaaaagcttctgaagaggaccatacaaatgccgcctgcttcgcctgcatcctcttaagccatggagaagaaaatgtaatttatgggaaagatggtgtcacaccaataaaggatttgacagcccactttaggggggatagatgcaaaacccttttagagaaacccaaactcttcttcattcaggcttgccgagggaccgagcttgatgatggcatccaggccgactcggggcccatcaatgacacagatgctaatcctcgatacaagatcccagtggaagctgacttcctcttcgcctattccacggttccaggctattactcgtggaggagcccaggaagaggctcctggtttgtgcaagccctctgctccatcctggaggagcacggaaaagacctggaaatcatgcagatcctcaccagggtgaatgacagagttgccaggcactttgagtctcagtctgatgacccacacttccatgagaagaagcagatcccctgtgtggtctccatgctcaccaaggaactctacttcagtcaatag

SEQ ID NO: 38 - protein, 1210 aa, Homo sapiens [sequence omitted]

SEQ ID NO: 39 - protein, 53 aa, Homo sapiens
NSDSECPLSHDGYCLHDGVCMYIEALDKYACNCVVGYIGERCQYRDLKWWELR

SEQ ID NO: 40 - protein, 1408 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 41 - protein, 455 aa, Artificial Sequence
  source: /note=“Description of Artificial Sequence Synthetic polypeptide”
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 42 - protein, 214 aa, Artificial Sequence
  source: /note=“Description of Artificial Sequence Synthetic polypeptide”
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 449 aa, Artificial Sequence
  source: /note=“Description of Artificial Sequence Synthetic polypeptide”
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 44 - protein, 214 aa, Artificial Sequence
  source: /note=“Description of Artificial Sequence Synthetic polypeptide”
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 10 aa, Homo sapiens
CTGPGLEGCP

SEQ ID NO: 46 - protein, 10 aa, Homo sapiens
GEAPNQALLR

SEQ ID NO: 47 - protein, 10 aa, Homo sapiens
PGNESLKAML

SEQ ID NO: 48 - protein, 10 aa, Homo sapiens
SVIITASSCH

SEQ ID NO: 49 - DNA, 24 bp, Artificial Sequence
  source: /note=“Description of Artificial Sequence Synthetic oligonucleotide”
tttttttttttttttttttttttt